(19) United States
(12) Patent Application Publication
(10) Pub. No.: US 2022/0290163 A1
(43) Pub. Date: Sep. 15, 2022

(54) METHOD FOR MANIPULATING TERMINALS OF DOUBLE STRANDED DNA

(71) Applicant: BGI GENELAND SCIENTIFIC CO., LTD., Huankeyuan, Yixing, Jiangsu (CN)

(72) Inventors: Jiwei LIN, Huankeyuan, Yixing, Jiangsu (CN); Hongqi WANG, Huankeyuan, Yixing, Jiangsu (CN)

(73) Assignee: BGI GENELAND SCIENTIFIC CO., LTD., Huankeyuan, Yixing, Jiangsu (CN)

(21) Appl. No.: 17/629,498

(22) PCT Filed: Jul. 13, 2020
(86) PCT No.: PCT/CN2020/101669

§ 371 Date: Jan. 24, 2022

(30) Foreign Application Priority Data
Jul. 25, 2019 (CN) 201910676943.9

(51) Int. Cl. C12N 15/66

(52) U.S. Cl. CPC C12N 15/66

(57) ABSTRACT

A method for manipulating the terminals of a double-stranded DNA. The principle thereof is using a restriction nicking enzyme to first generate one or more nicks on one strand of a double-stranded DNA, then using an oligonucleotide adaptor to bind the same or a different restriction nicking enzyme to generate cleavage on the other strand of the double-stranded DNA, the position of cleavage being determined by the design of the oligonucleotide adapter, and eventually cleaving the double-stranded DNA of interest and generating various lengths of 5′ io protruding terminals and various lengths of 3′ protruding terminals or blunt terminals at the nicks. The bases at the terminals of a double-stranded DNA generated by means of this method can be designed at will, and such terminals can be used for double-stranded DNA splicing, particularly seamless splicing of double-stranded DNA.

### CROSS-REFERENCE TO RELATED APPLICATIONS

This application is a national phase entry under 35 U.S.C. § 371 of International Application PCT/CN2020/101669, filed Jul. 13, 2020, which claims priority to Chinese Patent Application No. 201910676943.9, filed Jul. 25, 2019, all of which are herein incorporated by reference in their entirety.

### STATEMENT REGARDING THE SEQUENCE LISTING

The Sequence Listing associated with this application is provided electronically in text format and is hereby incorporated by reference into the specification. The Sequence Listing is provided as a file entitled US17-629498_Sequence_Listing.txt created Mar. 18, 2022 which is about 34 kB in size.

### FIELD OF THE INVENTION

The present invention relates to a method of nucleic acid modification, in particular, a method for manipulating ends of double-stranded DNA.

### BACKGROUND OF THE INVENTION

Cleaving DNA from different sources and splicing them together as required is one of the most fundamental operations in molecular biology. There are two ways to splice two DNA fragments together, one is to use ligase to join the end-matched double-stranded DNA, i.e., ligase method, the DNA ends spliced in this way generally have an overhang of less than 4 bases; the other is to use homologous sequence between two DNA fragments for splicing, mainly the polymerase circular assembly (PCA), Gibson splicing method, etc, and the length of the homologous sequences is generally between a dozen bases to several thousand bases. The ligase method is simple and well established, and is still the main method of DNA splicing, where the spliced DNA is usually generated by restriction endonuclease hydrolysis.

Restriction endonucleases are a class of enzymes that perform double-stranded cleavage at specific positions on the DNA double-strand, and the results of the enzymatic cleavage are usually precise and predictable. As the first generation of gene editing tools at human disposal, restriction endonucleases still play an irreplaceable role. Over the years, new restriction endonucleases have been continuously discovered and new engineered restriction endonucleases have been screened. While scientific researchers have a choice among numerous restriction endonucleases, their toolbox is filled with a variety of restriction endonucleases that are either used frequently, occasionally, or not used at all. And it costs a lot of money to do so, because a commonly used restriction endonuclease costs between a few hundred and a few thousand CNY, and a toolbox that is said to be handy typically requires a dozen to hundreds of restriction endonucleases. The storage time of restriction endonucleases is typically between a few months and two years, for which they need to be renewed periodically.

In this ongoing quest to enrich the toolbox, there have also been attempts to obtain a universal restriction endonuclease. U.S. Pat. No. 4,935,357A mentions an enzyme digestion that can cleave at any position in single-stranded DNA, and the restriction endonucleases used are of type IIS, a class of restriction endonucleases whose recognition sequence and cleavage position do not overlap, such as FokI, whose recognition sequence is GGATG, but the cleavage position is between the subsequent ninth and tenth bases of the strand in which GGATG is located and between the subsequent thirteenth and fourteenth bases on the other strand (FIG. 1), yielding a 5′ overhanging end of 4 bases. In actual enzyme digestion, the recognition sequence of FokI does not need to be located on the same contiguous double strand as the enzyme digestion position, such as the sense strand in FIG. 1. If the base following GGATG comes from another single strand, which is able to hybridize with the base of the antisense strand, FokI can also cleave this single strand, and this single strand does not need to have the FokI recognition sequence. At this point the antisense strand plus the first half of the sense strand constitutes an adapter, and this adapter can be used for any single strand cleavage as long as the base composition on the antisense strand is adjusted. The limitation of this method is that the substrate it acts on is a single strand, and the product after cleavage is also a single strand, and cannot be used directly for splicing by ligase method.

Restriction nicking enzymes are a class of DNA endonucleases similar to restriction endonucleases, but unlike restriction endonucleases, they cleave only one of the strands of the double-stranded DNA in which their recognition sequence is located and do not generate a cleavage in the other strand. Therefore, DNA generated by restriction nicking enzyme hydrolysis will not have the effect of double-strand breaks if the two recognition sequences are not in close proximity and located on different strands.

### SUMMARY OF THE INVENTION

Therefore, the present invention relates to the following technical solutions.

A first aspect of the present invention provides a method of generating a predetermined end of a double-stranded DNA, which comprises:

generating one or more nicks at a predetermined position on one strand of a target double-stranded DNA using a restriction nicking enzyme to generate a single-stranded region on the other strand of the target double-stranded DNA;

using an oligonucleotide adapter having a recognition site for said restriction nicking enzyme to hybridize with said single-stranded region in combination with use of the same restriction nicking enzyme to generate a cleavage at a predetermined position on the other strand of the target double-stranded DNA, eventually cleaving the target double-stranded DNA and generating a predetermined end at the cleavage site;

wherein the predetermined end is a 3′ overhang, a blunt end or a 5′ overhang;

wherein the recognition sequence of the restriction nicking enzyme does not overlap with the cleavage position;

wherein said oligonucleotide adapter is a DNA molecule having a double-stranded portion and a single-stranded portion, wherein said oligonucleotide adapter contains in the double-stranded portion the recognition site for said restriction nicking enzyme but lacks a sequence cleavable by said restriction nicking enzyme, wherein the single-stranded portion of said oligonucleotide adapter is capable of hybridizing with the single-stranded region of the target double-stranded DNA to form a double-stranded structure that is recognizable by said restriction nicking enzyme, and thereby leaves the predetermined position on the other strand of the target double-stranded DNA in a position that can be cleaved by said restriction nicking enzyme by recognition of the recognition site in the oligonucleotide adapter by said restriction nicking enzyme.

In some embodiments, said restriction nicking enzyme is Nt.AlwI, Nt.BsmAI, Nt.BspQI, Nb.BsrDI, Nt.BstNBI or Nb.BtsI.

In some embodiments, said target double-stranded DNA is obtained by adding to the double-stranded DNA to be modified a double-stranded DNA fragment containing the restriction nicking enzyme recognition sequence, said double-stranded DNA fragment is added at a position such that said restriction nicking enzyme is capable of generating one or more nicks at said predetermined position by recognizing the added recognition sequence.

In some embodiments, said target double-stranded DNA is linear and contains the recognition sequence for said restriction nicking enzyme at a position near one end thereof, wherein the predetermined position on one strand of said target double-stranded DNA overlaps with a cleavage site determined according to the recognition sequence, and a cleavage is generated at that predetermined position using the restriction nicking enzyme, causing a single strand of DNA containing the recognition sequence between the cleavage site and the end of this side to dissociate from the double strand, thereby generating a single-stranded region on the other strand of the target double-stranded DNA.

In some embodiments, said target double-stranded DNA is obtained by adding to one end of the linear double-stranded DNA to be modified a double-stranded DNA fragment containing the restriction nicking enzyme recognition sequence, said double-stranded DNA fragment is added at a position such that said predetermined position on the target double-stranded DNA overlaps with a cleavage site determined according to the recognition sequence, the single strand of DNA dissociated from the double strand after generating a cleavage at the predetermined position using the restriction nicking enzyme is the single strand of DNA containing the recognition sequence and located between the cleavage site and the free end of the added double-stranded DNA fragment.

In some embodiments, said target double-stranded DNA contains at least two recognition sequences of same sequence on the same strand, said at least two recognition sequences are oriented in the same direction and in close proximity to each other, the predetermined position on one strand of said target double-stranded DNA overlaps with a cleavage site determined according to one of the recognition sequences, each of said at least two recognition sequences is cleaved once at the cleavage site using said restriction nicking enzyme, causing the single strand of DNA comprising the recognition sequence and located between said at least two cleavage sites to dissociate from the double strand, thereby generating a single-stranded region on the other strand of the target double-stranded DNA.

In some embodiments, said target double-strand is obtained by adding to the double-stranded DNA to be modified a double-stranded DNA fragment containing said at least two recognition sequences, said double-stranded DNA fragment is added at a position such that said predetermined position on the target double-stranded DNA overlaps with a cleavage site determined according to one of the recognition sequences.

In some embodiments, said target double-stranded DNA contains on each of the two strands a double-stranded DNA fragment containing at least two recognition sequences of same sequence, said at least two recognition sequences are in opposite directions and in close proximity to each other, said predetermined position on the target double-stranded DNA overlapsd with a cleavage site determined according to one of the recognition sequences, each of said at least two recognition sequences is cleaved once at the cleavage site using said restriction nicking enzyme, causing the double strand between the two cleavage sites to dissociate, thereby generating a single-stranded region on the other strand of the target double-stranded DNA.

In some embodiments, said target double-strand is obtained by adding to the double-stranded DNA to be modified a double-stranded DNA fragment containing said at least two recognition sequences, said double-stranded DNA fragment is added at a position such that said predetermined position on the target double-stranded DNA overlaps with a cleavage site determined according to one of the recognition sequences.

In some embodiments, in the double-stranded DNA fragment added to the double-stranded DNA to be modified, at least one restriction nicking enzyme recognition sequence is immediately adjacent to the end of its cleavage site side, such that after the addition of said double-stranded DNA fragment, said restriction nicking enzyme recognition sequence is immediately adjacent to the double-stranded DNA to be modified on its cleavage site side;

alternatively, the double-stranded DNA fragment added to the double-stranded DNA to be modified has one or more nucleotides between at least one restriction nicking enzyme recognition sequence and the end of its cleavage site side, such that after the addition of said double-stranded DNA fragment, said restriction nicking enzyme recognition sequence has one or more of said nucleotides between its cleavage site side and the double-stranded DNA to be modified.

In some embodiments, said dissociation is carried out at 30 to 75 degrees Celsius, preferably at 45 to 65 degrees Celsius, more preferably at 53 to 63 degrees Celsius.

In some embodiments, the resulting single-stranded region has a length of 5 to 50 bases, preferably 10 to 30 bases, more preferably 15 to 20 bases.

In some embodiments, the oligonucleotide adapter is formed by hybridization of two oligonucleotides, the double-stranded portion is the portion of the two oligonucleotides that hybridize, the single-stranded portion is the portion of the two oligonucleotides that do not participate in the hybridization, and the restriction nicking enzyme recognition site is located in the double-stranded portion; alternatively the oligonucleotide adapter consists of an oligonucleotide that can form a hairpin structure, the stem of the hairpin includes a hybridized double-stranded portion and a single-stranded portion, and the restriction nicking enzyme recognition site is located in the double-stranded portion of the stem.

In some embodiments, the restriction nicking enzyme recognition sequence in said oligonucleotide adapter is immediately adjacent to the end of the double-stranded portion on its cleavage site side, or wherein the restriction nicking enzyme recognition sequence in the oligonucleotide adapter is separated by one, two, or more nucleotides from the end of the double-stranded portion on its cleavage site side.

In some embodiments, the region where the other strand of said target double-stranded DNA hybridizes with the single-stranded portion of the oligonucleotide adapter is located on its single-stranded region and is immediately adjacent to its double-stranded region, or the region where the other strand of said target double-stranded DNA hybridizes with the single-stranded portion of the oligonucleotide adapter is located on its single-stranded region and is separated by one, two or more nucleotides from its double-stranded region.

In some embodiments, the restriction nicking enzyme used is Nt.AlwI or Nt.BstNBI, the number of nucleotides between the recognition sequence and 3′ end of the strand in which it is located in said oligonucleotide adapter is 2, the number of nucleotides separating the hybridization region of the single-stranded region from the double-stranded region of the target double-stranded DNA is 2, eventually resulting in a 3′ overhang of 4 bases.

In some embodiments, the oligonucleotide adapter used is a mixture of 16 oligonucleotide adapters, the two single-stranded nucleotides immediately adjacent to the double-stranded portion of said 16 oligonucleotide adapters are different and all other nucleotides are identical, and said two single-stranded nucleotides immediately adjacent to the double-stranded portion include all permutations of the four kinds of nucleotides at these two positions.

In some embodiments, the region on said target double-stranded DNA that hybridizes with the single-stranded portion of the oligonucleotide adapter comprises the single-stranded region of that target double-stranded DNA and a portion of the double-stranded region sequence adjacent to that single-stranded region, said portion of the double-stranded region sequence adjacent to that single-stranded region is referred to as an invaded region; said single-stranded portion of the oligonucleotide adapter comprises a sequence capable of hybridizing with the single-stranded region of the target double-stranded DNA, and between that sequence and the double-stranded portion of the oligonucleotide adapter also includes a sequence capable of hybridizing with a segment of the double-stranded region of the target double-stranded DNA adjacent to the single-stranded region, which is referred to as an invading region.

In some embodiments, the length of the invading region is between 1 to 100 bases, preferably between 1 to 30 bases, more preferably between 3 to 20 bases.

In some embodiments, the number of characteristic nucleotides of the restriction nicking enzyme used minus the number of nucleotides spaced between the recognition sequence in the oligonucleotide adapter used and the end of its cleavage site side is greater than the number of nucleotides in the invading region, wherein the cleavage site of the restriction nicking enzyme used is on 3′ side of its recognition sequence, eventually generating a 3′ overhang, the length of the overhang is equal to the number of characteristic nucleotides of the restriction nicking enzyme used minus the number of nucleotides spaced between the recognition sequence in the oligonucleotide adapter used and the end of its cleavage site side, then minus the number of nucleotides in the invading region;

alternatively, wherein the number of characteristic nucleotides of the restriction nicking enzyme used minus the number of nucleotides spaced between the recognition sequence in the oligonucleotide adapter used and the end of its cleavage site side is greater than the number of nucleotides in the invading region, wherein the cleavage site of the restriction nicking enzyme used is on 5′ side of its recognition sequence, eventually generating a 5′ overhang, the length of the overhang is the number of characteristic nucleotides of the restriction nicking enzyme used minus the number of nucleotides spaced between the recognition sequence in the oligonucleotide adapter used and the end of its cleavage site side, then minus the number of nucleotides in the invading region.

In some embodiments, the number of characteristic nucleotides of the restriction nicking enzyme used minus the number of nucleotides spaced between the recognition sequence in the oligonucleotide adapter used and the end of its cleavage site side is equal to the number of nucleotides in the invading region, eventually resulting in a blunt end.

In some embodiments, the number of characteristic nucleotides of the restriction nicking enzyme used minus the number of nucleotides spaced between the recognition sequence in the oligonucleotide adapter used and the end of its cleavage site side is less than the number of nucleotides in the invading region, wherein the cleavage site of the restriction nicking enzyme used is on 3′ side of its recognition sequence, eventually generating a 5′ overhang, the length of the overhang is the difference obtained by the number of nucleotides in the invading region minus a number obtained by subtracting the number of nucleotides spaced between the recognition sequence in the oligonucleotide adapter used and the end of its cleavage site side from the number of characteristic nucleotides of the restriction nicking enzyme used;

alternatively, the number of characteristic nucleotides of the restriction nicking enzyme used minus the number of nucleotides spaced between the recognition sequence in the oligonucleotide adapter used and the end of its cleavage site side is less than the number of nucleotides in the invading region, wherein the cleavage site of the restriction nicking enzyme used is on 5′ side of its recognition sequence, eventually generating a 3′ overhang, the length of the overhang is the difference obtained by the number of nucleotides in the invading region minus a number obtained by subtracting the number of nucleotides spaced between the recognition sequence in the oligonucleotide adapter used and the end of its cleavage site side from the number of characteristic nucleotides of the restriction nicking enzyme used.

In some embodiments, said method is carried out at a fixed temperature being between 37 to 75 degrees Celsius, preferably between 45 to 65 degrees Celsius, more preferably 55 degrees Celsius.

In some embodiments, said method is carried out under a temperature cycle; the maximum temperature of the cycle is between 50 to 75 degrees Celsius, preferably 55 to 65 degrees Celsius; the minimum temperature of the cycle is between 37 to 55 degrees Celsius, preferably 45 to 55 degrees Celsius; and the duration of each cycle is 30 seconds to 20 minutes, preferably 1 minute to 5 minutes.

In some embodiments, said method is carried out in the presence of D-trehalose.

A third aspect of the present invention provides a method of generating a cleavage at any predetermined position on a target single-stranded DNA, which comprises:

hybridizing a predetermined region of a target single-stranded DNA with a single-stranded portion of an oligonucleotide adapter, said oligonucleotide adapter is a DNA molecule having a double-stranded portion and a single-stranded portion, wherein the oligonucleotide adapter contains in the double-stranded portion a recognition site for the restriction nicking enzyme but lacks a sequence cleavable by the restriction nicking enzyme, wherein the single-stranded portion of the oligonucleotide adapter is capable of hybridizing with a predetermined region of the target single-stranded DNA to form a double-stranded structure that is recognizable by the restriction nicking enzyme, and thereby leaves the predetermined position on the target single-stranded DNA in a position that can be cleaved by said restriction nicking enzyme by recognition of the recognition site on the oligonucleotide adapter by said restriction nicking enzyme;

generating a cleavage at the predetermined position on said target single strand using said restriction nicking enzyme;

wherein the recognition sequence for said restriction nicking enzyme does not overlap with the cleavage position.

In some embodiments, said restriction nicking enzyme is Nt.AlwI, Nt.BsmAI, Nt.BspQI, Nb.BsrDI, Nt.BstNBI or Nb.BtsI.

In some embodiments, the oligonucleotide adapter is formed by hybridization of two oligonucleotides, the double-stranded portion is the portion of the two oligonucleotides that hybridize, the single-stranded portion is the portion of the two oligonucleotides that do not participate in the hybridization, and the restriction nicking enzyme recognition site is located in the double-stranded portion; alternatively the oligonucleotide adapter consists of an oligonucleotide that can form a hairpin structure with a stem as the double-stranded portion and a open loop as the single-stranded portion, and the restriction nicking enzyme recognition site is located in the double-stranded portion of the hairpin.

In some embodiments, said restriction nicking enzyme recognition sequence in said oligonucleotide adapter is immediately adjacent to the end of the double-stranded portion on its cleavage site side, or wherein the restriction nicking enzyme recognition sequence in the oligonucleotide adapter is separated by one, two, or more nucleotides from the end of the double-stranded portion on its cleavage site side.

In some embodiments, said target single-stranded DNA is an oligonucleotide synthesized by a DNA synthesizer, single-stranded DNA obtained from a plasmid with an f1 replicon under a phagemid rescue operation, single-stranded DNA generated in a rolling loop replication or single-stranded DNA obtained from double-stranded DNA under denaturing conditions.

A second aspect of the present invention provides a method of generating a predetermined end of a double-stranded DNA, which comprises:

generating one or more nicks at a predetermined position on one strand of a target double-stranded DNA using a first restriction nicking enzyme to generate a single-stranded region on the other strand of the target double-stranded DNA

using an oligonucleotide adapter having a recognition site for a second restriction nicking enzyme to hybridize with said single-stranded region in combination with use of the second restriction nicking enzyme to generate a cleavage at a predetermined position on the other strand of the target double-stranded DNA, eventually cleaving the target double-stranded DNA and generating the predetermined end at the cleavage site;

wherein said oligonucleotide adapter is a DNA molecule having a double-stranded portion and a single-stranded portion, said oligonucleotide adapter comprises the recognition site for said second restriction nicking enzyme and further comprises a complementary sequence to the cleavage site of said second restriction nicking enzyme but lacks a sequence that can be cleaved by said second restriction nicking enzyme, the single-stranded portion of said oligonucleotide adapter hybridizes with the single-stranded region of the target double-stranded DNA and forms, together with the double-stranded portion of said oligonucleotide adapter, a double-stranded structure recognizable and cleavable by said second restriction nicking enzyme, and thereby leaves the predetermined position on the other strand of the target double-stranded DNA in a position that can be cleaved by said restriction nicking enzyme by recognition of the recognition site on the oligonucleotide adapter by said second restriction nicking enzyme;

said first restriction nicking enzyme is the same as or different from said second restriction nicking enzyme.

In some embodiments, the first restriction nicking enzyme is a restriction nicking enzyme whose recognition sequence does not overlap with the cleavage position, or whose recognition sequence overlaps with the cleavage position; the second restriction nicking enzyme is a restriction nicking enzyme whose recognition sequence does not overlap with the cleavage position.

In some embodiments, the second restriction nicking enzyme is Nt.AlwI, Nt.BsmAI, Nt.BspQI, Nb.BsrDI, Nt.BstNBI, or Nb.BtsI.

In some embodiments, the first restriction nicking enzyme is Nt.AlwI, Nt.BsmAI, Nt.BspQI, Nb.BsrDI, Nt.BstNBI, Nb.BtsI, Nt.BbvCI, Nb.BbvCI, Nb.BsmI or Nb.BssSI.

In some embodiments, said generating a single-stranded region is to generate one nick or two nicks at the predetermined position on one strand of the target double-stranded DNA using the first restriction nicking enzyme such that a single-stranded region is generated after denaturing separation of the DNA double-strand between one nick and an end of the target double-stranded DNA or the double-stranded DNA between the two nicks.

In some embodiments, said two nicks are located on the same strand of the target double-stranded DNA, or on different strands of the target double-stranded DNA.

In some embodiments, said dissociation is carried out at 30 to 75 degrees Celsius, preferably at 45 to 65 degrees Celsius, more preferably at 53 to 63 degrees Celsius.

In some embodiments, the resulting single-stranded region has a length of 1 to 100 bases, preferably 5 to 50 bases, more preferably 10 to 30 bases, and more preferably 15 to 20 bases.

In some embodiments, said oligonucleotide adapter comprises a double-stranded portion and a single-stranded portion, the oligonucleotide adapter comprises the second restriction nicking enzyme recognition sequence but lacks a sequence that can be cleaved by the second restriction nicking enzyme and has only its complementary sequence, the complementary sequence constitutes the single-stranded portion of said oligonucleotide adapter; the single-stranded portion of the oligonucleotide adapter can hybridize with the single-stranded region of the target double-stranded DNA, and the structure formed by hybridization of the oligonucleotide adapter and the target double-stranded DNA can be recognized by the second restriction nicking enzyme and cleaved at a predetermined position in or near the single-stranded region of the target double-stranded DNA.

In some embodiments, said oligonucleotide adapter is formed by hybridization of two oligonucleotide chains, the double-stranded portion is the portion of the two oligonucleotides that hybridize, the single-stranded portion is the portion of the two oligonucleotides that do not participate in the hybridization.

In some embodiments, said oligonucleotide adapter consists of an oligonucleotide chain that can form a hairpin structure, with the double-stranded portion being the stem portion of the hairpin and the single-stranded portion being the open-loop portion of the hairpin.

In some embodiments, the region of the other strand of said target double-stranded DNA that hybridizes with the single-stranded portion of the oligonucleotide adapter is immediately adjacent to the double-stranded portion of said oligonucleotide adapter, and the region of the other strand of said target double-stranded DNA that hybridizes with the single-stranded portion of the oligonucleotide adapter is located on the single-stranded region of said target double-stranded DNA and is immediately adjacent to or one, two or more nucleotides apart from the double-stranded region of said target double-stranded DNA.

In some embodiments, said second restriction nicking enzyme is Nt.BstNBI, Nt.AlwI, Nt.BspQI or Nt.BsmAI, said second restriction nicking enzyme recognition sequence in said oligonucleotide adapter is located in the double-stranded portion and said second restriction nicking enzyme recognition sequence is immediately adjacent to the end of the double-stranded portion on its cleavage site side, or the restriction nicking enzyme recognition sequence in the oligonucleotide adapter is separated by one, two, or more nucleotides from the end of the double-stranded portion on its cleavage site side.

In some embodiments, the second restriction nicking enzyme used is Nt.AlwI or Nt.BstNBI, the number of nucleotides between the recognition sequence and 3′ end of the strand in which it is located in said oligonucleotide adapter is 2, and the number of nucleotides separating the hybridization region of the single-stranded region from the double-stranded region of the target double-stranded DNA is 2, eventually resulting in a 3′ overhang of 4 bases; the oligonucleotide adapter used is a mixture of 16 oligonucleotide adapters, the two single-stranded nucleotides immediately adjacent to the double-stranded portion of said 16 oligonucleotide adapters are different and all other nucleotides are identical, and said two single-stranded nucleotides immediately adjacent to the double-stranded portion include all permutations of the four kinds of nucleotides at these two positions.

In some embodiments, said second restriction nicking enzyme is Nb.BsrDI or Nb.BtsI, the second restriction nicking enzyme recognition sequence in said oligonucleotide adapter is located on the strand having a single-stranded portion of the two strands of the double-stranded portion, one nucleotide at 3′ end of the recognition sequence is located on the single-stranded portion, other nucleotides of the recognition sequence are located on the double-stranded portion, and a partial sequence of the complementary sequence of the recognition sequence on the minus strand is located on the double-stranded portion and immediately adjacent to its 5′ end, said partial sequence is the nucleotides other than the last nucleotide at 5′ end of the complementary sequence on the minus strand.

In some embodiments, the region of the other strand of said target double-stranded DNA that hybridizes with the single-stranded portion of the oligonucleotide adapter is immediately adjacent to the double-stranded portion of said oligonucleotide adapter; and the region on said target double-stranded DNA that hybridizes to the single-stranded portion of the oligonucleotide adapter includes not only the single-stranded region of that target double-stranded DNA, but also a portion of the double-stranded region sequence adjacent to that single-stranded region, said portion of the double-stranded region sequence adjacent to that single-stranded region is referred to as an invaded region; the single-stranded portion of said oligonucleotide adapter comprises a sequence capable of hybridizing with the single-stranded region of the target double-stranded DNA, and between that sequence and the double-stranded portion of the oligonucleotide adapter also includes a sequence capable of hybridizing with a segment of the double-stranded region of the target double-stranded DNA adjacent to the single-stranded region, which is referred to as an invading region.

In some embodiments, the length of the invading region is between 1 to 100 bases, preferably between 1 to 30 bases, more preferably between 3 to 20 bases.

In some embodiments, said second restriction nicking enzyme is Nt.BstNBI, Nt.AlwI, Nt.BspQI or Nt.BsmAI, the second restriction nicking enzyme recognition sequence in said oligonucleotide adapter is located in the double-stranded portion and said second restriction nicking enzyme recognition sequence is immediately adjacent to the end of the double-stranded portion on its cleavage site side, or the restriction nicking enzyme recognition sequence in the oligonucleotide adapter is separated by one, two, or more nucleotides from the end of the double-stranded portion on its cleavage site side.

In some embodiments, said second restriction nicking enzyme is Nb.BsrDI or Nb.BtsI, the second restriction nicking enzyme recognition sequence in said oligonucleotide adapter is located on the strand having a single-stranded portion of the two strands of the double-stranded portion, one nucleotide at 3′ end of the recognition sequence is located on the single-stranded portion, other nucleotides of the recognition sequence are located on the double-stranded portion, and a partial sequence of the complementary sequence of the recognition sequence on the minus strand is located on the double-stranded portion and immediately adjacent to its 5′ end, said partial sequence is the nucleotides other than the last nucleotide at 5′ end of the complementary sequence on the minus strand.

In some embodiments, said method is carried out at a fixed temperature being between 37 to 75 degrees Celsius, preferably between 45 to 65 degrees Celsius, more preferably 55 degrees Celsius.

In some embodiments, said method is carried out under a temperature cycle; the maximum temperature of the cycle is between 50 to 75 degrees Celsius, preferably 55 to 65 degrees Celsius; the minimum temperature of the cycle is between 37 to 55 degrees Celsius, preferably 45 to 55 degrees Celsius; and the duration of each cycle is 30 seconds to 20 minutes, preferably 1 minute to 5 minutes.

In some embodiments, said method is carried out in the presence of D-trehalose.

In some embodiments, said first restriction nicking enzyme is different from said second restriction nicking enzyme and said target double-stranded DNA is methylated using a methylase of said second restriction nicking enzyme prior to generation of a single-stranded region of said double-stranded target DNA.

In some embodiments, said methylation is carried out in vitro, or in vivo in a host cell.

In some embodiments, said methylation is carried out in vivo in a host cell, wherein said target double-stranded DNA is located on the same DNA double-strand as the gene encoding said methylase, or wherein the gene encoding said methylase is located on a host cell chromosome such that the host cell expresses said methylase.

In some embodiments, said second restriction nicking enzyme is Nt.BstNBI or Nt.AlwI and said methylase is M.BstNBI and M.AlwI.

In some embodiments, said first restriction nicking enzyme is Nt.BspQI or Nb.BbvCI or Nt.BbvCI.

### DETAILED DESCRIPTION OF THE INVENTION

There is a class of restriction nicking enzymes whose recognition sequence and cleavage site do not overlap, such as Nt.AlwI, Nt.BstNBI, and the like, which is similar to FokI, the present invention found for the first time that these enzymes also allow the recognition sequence to be located on a different DNA molecule than the cleaved sequence. Use of such restriction nicking enzymes allows free manipulation of double-stranded DNA ends, and in most cases, only one restriction nicking enzyme is required to complete the recombination splicing operation of DNA, thus eliminating the need to purchase multiple restriction endonucleases, and because the length of the double-stranded DNA end overhangs can be customized, the assembly of large segments of DNA can be achieved in the long sticky-end mode. This method of manipulating the ends of double-stranded DNA is based on the principle of generating predetermined ends by a two-step cleavage, the first step of which involves the use of restriction nicking enzymes to first generate one or more nicks on one strand of double-stranded DNA to generate a single-stranded region on the other strand, and the second step of which involves the use of oligonucleotide adapters to hybridize with the single-stranded region in combination with the same restriction nicking enzyme to generate a cleavage on the other strand of the double-stranded DNA. The position of the cleavage correlates with the sequence selection of the oligonucleotide adapter, which ultimately cleaves the target double-stranded DNA and generates ends of customizable length and customizable types of overhangs at the cleavage.

In the present invention, unless otherwise specified, “the other strand” refers to the strand of double-stranded DNA that generates the single-stranded region and is cleaved in the second step.

A first aspect of the present invention provides a method of generating a predetermined end of a double-stranded DNA, which comprises:

generating one or more nicks at a predetermined position on one strand of a target double-stranded DNA using a restriction nicking enzyme to generate a single-stranded region on the other strand of the target double-stranded DNA;

using an oligonucleotide adapter having a recognition site for said restriction nicking enzyme to hybridize with said single-stranded region in combination with use of the same restriction nicking enzyme to generate a cleavage at a predetermined position on the other strand of the target double-stranded DNA, eventually cleaving the target double-stranded DNA and generating the predetermined end at the cleavage site;

wherein said oligonucleotide adapter is a DNA molecule having a double-stranded portion and a single-stranded portion, wherein said oligonucleotide adapter contains in the double-stranded portion the recognition site for said restriction nicking enzyme but lacks a sequence cleavable by said restriction nicking enzyme, wherein the single-stranded portion of said oligonucleotide adapter is capable of hybridizing with a single-stranded region of the target double-stranded DNA to form a double-stranded structure that is recognizable by said restriction nicking enzyme, and thereby leaves the predetermined position on the other strand of the target double-stranded DNA in a position that can be cleaved by said restriction nicking enzyme by recognition of the recognition site on the oligonucleotide adapter by said restriction nicking enzyme.

It will be understood by those skilled in the art that in the above method of generating a predetermined double-stranded DNA end, by “a double-stranded structure that is recognizable by said restriction nicking enzyme” is meant that when the single-stranded portion of said oligonucleotide adapter hybridizes with the single-stranded region of the target double-stranded DNA, the hybridized region and the double-stranded portion of said oligonucleotide adapter together form a double-stranded structure recognizable and cleavable by said restriction nicking enzyme, said restriction nicking enzyme recognizes the recognition site on the double-stranded portion of said oligonucleotide adapter and cleaves the other strand of the target double-stranded DNA at the predetermined position.

It will be understood by those skilled in the art that the single-stranded region of the target double-stranded DNA hybridizes with the single-stranded portion of the oligonucleotide adapter to form a hybridized region immediately adjacent to the double-stranded portion of the oligonucleotide adapter, whereby said restriction nicking enzyme recognizes the recognition site on said double-stranded portion of the oligonucleotide adapter and cleaves the other strand of the target double-stranded DNA at the predetermined position.

The predetermined position on the other strand of the target double-stranded DNA being cleaved can be located in a single-stranded region on the other strand of the target double-stranded DNA or in a double-stranded region on the other strand of the target double-stranded DNA, which can be achieved by the design of oligonucleotide adapter.

The “predetermined end” described herein includes 3′ overhangs of different lengths, blunt ends, or 5′ overhangs of different lengths. In the present invention, the term “overhang” refers to the presence of unpaired single-stranded nucleotides at the end of double-stranded DNA, and the length of the overhang is the number of such unpaired single-stranded nucleotides. 3′ overhang means that the end of the overhanging base away from the double strand is the 3′ end, which can also be referred to as the 3′ protruding sticky end. 5′ overhang means that the end of the overhanging base away from the double strand is the 5′ end, which can also be referred to as the 5′ protruding sticky end. The length of the 3′ overhang or 5′ overhang obtained by the method of the present invention can be tailored to be between 0 and 50 bases, preferably 0 to 20 bases, more preferably 2 to 10 bases. Overhanging bases can be used for hybridization or ligation with other double-stranded DNA ends or single-stranded DNA, or as probes.

In the present invention, “nicking” or “incising” refers to cleaving only one strand of double-stranded DNA, therefore, in the present invention, “nicking” can be used interchangeably with “cleaving” of one strand of double-stranded DNA, and “cleavage site” and “enzyme digestion site” can be used interchangeably. The term “one or more nicks” can mean one, two, three, four or more nicks. Restriction nicking enzymes, which can also be referred to as restriction incisioin enzymes, nicking endonucleases, or incision endonucleases, are DNA endonucleases that recognize a specific sequence and cleave only one strand of double-stranded DNA at a defined position near the recognized sequence. Preferred restriction nicking enzymes are restriction nicking enzymes in which the recognition sequence does not overlap with the cleavage position, such as restriction nicking enzymes in which the recognition sequence is immediately adjacent to the cleavage position, or in which the recognition sequence is separated from the cleavage position by 1, 2, 3, 4, 5 or more nucleotides, including but not limited to: Nt.AlwI, Nt.BsmAI, Nt.BspQI, Nb.BsrDI, Nt.BstNBI, Nb.BtsI, preferably Nt.BstNBI and Nt.AlwI, more preferably Nt.BstNBI. The recognition sequences of these restriction nicking enzymes and their cleavage sites are well known to those skilled in the art, for example Nt.BstNBI recognizes 5′-GAGTC-3′ and cleaves between the 4^{th} and 5^{th} base after GAGTC, Nt.AlwI recognizes 5′-GGATC-3′ and cleaves between the 4^{th} and 5^{th} bases after GGATC, Nt.BsmAI recognizes 5′-GTCTC-3′ and cleaves between the 1^{st} and 2^{nd} bases after GTCTC, Nt.BspQI recognizes 5′-GCTCTTC-3′ and cleaves between the 1^{st} and 2^{nd} bases after GCTCTTC, Nb.BsrDI recognizes 3′-CGTTAC-5′ and cleaves the 5′ end of 3′-CGTTAC-5′ at a position immediately adjacent to this recognition sequence, and Nb.BtsI recognizes 3′-CGTCAC-5′ and cleaves the 5′ end of 3′-CGTCAC-5′ at a position immediately adjacent to this recognition sequence. As known to those skilled in the art, with respect to the recognition sequences of Nb.BtsI and Nb.BsrDI, it can also be described that Nb.BtsI recognizes 5′-GCAGTG-3′ and cleaves the 5′ end of the complementary sequence of the recognition sequence 3′-CGTTAC-5′ on the minus strand at a position immediately adjacent to the complementary sequence. Nb.BsrDI recognizes 5′-GCAATG-5′ and cleaves the 5′ end of the complementary sequence 3′-CGTCAC-5′ of the recognition sequence on the minus strand at a position immediately adjacent to the complementary sequence. The restriction nicking enzyme used in the present invention, when its recognition sequence does not overlap with the cleavage position, need not be located on the same contiguous double strand as the enzyme digestion position; the enzyme digestion position may be located on a single strand of another DNA molecule, provided that the single strand of said other DNA molecule is hybridizable to the complementary strand of the enzyme digestion position. For the purpose of the present invention, “recognition sequence” or “recognition sequence of a restriction nicking enzyme” refers to a specific sequence that can be recognized by the restriction nicking enzyme and in the vicinity of which one strand of double-stranded DNA is cleaved at a defined position. In some cases, when referring to a recognition sequence on a particular strand, the recognition sequence may refer to the sequence that is on the same strand as the cleavage site and recognizable by the restriction nicking enzyme. The number of nucleotides between the recognition sequence and the cleavage position is referred to in the present invention as the number of characteristic nucleotides of the restriction nicking enzyme.

In the present invention, target double-stranded DNA may refer to double-stranded DNA containing the recognition site for the restriction nicking enzyme and which can be cleaved by said restriction nicking enzyme to generate one or more nicks at the predetermined position on one of its strands, or to double-stranded DNA with a single-stranded region resulting from such target double-stranded DNA being cleaved by the restriction nicking enzyme. When some double-stranded DNA needs to be modified to have predetermined ends, these double-stranded DNAs do not necessarily have recognition sites for restriction nicking enzymes and may not be directly usable as said target double-stranded DNA. Thus, in some embodiments, a double-stranded DNA fragment containing the restriction nicking enzyme recognition sequence is added to the double-stranded DNA to be modified to obtain the target double-stranded DNA, the cleavage site determined by the recognition sequence is located on the double-stranded DNA to be modified, said double-stranded DNA fragment is added at a position such that said restriction nicking enzyme is capable of generating one or more nicks at the predetermined position on one strand of the target DNA double-strand by recognizing the added recognition sequence and thereby generating a single-stranded region on the other strand.

In the present invention, the double-stranded DNA to be modified is the double-stranded DNA that needs to be modified to obtain a predetermined end. The double-stranded DNA to be modified can be double-stranded DNA that needs to be spliced, and since performing splicing, especially seamless splicing, requires double-stranded DNA with specific ends, specific ends need to be generated on these double-stranded DNA. The double-stranded DNA to be modified can be linear or circular double-stranded DNA obtained by any method, such as linear double-stranded DNA obtained by PCR, or vector double-stranded DNA, such as a plasmid.

If the double-stranded DNA sequence to be modified happens to have the recognition site for said restriction nicking enzyme at a suitable position, the double-stranded DNA to be modified can be used directly as the target double-stranded DNA. However, since the probability of occurrence of restriction nicking enzyme recognition sites on random double-stranded DNA is small, in most cases the target double-stranded DNA is obtained by adding a double-stranded DNA fragment containing the restriction nicking enzyme recognition sequence to the double-stranded DNA to be modified. By adding a double-stranded DNA fragment containing the restriction nicking enzyme recognition sequence, it is possible to generate nicks at any predetermined position on the double-stranded DNA to be modified, without the restriction of the original recognition sequence in the double-stranded DNA to be modified.

In some embodiments, a single-stranded region can be generated by a single cleavage, i.e., by using the restriction nicking enzyme to generate a nick at a predetermined position on one strand of the target double-stranded DNA that is close to the end of one side of the target double-stranded DNA, causing a single-stranded segment of DNA between the nick and the end of that side to dissociate from the double strand, thereby generating a single-stranded region on the other strand. The dissociation occurs at the appropriate temperature at which the DNA double-strand between the nick and the end undergoes denaturation and separation. The approach is known as the single cleavage method in the present invention and can be applied when the double-stranded DNA to be modified is linear, such as PCR products, and the like. In some particular embodiments, the target double-stranded DNA contains the recognition sequence for said restriction nicking enzyme at a position proximate to the end of one side thereof, said predetermined position on the target double-stranded DNA overlaps with the cleavage site determined based on the recognition sequence, in which case the cleavage site is also proximate to the end of that side, and a cleavage is generated at that predetermined position using the restriction nicking enzyme, causing a single-stranded segment of DNA containing the recognition sequence and located between the cleavage site and the end of that side to dissociate from the double strand, generating a single-stranded segment of DNA containing the recognition sequence, and a double-stranded DNA having a double-stranded region and a single-stranded region. Specifically, when the cleavage site of the restriction nicking enzyme is on 3′ side of the recognition sequence, the resulting double-stranded DNA with a double-stranded region and a single-stranded region has a protruding 3′ end, i.e., the single-stranded region is oriented from the nucleotide immediately adjacent to the double-stranded region toward the nucleotide away from the double-stranded region as 5′ to 3′; when the cleavage site of the restriction nicking enzyme is on 5′ side of the recognition sequence, the resulting double-stranded DNA with a double-stranded region and a single-stranded region has a protruding 5′ end, i.e., the single-stranded region is oriented from the nucleotide immediately adjacent to the double-stranded region toward the nucleotide away from the double-stranded region as 3′ to 5′. In some preferred embodiments, a double-stranded DNA fragment containing the restriction nicking enzyme recognition sequence may be added to one end of the double-stranded DNA to be modified to obtain a target double-stranded DNA, said double-stranded DNA fragment is added at a position such that said predetermined position on the target double-stranded DNA overlaps with the cleavage site determined based on the recognition sequence. A cleavage is generated at the predetermined position using the restriction nicking enzyme, a single-stranded segment of DNA containing the recognition sequence and located between the cleavage site and the free end of the added double-stranded DNA fragment is dissociated from the double strand, generating a single-stranded segment of DNA containing the recognition sequence, and a double-stranded DNA having a double-stranded region and a single-stranded region. Persons skilled in the art may be able to understand “proximity” to mean that the distance between a nick and an end is shorter than the distance between the nick and another end, and that the absolute length of the distance is sufficiently small to allow the single strand of DNA between the nick and the end to be dissociated from the double strand. By “proximity”, it means a distance of 5 to 50 bases, preferably 10 to 30 bases, and more preferably 12 to 20 bases from the end to the nick, which is also the length of the single-stranded region of the target double-stranded DNA. In said single cleavage method, although a single nick is created by a single cleavage at a predetermined position on one strand of the target double-stranded DNA to create the single-stranded region, it should be understood that more nicks can be created on one strand of the target double-stranded DNA with one of the nicks at the predetermined position. This can facilitate the speed of cleavage and improve cleavage efficiency when longer single-stranded regions are desired. Multiple nicks can allow for the dissociation of all of the single strands of DNA between the end of one side of the target double-stranded DNA and the nick furthest from that side, which can generate a longer single-stranded region than a single nick.

In some embodiments, a single-stranded region can be generated by two cleavages, i.e., by using restriction nicking enzymes to generate two separate nicks on one strand of the target double-stranded DNA that are close enough to each other to allow a single-stranded segment of DNA between these two nicks to dissociate from the double strand, thereby generating a single-stranded region on the other strand. The dissociation occurs at the appropriate temperature at which the DNA double-strand between these two nicks undergoes denaturation and separation. Said two nicks can be on the same strand or on each of the two strands of double-stranded DNA.

In some particular embodiments, the target double-stranded DNA may contain two recognition sequences of same sequence on the same strand, the two recognition sequences are oriented in the same direction and in close proximity to each other, said predetermined position on the target double-stranded DNA overlaps with the cleavage site determined based on one of the recognition sequences, said restriction nicking enzyme is used to cleave once at each of these two recognition sequence cleavage sites, twice in total, causing a single-stranded segment of DNA containing the recognition sequence and located between the two cleavage sites to dissociate from the double strand, generating a single-stranded segment of DNA containing one recognition sequence, and a double-stranded DNA having a double-stranded region and a single-stranded region, which is referred to as a same direction double-cleavage method. In some preferred embodiments, the target double-stranded DNA can be obtained by adding to the double-stranded DNA to be modified a double-stranded DNA fragment containing two such recognition sequences of same sequence, in the same orientation and in close proximity, said double-stranded DNA fragment is added in such a position that said predetermined position on the target double-stranded DNA overlaps with the cleavage site determined based on one of the recognition sequences, said restriction nicking enzyme is used to cleave once at each of these two recognition sequence cleavage sites, twice in total, causing a single-stranded segment of DNA containing the recognition sequence and located between the two cleavage sites to dissociate from the double strand, generating a single-stranded segment of DNA containing one recognition sequence, and a double-stranded DNA having a double-stranded region and a single-stranded region.

In some other particular embodiments, the target double-stranded DNA may contain two double-stranded DNA fragments of same sequence on each of the two strands, respectively, the two recognition sequences are in opposite directions and in close proximity to each other, said predetermined position on the target double-stranded DNA overlaps with the cleavage site determined based one of the recognition sequences, said restriction nicking enzyme is used to cleave once at each of these two recognition sequence cleavage sites, twice in total, causing the double strand between the two cleavage sites to dissociate, generating two single-stranded regions, which is referred to as an opposite direction double-cleavage method. In some preferred embodiments, the target double-stranded DNA can be obtained by adding to the double-stranded DNA to be modified a double-stranded DNA fragment containing two such recognition sequences of same sequence, in opposite orientation and in relatively close proximity, said double-stranded DNA fragment is added in such a position that said predetermined position on the target double-stranded DNA overlaps with the cleavage site determined based on one of the recognition sequences, said restriction nicking enzyme is used to cleave once at each of these two recognition sequence cleavage sites, twice in total, causing the double strand between the two cleavage sites to dissociate, generating two single-stranded regions.

The above-mentioned method of generating a single-stranded region by double cleavages, such as the same direction double-cleavage method and the opposite direction double-cleavage method can be applied to circular double-stranded DNA, such as double-stranded DNA vectors, e.g., plasmids, etc. Where “proximity” means that the distance between the two nicks is 5 to 50 bases, preferably 10 to 30 bases, more preferably 12 to 20 bases, which is also the length of the single-stranded region of the target double-stranded DNA.

It should be understood that although the above ways of generating a single-stranded region by two cleavages, such as the same direction double-cleavage method and the opposite direction double-cleavage method, generate a single-stranded region by two cleavages, it should be understood that more nicks or cleavages can be generated on one strand of the target double-stranded DNA, with one of the nicks at the predetermined position (same direction double-cleavage method) or two of the nicks at the predetermined positions (opposite direction double-cleavage method). This can facilitate the speed of cleavage and improve cleavage efficiency when longer single-stranded regions are desired. Multiple nicks can allow for the dissociation of all of the DNA single-strands between the two most distant nickes, which can generate longer single-stranded regions than a single nick.

In the above method, “appropriate temperature” refers to the temperature at which said single-stranded region can be generated and the activity of said restriction nicking enzyme can be ensured, typically 37 to 75 degrees Celsius, preferably 45 to 65 degrees Celsius, more preferably 53 to 63 degrees Celsius.

In some embodiments, in the double-stranded DNA fragment added to the double-stranded DNA to be modified, at least one restriction nicking enzyme recognition sequence (which may be two restriction nicking enzyme recognition sequences in the opposite direction double-cleavage method) is immediately adjacent to the end of its cleavage site side, such that, after the addition of said double-stranded DNA fragment, said restriction nicking enzyme recognition sequence is immediately adjacent to the double-stranded DNA to be modified on its cleavage site side. In other embodiments, the double-stranded DNA fragment added to the double-stranded DNA to be modified may have one or more nucleotides between at least one restriction nicking enzyme recognition sequence (which may be two restriction nicking enzyme recognition sequences in the opposite direction double-cleavage method) and the end on its cleavage site side, such that, after the addition of said double-stranded DNA fragment, said restriction nicking enzyme recognition sequence has said one or more nucleotides between its cleavage site side and the double-stranded DNA to be modified, in such a way that the final resulting predetermined end can contain nucleotides otherwise not present in the double-stranded DNA to be modified, i.e., one or more terminal nucleotides are added to the double-stranded DNA to be modified at the same time as the predetermined end is formed, for example, additional nucleotide overhangs can be added to the target double-stranded DNA in this way.

As used herein, the cleavage site side of the restriction nicking enzyme recognition sequence is that side of a defined position relative to said recognition sequence when the restriction nicking enzyme recognizes the recognition sequence and cleaves at the defined position in the vicinity.

The oligonucleotide adapter of the present invention is used to provide the recognition sequence for the restriction nicking enzyme, and a sequence capable of hybridizing to the single-stranded region of the target double-stranded DNA, which by hybridization positions a portion of the nucleotides of the other strand of the target double-stranded DNA in the vicinity of the cleavage site of the recognition site sequence on the oligonucleotide adapter, and a cleavage is performed cleavage at the predetermined position on the other strand of the target double-stranded DNA by the oligonucleotide adapter and the restriction nicking enzyme. The oligonucleotide adapter contains the restriction nicking enzyme recognition sequence in the double-stranded portion, but lacks a sequence cleavable by the restriction nicking enzyme on the cleavage site side of the restriction nicking enzyme recognition sequence and has only its complementary sequence, which constitutes the single-stranded portion of said oligonucleotide adapter, with the end of the double-stranded portion on the cleavage site side of the restriction nicking enzyme recognition sequence being the demarcation point between the double-stranded portion and the single-stranded portion. The single-stranded portion of the oligonucleotide adapter hybridizes with the single-stranded region of the target double-stranded to form a double-stranded structure such that the nucleotides on the other strand of the target double-stranded DNA are in close proximity to said recognition sequence and such that the predetermined position on that other strand is located exactly at the position of the cleavage site on the side of said recognition sequence, thereby enabling said restriction nicking enzyme to cleave at the predetermined position on the other strand of said target double-stranded DNA by recognizing the recognition sequence on the oligonucleotide adapter. Based on the same principle, the oligonucleotide adapter of the present invention can also be used to cleave single-stranded DNA, in this case, the oligonucleotide adapter is used to provide the recognition sequence for the restriction nicking enzyme and a sequence that can hybridize with a predetermined region of the single-stranded DNA, positions the predetermined region of the single-stranded DNA in the vicinity of the cleavage site of the recognition site sequence on the oligonucleotide adapter by hybridization, and a cleavage is performed at the predetermined position on the predetermined region of the single-stranded DNA by the oligonucleotide adapter and the restriction nicking enzyme. The oligonucleotide adapter contains the restriction nicking enzyme recognition sequence in the double-stranded portion, but lacks a sequence cleavable by the restriction nicking enzyme on the cleavage site side of the restriction nicking enzyme recognition sequence and has only its complementary sequence, which constitutes the single-stranded portion of said oligonucleotide adapter, with the double-stranded end on the cleavage site side of the restriction nicking enzyme recognition sequence being the demarcation point between the double-stranded portion and the single-stranded portion. The single-stranded portion of the oligonucleotide adapter hybridizes with the predetermined region of the single-stranded DNA to form a double-stranded structure such that the predetermined region of the single-stranded DNA is in close proximity to said recognition sequence and such that the predetermined position on that predetermined region is located exactly at the position of the cleavage site on the side of said recognition sequence, thereby enabling said restriction nicking enzyme to cleave at the predetermined position on said single-stranded DNA by recognizing the recognition sequence on the oligonucleotide adapter. This type of enzyme digestion of target double-stranded DNA or single-stranded DNA using an oligonucleotide adapter in conjunction with a restriction nicking enzyme is called assisted enzyme digestion.

When the single-stranded portion of the oligonucleotide adapter hybridizes with the single-stranded region of the target double-stranded DNA or with a predetermined region of the single-stranded DNA, the hybridized region extends from the first single-stranded nucleotide in the oligonucleotide adapter immediately adjacent to the double-stranded portion in a direction away from the double-stranded portion. The length of the double-stranded portion of the oligonucleotide adapter can be between 6 to 30 bases, preferably 10 to 15 bases, and the length of its single-stranded portion can be 5 to 50 bases, preferably 10 to 30 bases, more preferably 15 to 20 bases. The length of the region where the single-stranded portion of the oligonucleotide adapter hybridizes with the other strand of the target double-stranded DNA or the single-stranded DNA may be greater than, equal to, or less than the length of its single-stranded portion, which may be, for example, 5 to 100 bases, preferably 10 to 80 bases, more preferably 15 to 70 bases.

In the assisted enzyme digestion method of the present invention, the single-stranded portion of the oligonucleotide adapter and the single-stranded region of the target double-stranded DNA may have different orientations. The orientation of the single-stranded portion of the oligonucleotide adapter and the orientation of the the single-stranded region of the target double-stranded DNA depends on the restriction nicking enzyme used. If the cleavage site of the restriction nicking enzyme used is on 3′ side of its recognition sequence, the single-stranded portion of the corresponding oligonucleotide adapter is oriented from the nucleotide immediately adjacent to the double-stranded portion toward the nucleotide away from the double-stranded portion as 3′ to 5′, and the single-stranded region of the corresponding target double-stranded DNA is oriented from the nucleotide immediately adjacent to the double-stranded region toward the nucleotide away from the double-stranded region as 5′ to 3′. If the cleavage site of the restriction nicking enzyme used is on 5′ side of its recognition sequence, the single-stranded portion of the corresponding oligonucleotide adapter is oriented from the nucleotide immediately adjacent to the double-stranded portion toward the nucleotide away from the double-stranded portion as 5′ to 3′, and the single-stranded region of the corresponding target double-stranded DNA is oriented from the nucleotide immediately adjacent to the double-stranded region toward the nucleotide away from the double-stranded region as 3′ to 5′. In other words, when the single-stranded portion of the oligonucleotide adapter hybridizes with the single-stranded region of the target double-stranded DNA, the nucleotides in the single-stranded portion of the oligonucleotide adapter near the double-stranded portion of the oligonucleotide adapter hybridize with the portion of the single-stranded region of the target double-stranded DNA near the double-stranded region of the target double-stranded DNA, and the nucleotides in the single-stranded portion of the oligonucleotide adapter away from the double-stranded region of the oligonucleotide adapter hybridize with the portion of the single-stranded region of the target double-stranded DNA away from the double-stranded region of the target double-stranded DNA.

According to the above description, it will be clearly understood by those skilled in the art that when two or more cleavages are made on one strand of the target double-stranded DNA using a restriction nicking enzyme, such that a single-stranded segment of DNA containing the recognition sequence and located between the two cleavage sites is dissociated from the double strand, generating a single-stranded segment of DNA containing the recognition sequence, and a double-stranded DNA having a double-stranded region and a single-stranded region, for example, when a double-stranded DNA having a double-stranded region and a single-stranded region is generated by the same direction double-cleavage method, the double-stranded DNA having a double-stranded region and a single-stranded region has a single-stranded region and two double-stranded regions located at the two ends of the single-stranded region, respectively. One and only one of these two double-stranded regions matches the “double-stranded region of the target double-stranded DNA” as described in “when the single-stranded portion of the oligonucleotide adapter hybridizes with the single-stranded region of the target double-stranded DNA, the nucleotides in the single-stranded portion of the oligonucleotide adapter near the double-stranded portion of the oligonucleotide adapter hybridize with the portion of the single-stranded region of the target double-stranded DNA near the double-stranded region of the target double-stranded DNA, and the nucleotides in the single-stranded portion of the oligonucleotide adapter away from the double-stranded region of the oligonucleotide adapter hybridize with the portion of the single-stranded region of the target double-stranded DNA away from the double-stranded region of the target double-stranded DNA”, the end immediately adjacent to the double-stranded region matching that description is the end desired to be manipulated in the next step, in other words, the intended end eventually generated is the end extending from the double-stranded region matching that description. Specifically, when the cleavage position of the restriction nicking enzyme used is on 3′ side of the recognition sequence, the double-stranded region that mathes this description is the double-stranded region immediately adjacent to the 5′ side of the single-stranded region, or the double-stranded region immediately adjacent to the 3′ side of the dissociated DNA single strand; when the cleavage position of the restriction nicking enzyme used is on 5′ side of the recognition sequence, the double-stranded region that matches this description is the double-stranded region immediately adjacent to the 3′ side of the single-stranded region, or the double-stranded region immediately adjacent to the 5′ side of the dissociated DNA single strand. Accordingly, hereinafter, when referring to the double-stranded region of the target double-stranded DNA, unless otherwise specified, for double-stranded DNA having a single-stranded region and two double-stranded regions at each end of the single-stranded region generated by two or more cleavages of the target double-stranded DNA by restriction nicking enzymes, it means the double-stranded region described in this paragraph as matching the description of “when the single-stranded portion of the oligonucleotide adapter hybridizes with the single-stranded region of the target double-stranded DNA, the nucleotides in the single-stranded portion of the oligonucleotide adapter near the double-stranded portion of the oligonucleotide adapter hybridize with the portion of the single-stranded region of the target double-stranded DNA near the double-stranded region of the target double-stranded DNA, and the nucleotides in the single-stranded portion of the oligonucleotide adapter away from the double-stranded region of the oligonucleotide adapter hybridize with the portion of the single-stranded region of the target double-stranded DNA away from the double-stranded region of the target double-stranded DNA”.

In some embodiments, the oligonucleotide adapter may be formed by hybridization of two oligonucleotides, the double-stranded portion is the portion of the two oligonucleotides that hybridize, the single-stranded portion is the portion of the two oligonucleotides that do not participate in the hybridization, with the restriction nicking enzyme recognition site located in the double-stranded portion. In other embodiments, the oligonucleotide adapter may also comprise oligonucleotides that can form a hairpin structure, with the stem of the hairpin comprising a hybridized double-stranded portion and a single-stranded portion (or alternatively, the stem of the hairpin is the double-stranded portion and the open-loop portion is the single-stranded portion), with the restriction nicking enzyme recognition site located in the double-stranded portion of the stem.

In some embodiments, the restriction nicking enzyme recognition sequence may be immediately adjacent to the end of the double-stranded portion on its cleavage site side in said oligonucleotide adapter, and the assisted enzyme digestion using such an oligonucleotide adapter is referred to in the present invention as the immediate adjacent mode. In other embodiments, in the oligonucleotide adapter, there can be 1 nucleotide between the restriction nicking enzyme recognition sequence and the end of the double-stranded portion on its cleavage site side, and the assisted enzyme digestion using such an oligonucleotide adapter is referred to in the present invention as the inter-1 mode. In other embodiments, in the oligonucleotide adapter, there can be 2 nucleotides between the restriction nicking enzyme recognition sequence and the end of the double-stranded portion on its cleavage site side, and the assisted enzyme digestion using such an oligonucleotide adapter is referred to in the present invention as the inter-2 mode. In other embodiments, in the oligonucleotide adapter, there can be 3 nucleotides between the restriction nicking enzyme recognition sequence and the end of the double-stranded portion on its cleavage site side, and the assisted enzyme digestion using such an oligonucleotide adapter is referred to in the present invention as the inter-3 mode. In the present invention, the oligonucleotide adapter is used in conjunction with a restriction nicking enzyme, and the oligonucleotide adapter comprises the recognition site for said restriction nicking enzyme, while the number of nucleotides between the restriction nicking enzyme recognition sequence and the end of the double-stranded portion on its cleavage site side should be less than the number of characteristic nucleotides of the restriction nicking enzyme used. Thus, in other embodiments, the oligonucleotide adapter may also have more than 3 nucleotides between the restriction nicking enzyme recognition sequence and the end of the double-stranded portion on its cleavage site side if the number of characteristic nucleotides of the restriction nicking enzyme used is greater than 4.

The oligonucleotide adapter can be designed such that the restriction nicking enzyme cleaves the other strand of the target double-stranded DNA or single-stranded DNA at a predetermined position. When cleaving the target double-stranded DNA, the position on the other strand of the target double-stranded DNA to be cleaved is determined by a combination of factors, one of which is the number of characteristic nucleotides of the particular restriction nicking enzyme used, another is the number of nucleotides between the recognition sequence and the end of the double-stranded portion on the cleavage site side in the oligonucleotide adapter, and yet another is the position of the hybridization start base of the other strand of the target double-stranded DNA in that other strand. Said hybridization start base is the nucleotide in the hybridization region of the other strand of the target double-stranded DNA (i.e., the sequence that hybridizes to the single-stranded portion of the oligonucleotide adapter) which is immediately adjacent to the double-stranded portion of the oligonucleotide adapter. The position cleaved by the restriction nicking enzyme cleaves is , along the direction from the recognition sequence to the cleavage site, after a specific number of nucleotides from the hybridization start base of the target double-stranded DNA, wherein the specific number is the number of characteristic nucleotides of the particular restriction nicking enzyme used minus the number of bases between the recognition sequence and the end of the double-stranded portion on its cleavage site side in the oligonucleotide adapter. When cleaving the target single-stranded DNA, the factors that determine where on the target single-stranded DNA is cleaved comprise (1) the number of characteristic nucleotides of the particular restriction nicking enzyme used, (2) the number of nucleotides between the recognition sequence and the end of the double-stranded portion on its cleavage site side in the oligonucleotide adapter, and the position of the hybridization start base of the target single-stranded DNA in that single-stranded DNA. The hybridization start base of the target single-stranded DNA referred to herein is the nucleotide in the hybridization region of the target single-stranded DNA (i.e., the sequence that hybridizes to the single-stranded portion of the oligonucleotide adapter) which is immediately adjacent to the double-stranded portion of the oligonucleotide adapter. The position cleaved by the restriction nicking enzyme is, along the direction from the recognition sequence to the cleavage site, after a specific number of nucleotides from the hybridization start base of the target single-stranded DNA, wherein the specific number is the number of characteristic nucleotides of the particular restriction nicking enzyme used minus the number of bases between the recognition sequence and the end of the double-stranded portion on its cleavage site side in the oligonucleotide adapter.

With the assisted enzyme digestion mode, cleavage can be made at a different predetermined position on the other strand of the target double-stranded DNA to generate the desired end, and the cleavage position can be located either on the single-stranded region of the target double-stranded DNA or on the double-stranded region of the target double-stranded DNA.

In some embodiments, the hybridization region of the other strand of the target double-stranded DNA (i.e., the region on the target double-stranded DNA that hybridizes to the single-stranded portion of the oligonucleotide adapter) is located on the single-stranded region and immediately adjacent to its double-stranded region. In other embodiments, the hybridization region of the target double-stranded DNA is located on the single-stranded region and is separated from the double-stranded region by one or more nucleotides, such as one, two or three nucleotides, in this case, assisted enzyme digestion using oligonucleotide adapter can yield 5′ overhangs or 3′ overhangs of different lengths, with the number of overhanging bases being the number of characteristic nucleotides of the restriction nicking enzyme used minus the number of nucleotides between the recognition sequence in and the end on its cleavage site side the oligonucleotide adapter used, then plus the number of nucleotides between the hybridization region and the double-stranded region of the target double-stranded DNA. Whether a 5′ overhang or a 3′ overhang is generated depends on the restriction nicking enzyme used and the corresponding oligonucleotide adapter. If the restriction nicking enzyme used has its cleavage site on 3′ side of its recognition sequence, a 3′ overhang is generated; if the restriction nicking enzyme used has its cleavage site on 5′ side of its recognition sequence, a 5′ overhang is generated. From the above principle, it is clear that the length of the 3′ overhang or 5′ overhang generated by this principle cannot exceed the length of the single-stranded region generated on the target double-stranded DNA at the longest, therefore, if a longer overhang is desired, a longer single-stranded region should be generated on the target double-stranded DNA.

In some particular embodiments, the restriction nicking enzymes used is Nt.AlwI or Nt.BstNBI, both of which have a number of characteristic nucleotides of 4 and have the cleavage site on 3′ side of their recognition sequence. If the number of nucleotides between the recognition sequence and 3′ end of the strand in which it is located in the oligonucleotide adapter is m, and the number of nucleotides separating the hybridization region of the single-stranded region from the double-stranded region of the target double-stranded DNA is n, the number of bases in the resulting 3′ overhang is 4-m+n, provided that m is less than 4. For example, when m=2, a 3′ overhang of 2 bases is generated if the number n of nucleotides separating the hybridization region of the single-stranded region from the double-stranded region of the target double-stranded DNA is 0; a 3′ overhang of 3 bases is generated if the number n of nucleotides separating the hybridization region of the single-stranded region from the double-stranded region of the target double-stranded DNA is 1; a 3′ overhang of 4 bases is generated if the number n of nucleotides separating the hybridization region of the single-stranded region from the double-stranded region of the target double-stranded DNA is 2; a 3′ overhang of 5 bases or more is generated if the number n of nucleotides separating the hybridization region of the single-stranded region from the double-stranded region of the target double-stranded DNA is 3 or more, but in this case, the 3′ overhanging sequence starting from the 5^{th} base of 5′ to 3′ direction comprises the recognition sequence for said restriction nicking enzyme, which is determined by the single-stranded region generation approach of the target double-stranded DNA. Again, for example, when m=0, a 3′ overhang of 4 bases is generated if the number n of nucleotides separating the hybridization region of the single-stranded region from the double-stranded region of the target double-stranded DNA is 0; if the number n of nucleotides separating the hybridization region of the single-stranded region from the double-stranded region of the target double-stranded DNA is 1 or more, a 3′ overhang of 5 bases or more is generated, similarly, in this case, the sequence of the 3′ overhang starting from the 5^{th} base of 5′ to 3′ direction comprises the recognition sequence for said restriction nicking enzyme. Again, for example, when m=1, a 3′ overhang of 3 bases is generated if the number n of nucleotides separating the hybridization region of the single-stranded region from the double-stranded region of the target double-stranded DNA is 0; a 3′ overhang of 4 bases is generated if the number n of nucleotides separating the hybridization region of the single-stranded region from the double-stranded region of the target double-stranded DNA is 1; a 3′ overhang of 5 bases or more is generated, if the number n of nucleotides separating the hybridization region of the single-stranded region from the double-stranded region of the target double-stranded DNA is 2 or more, similarly, in this case, the sequence of the 3′ overhang starting from the 5^{th} base of 5′ to 3′ direction comrises the recognition sequence for said restriction nicking enzyme. For example, when m=3, a 3′ overhang of 1 base is generated if the number n of nucleotides separating the hybridization region of the single-stranded region from the double-stranded region of the target double-stranded DNA is 0; a 3′ overhang of 2 bases is generated if the number n of nucleotides separating the hybridization region of the single-stranded region from the double-stranded region of the target double-stranded DNA is 1; a 3′ overhang of 3 bases is generated if the number n of nucleotides separating the hybridization region of the single-stranded region from the double-stranded region of the target double-stranded DNA is 2; a 3′ overhang of 4 bases is generated if the number n of nucleotides separating the hybridization region of the single-stranded region from the double-stranded region of the target double-stranded DNA is 3; a 3′ overhang of 5 bases or more is generated, if the number n of nucleotides separating the hybridization region of the single-stranded region from the double-stranded region of the target double-stranded DNA is 4 or more, similarly, in this case, the sequence of the 3′ overhang starting from the 5^{th} base of 5′ to 3′ direction comprises the recognition sequence for said restriction nicking enzyme.

In the case where the restriction nicking enzyme used is Nt.AlwI or Nt.BstNBI, when m=2 and n=2, there are only 2 nucleotides in the oligonucleotide adapter that need to hybridize with nucleotides in the final resulting 3′ overhang, i.e., 2 nucleotides starting from the first single-stranded nucleotide of the oligonucleotide adapter immediately adjacent to the double-stranded portion, and thus, except for these oligonucleotides, all other nucleotides on the oligonucleotide adapter are artificially selectable, as long as they fulfill the function of the oligonucleotide adapter. For example, the double-stranded portion of the oligonucleotide adapter is feasible as long as it includes the restriction nicking enzyme recognition site, and the other sequences are not limited, while in the single-stranded portion of the oligonucleotide adapter, the nucleotides other than the said two nucleotides described need to be able to hybridize with the corresponding portion of the single-stranded region of the target double-stranded DNA, while this part of the single-stranded region of the target double-stranded DNA can be artificially added to the double-stranded DNA to be modified, and thus its sequence can be not limited (except for the complementary sequence of the restriction nicking enzyme recognition sequence contained therein). Thus, with the same restriction nicking enzymes used, the nucleotides in the oligonucleotide adapter other than the said two nucleotides can be fixed (which means that the sequence of the double-stranded DNA fragment containing the restriction nicking enzyme recognition sequence and added to the target double-stranded DNA is also fixed), and 16 oligonucleotide adapters are prepared according to all permutations of the the said two nucleotides, wherein each oligonucleotide adapter contains one permutation of the the said two nucleotides and 16 oligonucleotide adapters contain all 16 permutations of the the said two nucleotides. A mixture of these oligonucleotide adapters can be used as universal oligonucleotide adapters suitable for assisted enzyme digestion of a variety of different target double-stranded DNAs with n=2.

In some other particular embodiments, the region on the target double-stranded DNA that hybridizes to the single-stranded portion of the oligonucleotide adapter includes not only the single-stranded region of that target double-stranded DNA, but also a portion of the double-stranded region sequence adjacent to that single-stranded region, in other words, the hybridization start base of the target double-stranded DNA is located on the double-stranded portion of the target double-stranded DNA. In this case, the single-stranded portion of the oligonucleotide adapter includes not only a sequence capable of hybridizing with the single-stranded region of the target double-stranded DNA, but also included between this sequence and the double-stranded portion of the oligonucleotide adapter a sequence capable of hybridizing to a segment in the double-stranded region of the target double-stranded DNA adjacent to the single-stranded region. This type of assisted enzyme digestion is referred to in the present invention as invasive assisted enzyme digestion, and the sequence in the single-stranded portion of the oligonucleotide adapter that can hybridize with a segment in the double-stranded region of the target double-stranded DNA adjacent to the single-stranded region is referred to as the “invading region”, the segment of the double-stranded region adjacent to the single-stranded region is called the “invaded region”. The invaded region is originally double-stranded, and during the hybridization of the oligonucleotide adapter with the target double-stranded DNA, the sequence of the invaded region may undergo double-stranded dissociation and hybridize with the invading region. The invading region is immediately adjacent to the double-stranded portion of the oligonucleotide adapter at one end and to a sequence in the single-stranded portion of the oligonucleotide adapter that can hybridize with the single-stranded region of the target double-stranded DNA at one end. The length of the invading and invaded regions can be between 1 to 100 bases, preferably between 1 to 30 bases, and more preferably between 3 to 20 bases.

Invasive assisted enzyme digestion using oligonucleotide adapters can result in 3′ overhangs of different lengths, blunt ends, or 5′ overhangs of different lengths. A 5′ or 3′ overhang is generated when the number of characteristic nucleotides of the restriction nicking enzyme used minus the number of nucleotides separating the recognition sequence from the end on its cleavage site side in the oligonucleotide adapter used is greater than the number of nucleotides in the invading region, with the length of the overhang being the number of characteristic nucleotides of the restriction nicking enzyme used minus the number of nucleotides separating the recognition sequence from the ends on its cleavage site side in the oligonucleotide adapter used, then minus the number of nucleotides in the invading region. In this case, whether a 5′ overhang or a 3′ overhang is generated depends on the restriction nicking enzyme used and the corresponding oligonucleotide adapter, if the cleavage site of the restriction nicking enzyme used is on 3′ side of its recognition sequence, a 3′ overhang is generated; if the cleavage site of the restriction nicking enzyme used is on 5′ side of its recognition sequence, a 5′ overhang is generated.

A blunt end is generated when the number of characteristic nucleotides of the restriction nicking enzyme used minus the number of nucleotides separating the recognition sequence from the end on its cleavage site side in the oligonucleotide adapter used equals the number of nucleotides in the invading region. In this case, the blunt end is generated regardless of whether the cleavage site of the restriction nicking enzyme used is on 3′ side of its recognition sequence or on 5′ side of its recognition sequence.

A 5′ overhang or 3′ overhang is generated when the number of characteristic nucleotides of the restriction nicking enzyme used minus the number of nucleotides separating the recognition sequence used from the end on its cleavage site side in the oligonucleotide adapter is less than the number of nucleotides in the invading region, the length of the overhang is the difference obtained by the number of nucleotides in the invading region minus a number obtained by subtracting the number of nucleotides spaced between the recognition sequence and the end of its cleavage site side in the oligonucleotide adapter used from the number of characteristic nucleotides of the restriction nicking enzyme used. In this case, whether a 5′ overhang or a 3′ overhang is generated depends on the restriction nicking enzyme used and the corresponding oligonucleotide adapter, if the cleavage site of the restriction nicking enzyme used is on 3′ side of its recognition sequence, a 3′ overhang is generated; if the cleavage site of the restriction nicking enzyme used is on 5′ side of its recognition sequence, a 5′ overhang is generated.

In some particular embodiments, the restriction nicking enzymes used is Nt.AlwI or Nt.BstNBI, both of which have a number of characteristic nucleotides of 4 and whose cleavage sites are on 3′ side of the recognition sequence. If the number of nucleotides between the recognition sequence and 3′ end of the strand in which it is located in the oligonucleotide adapter is m and the number of nucleotides in the invading region is i, then when 4-m >i, a 3′ overhang is generated and the length of the 3′ overhang is 4-m-i; when 4-m=i, a blunt end is generated; and when 4-m <i, a 5′ overhang is generated and the length of the 3′ overhang is i-(4-m). For example, in the case of m=2, a 3′ overhang of 2 bases is generated if i=0, a 3′ overhang of 1 base if i=1, a blunt end is generated if i=2, and a 5′ overhang of length i−2 is generated if i >2.

The present inventors further found that various restriction cleavage enzymes allow the recognition sequence to be located on a different DNA molecule than the cleaved sequence, and thus manipulation of double-stranded DNA ends can be achieved using all types of restriction nicking enzymes and the corresponding oligonucleotide adapters. The first step is to use restriction nicking enzymes to first generate one or more nicks on one strand of double-stranded DNA to generate a single-stranded region on the other strand. The second step is to use hybridization of an oligonucleotide adapter with the single-stranded region and then combine it with the same or a different restriction nicking enzyme to generate a cleavage on the other strand of the DNA double-strand in conjuction, the position of cleavage correlates with the sequence selection of the oligonucleotide adapter, which ultimately cleaves the target double-stranded DNA and generates ends of customizable length and customizable hanging species at the cleavage. The restriction nicking enzyme used to generate the single-stranded region may be referred to in the present invention as a first restriction nicking enzyme, and the restriction nicking enzyme used to cleave the other strand may be referred to in the present invention as a second restriction nicking enzyme, and the first restriction nicking enzyme and the second restriction nicking enzyme may be the same or different.

Thus, a second aspect of the present invention provides a method of generating a predetermined double-stranded DNA end, said method using different restriction nicking enzymes, one restriction nicking enzyme for generating a single-stranded region on the target double-stranded DNA and another restriction nicking enzyme for cleaving the other strand of the target double-stranded DNA in the presence of an oligonucleotide adapter to generate the predetermined end.

Said method of generating a predetermined double-stranded DNA end comprises:

generating one or more nicks at a predetermined position on one strand of a target double-stranded DNA using a first restriction nicking enzyme to generate a single-stranded region on the other strand of the target double-stranded DNA;

using an oligonucleotide adapter having a recognition site for a second restriction nicking enzyme to hybridize with said single-stranded region in combination with use of the second restriction nicking enzyme to generate a cleavage at a predetermined position on the other strand of the target double-stranded DNA, eventually cleaving the target double-stranded DNA and generating the predetermined end at the cleavage site;

wherein said oligonucleotide adapter is a DNA molecule having a double-stranded portion and a single-stranded portion, said oligonucleotide adapter comprises the recognition site for said second restriction nicking enzyme and further comprises a complementary sequence to the cleavage site of said second restriction nicking enzyme but lacks a sequence that can be cleaved by said second restriction nicking enzyme, the single-stranded portion of said oligonucleotide adapter hybridizes with the single-stranded region of the target double-stranded DNA and forms, together with the double-stranded portion of said oligonucleotide adapter, a double-stranded structure recognizable and cleavable by said second restriction nicking enzyme, and thereby leaves the predetermined position on the other strand of the target double-stranded DNA in a position that can be cleaved by said restriction nicking enzyme by recognition of the recognition site on the oligonucleotide adapter by said second restriction nicking enzyme;

said first restriction nicking enzyme is the same as or different from said second restriction nicking enzyme.

Said “complementary sequence of the cleavage site” means that the sequence is complementary to a sequence containing the complete cleavage site.

Persons skilled in the art can appreciate that the single-stranded region of the target double-stranded DNA hybridizes with the single-stranded portion of the oligonucleotide adapter to form a hybridized region immediately adjacent to the double-stranded portion of the oligonucleotide adapter, whereby said restriction nicking enzyme recognizes the recognition site on said double-stranded portion of the oligonucleotide adapter and cleaves the other strand of the target double-stranded DNA at the predetermined position.

The predetermined position on the other strand of the target double-stranded DNA being cleaved can be located in a single-stranded region on the other strand of the target double-stranded DNA or in a double-stranded region on the other strand of the target double-stranded DNA, which can be achieved by the design of the oligonucleotide adapter.

In the method provided by the second aspect of the present invention, unless otherwise stated, the definitions and descriptions of the respective terms are the same as in the method provided by the first aspect described previously.

In the method provided by the second aspect of the present invention, said “predetermined end” includes 3′ overhangs of different lengths, blunt ends, or 5′ overhangs of different lengths. In the present invention, the term “overhang” refers to the presence of unpaired single-stranded nucleotides at the ends of double-stranded DNA, and the length of the overhang is the number of such unpaired single-stranded nucleotides. 3′ overhang means that the end of the overhanging base away from the double strand is the 3′ end, which can also be referred to as the 3′ protruding sticky end. 5′ overhang means that the end of the overhanging base away from the double strand is the 5′ end, which can also be referred to as the 5′ protruding sticky end. The length of the 3′ overhang or 5′ overhang obtained by the method of the present invention can be tailored to be between 0 and 50 bases, preferably 0 to 20 bases, more preferably 2 to 10 bases. Overhanging bases can be used for hybridization or ligation with other double-stranded DNA ends or single-stranded DNA, or as probes.

In the method provided in the second aspect of the present invention, “nicking” or “incising” refers to cleaving only one strand of double-stranded DNA, therefore, in the present invention, “nicking” can be used interchangeably with “cleaving” of one strand of double-stranded DNA, and “cleavage site”, “cleavage position”, “enzyme digestion site” and “enzyme digestion position” are used interchangeably. Said “one or more nicks” can mean one, two, three, four or more nicks. Restriction nicking enzymes, which can also be referred to as restriction incisioin enzymes, nicking endonucleases, or incision endonucleases, are DNA endonucleases that recognize a specific sequence and cleave only one strand of double-stranded DNA at a defined position near the recognized sequence.

Regarding Restriction Nicking Enzymes

The first restriction nicking enzyme may be a restriction nicking enzyme whose recognition sequence does not overlap with the cleavage position or a restriction nicking enzyme whose recognition sequence overlaps with the cleavage position; the second restriction nicking enzyme is preferably a restriction nicking enzyme whose recognition sequence does not overlap with the cleavage position.

The first restriction nicking enzyme and the second restriction nicking enzyme may be the same or different, for example, in some embodiments, the first restriction nicking enzyme and the second restriction nicking enzyme are the same restriction nicking enzyme whose recognition sequence does not overlap with the cleavage position; in other embodiments, the first restriction nicking enzyme and the second restriction nicking enzyme are both restriction nicking enzymes whose recognition sequence does not overlap with the cleavage position, but are different; in yet other embodiments, the first restriction nicking enzyme is any of the restriction nicking enzymes whose recognition sequence overlaps with the cleavage position and the second restriction nicking enzyme is any of the restriction deletion nicking enzymes whose recognition sequence does not overlap with the cleavage position.

Said restriction nicking enzyme whose recognition sequence does not overlap with the cleavage position may be a restriction nicking enzymes whose recognition sequence itself does not overlap with the cleavage position, for example, a restriction nicking enzyme whose recognition sequence itself is immediately adjacent to the cleavage position, or whose recognition sequence itself is separated from the cleavage position by 1, 2, 3, 4, 5 or more nucleotides, also a restriction nicking enzyme whose recognition sequence is immediately adjacent to the cleavage position, and may also be a restriction nicking enzyme in which the complementary sequence of the recognition sequence on the minus strand does not overlap with the cleavage position, for example, a restriction nicking enzyme in which the complementary sequence of the recognition sequence on the minus strand is immediately adjacent to the cleavage position, or in which the complementary sequence of the recognition sequence on the minus strand is separated from the cleavage position by 1, 2, 3, 4, 5 or more nucleotides. Such restriction nicking enzymes include but are not limited to: Nt.AlwI, Nt.BsmAI, Nt.BspQI, Nb.BsrDI, Nt.BstNBI, Nb.BtsI, preferably Nt.BstNBI and Nt.AlwI, more preferably Nt.BstNBI. The recognition sequences of these restriction nicking enzymes and their cleavage sites are well known to those skilled in the art, for example, Nt.BstNBI recognizes 5′-GAGTC-3′ and cleaves between the 4^{th} and 5^{th} base after GAGTC, Nt.AlwI recognizes 5′-GGATC-3′ and cleaves between the 4^{th} and 5^{th} bases after GGATC, Nt.BsmAI recognizes 5′-GTCTC-3′ and cleaves between the 1^{st} and 2^{nd} bases after GTCTC, Nt.BspQI recognizes 5′-GCTCTTC-3′ and cleaves between the 1^{st} and 2^{nd} bases after GCTCTTC, Nb.BtsI recognizes 5′-GCAGTG-3′ and cleaves the 5′ end of the complementary sequence of the recognition sequence 3′-CGTTAC-5′ on the minus strand at a position immediately adjacent to the complementary sequence, and Nb.BsrDI recognizes 5′-GCAATG-5′ and cleaves the 5′ end of the complementary sequence 3′-CGTCAC-5′ of the recognition sequence on the minus strand at a position immediately adjacent to the complementary sequence.

The restriction nicking enzyme whose recognition sequence overlaps the cleavage position may be a restriction nicking enzyme whose cleavage position is inside the recognition sequence, or may be a restriction nicking enzyme whose cleavage position is inside the complementary sequence of the recognition sequence on the minus strand; such a restriction nicking enzyme may be, for example, Nt.BbvCI, Nb.BbvCI, Nb.BsmI, or Nb.BssSI. The recognition sequences of these restriction nicking enzymes and their cleavage sites are well known to those skilled in the art, for example, Nt.BbvCI recognizes 5′-CCTCAGC-3′ and cleaves between CC and TCAGC in this recognition sequence, and Nb.BbvCI recognizes 5′-CCTCAGC-3′ and cleaves between GGAGT and CG in the complementary sequence 3′-GGAGTCG-5′ of this recognition sequence on the minus strand, Nb.BsmI recognizes 5′-GAATGC-3′ and cleaves the position between CTTAC and G in the complementary sequence 3′-CTTACG-5′ of this recognition sequence on the minus strand, and Nb.BssSI recognizes 5′-CACGAG-3′ and cleaves the position between GTGCT and C in the complementary sequence 3′-GTGCTC-5′ of this recognition sequence on the minus strand.

“Recognition sequence” or “recognition sequence of a restriction nicking enzyme” means a specific sequence that is recognized by a restriction nicking enzyme and in the vicinity of which one strand of double-stranded DNA is cleaved at a defined position. For a particular restriction nicking enzyme, the recognition sequence is well defined and persons skilled in the art can easily be informed of the known recognition sequences of restriction nicking enzymes, for example by searching through the website https://international.neb.com/products/restriction-endonucleases/hf-nicking -master-mix-time-s aver-other/nicking-endonucleases/nicking-endonucleases .

In the present invention, according to the existing known restriction nicking enzymes, restriction nicking enzymes include at least three classes, the first being restriction nicking enzymes in which the cleavage position is on the same strand as the recognition sequence and on 3′ side of the recognition sequence, and in which the recognition sequence itself is separated from the cleavage position by 1, 2, 3, 4, 5 or more nucleotides, which may be referred to in the present invention as the first class of restriction nicking enzymes, including Nt.BstNBI, Nt.AlwI, Nt.BsmAI and Nt.BspQI; the second being restricition nicking enzymes in which the cleavage position is not on the same strand as the recognition sequence and located immediately adjacent to the complementary sequence of the recognition sequence on the minus strand on 5′ side of complementary sequence, and which may be referred to in the present invention as the second class of restriction nicking enzymes, include Nb.BtsI and Nb.BsrDI; and the third being restriction nicking enzymes whose cleavage position is inside the recognition sequence, or whose cleavage position is inside the complementary sequence of the recognition sequence on the minus strand, which may be referred to in the present invention as the third class of restriction nicking enzymes, includes Nt.BbvCI, Nb.BbvCI, Nb.BsmI, or Nb.BssSI. Thus, the first restriction nicking enzyme may be any of the first to third classes of restriction nicking enzymes, and the second restriction nicking enzyme may be any of the first or second classes of restriction nicking enzymes. The method of the present invention includes the use of a combination of any one of the first restriction nicking enzymes and any one of the second restriction nicking enzymes.

While the first restriction nicking enzyme and the second restriction nicking enzyme are different, more options are available for the first restriction nicking enzyme, for example, a restriction nicking enzyme with a longer recognition sequence can be selected so that its recognition sequence has a lower chance of appearing on the target DNA to reduce the damage to the integrity of the target DNA double-strand by the nicking enzyme, such as Nt.BspQI or Nb.BbvCI, whose recognition sequence is more than 6 bases in length; while the second restriction nicking enzyme can be a restriction nicking enzyme with a slightly shorter recognition sequence. In some particular embodiments, the second restriction nicking enzyme used to cleave the other strand is Nt.AlwI or Nt.BstNBI, while the first restriction nicking enzyme used to generate the single-stranded region is Nt.BspQI, Nb.BbvCI or Nt.BbvCI. The latter has a longer recognition sequence than Nt.BstNBI and thus has a lower probability of occurrence in the target double strand, which in turn better ensures the integrity of the target DNA double-strand.

The above restriction nicking enzymes contain “Nt” or “Nb” in their names, and the restriction nicking enzymes containing “Nt” in their names are the restriction nicking enzymes of the Nt series, and the restriction nicking enzymes containing “Nb” in their names are the restriction nicking enzymes of the Nb series, and the recognition sequences of the restriction nicking enzymes of the Nt series and their cleavage sites are on the same strand of double-stranded DNA, and the recognition sequences of the restriction nicking enzymes of the Nb series and their cleavage sites are on different strands of double-stranded DNA. The recognition sequence and its cleavage site on the same strand of double-stranded DNA as described in the present invention means that both the recognition sequence and the cleavage site are located on the plus strand of double-stranded DNA, or both are located on the minus strand of double-stranded DNA, and it is not required that the recognition sequence and the cleavage site are located on the same continuous strand, and they can be disconnected from each other. By “disconnected” in the present invention, it means that the two nucleotides on either side of the breakpoint are immediately adjacent but not covalently linked.

The inventors have found that when cleaving double-stranded DNA using restriction nicking enzymes, a break at a position other than the recognition sequence and the cleavage site does not affect the cut; for example, when the cleavage site and the recognition sequence are located on the same strand of the double-stranded DNA but do not overlap each other, a break can be made at a position between the cleavage site and the recognition sequence, and when the cleavage site and the recognition sequence are each located on a different strand of the double-stranded DNA, a break can be made between the cleavage site and the sequence adjacent to it, and such a the break will not affect the cleavage as long as the portion of the double-stranded DNA containing the complete recognition sequence and the complete cleavage site remains in the fully complementary hybridized state of the double strand. Based on this finding, the inventors proposed that an oligonucleotide adapter can be used to provide the recognition sequence, a single-stranded region on the target double-stranded DNA can be used to provide the cleavage site, the recognition sequence of the restriction nicking enzyme can be placed on the suitable position of the oligonucleotide adapter, and the single-stranded portion of the oligonucleotide adapter can hybridize with the single-stranded region of the target double-stranded DNA so that a predetermined position on the target double-stranded DNA is exactly at the position where the restriction nicking enzyme can cleave by recognizing the recognition sequence on the oligonucleotide adapter, a cleavage at the predetermined position on the target double-stranded DNA is thus achieved, resulting in predetermined ends of different types and lengths. Even if the cleavage site is contained in another sequence, and a portion of that sequence is unable to hybridize with the single-stranded portion of the oligonucleotide adapter, cleavage at said cleavage site can be achieved as long as the portion of the sequence containing the complete cleavage site therein can hybridize with the oligonucleotide adapter to form a fully complementary double strand. Said “complete complementary” means that there are no gaps in the hybrid duplex and for each nucleotide there is a nucleotide that hybridizes with it. It should be understood by those skilled in the art that if cleavage with a restriction nicking enzyme is to be achieved, in a complementary hybrid duplex containing a recognition sequence and a cleavage site, both the recognition sequence and the cleavage site should be intact, with the intact recognition sequence meaning that the nucleotides of the recognition sequence are covalently linked to each other and the intact cleavage site meaning that the nucleotides on either side of the cleavage site are covalently linked.

For restriction nicking enzymes in which the recognition sequence does not overlap with the cleavage position, the number of nucleotides between the recognition sequence and the cleavage position is referred to in the present invention as the number of characteristic nucleotides of that restriction nicking enzyme.

Regarding Target Double-Stranded DNA

In the method provided in the second aspect of the present invention, the target double-stranded DNA may refer to double-stranded DNA containing a recognition site for a first restriction nicking enzyme and which can be cleaved by said first restriction nicking enzyme to generate one or more nicks at predetermined positions on one strand thereof, or double-stranded DNA with a single-stranded region resulting from such target double-stranded DNA being cleaved by the first restriction nicking enzyme. When some double-stranded DNA needs to be modified to have predetermined ends, these double-stranded DNAs do not necessarily have the recognition site for the first restriction nicking enzyme and may not be directly usable as said target double-stranded DNA. Thus, in some embodiments, a double-stranded DNA fragment containing the first restriction nicking enzyme recognition sequence is added to the double-stranded DNA to be modified to obtain target double-stranded DNA, the cleavage site determined by the recognition sequence is located on the double-stranded DNA to be modified, said double-stranded DNA fragment is added at a position such that said first restriction nicking enzyme is capable of generating one or more nicks at a predetermined position on one strand of the target DNA double-strand by recognizing the added recognition sequence and thereby generating a single-stranded region on the other strand.

In the method provided in the second aspect of the present invention, the double-stranded DNA to be modified is the double-stranded DNA that needs to be modified to obtain a predetermined end. The double-stranded DNA to be modified can be double-stranded DNA that needs to be spliced, and since performing splicing, especially seamless splicing, requires double-stranded DNA with specific ends, specific ends need to be generated on these double-stranded DNA. The double-stranded DNA to be modified can be linear or circular double-stranded DNA obtained by any method, such as linear double-stranded DNA obtained by PCR, or vector double-stranded DNA, such as a plasmid.

If the double-stranded DNA sequence to be modified happens to have the recognition site of said first restriction nicking enzyme at a suitable position, the double-stranded DNA to be modified can be directly used as the target double-stranded DNA. However, since the probability of occurrence of the first restriction nicking enzyme recognition site on random double-stranded DNA is small, in most cases, the target double-stranded DNA is obtained by adding a double-stranded DNA fragment containing the first restriction nicking enzyme recognition sequence to the double-stranded DNA to be modified. By adding a double-stranded DNA fragment containing the first restriction nicking enzyme recognition sequence, it is possible to generate a nick at any predetermined position on the double-stranded DNA to be modified, without the restriction of the original recognition sequence in the double-stranded DNA to be modified.

In some embodiments, for linear target double-stranded DNA, the single-stranded region can be generated by a single cleavage, i.e., using the first restriction nicking enzyme to generate a nick at the predetermined position on one strand of the target double-stranded DNA that is close to the end of one side of the target double-stranded DNA, causing the single-stranded segment of DNA between the nick and the end of that side to dissociate from the double strand, thereby generating a single-stranded region on the other strand. The dissociation occurs at the appropriate temperature at which the DNA double-strand between the nick and the end undergoes denaturation and separation. This approach is known as the single cleavage method in the present invention and can be applied when the double-stranded DNA to be modified is linear, such as PCR products, and the like.

In the single cleavage method, the target double-stranded DNA contains the recognition sequence of said first restriction nicking enzyme at a position close to the end of one of its sides, said predetermined position on the target double-stranded DNA overlaps with the cleavage site determined according to this recognition sequence, in which case the cleavage site is also close to that side end, and a cleavage is generaed at this predetermined position using the first restriction nicking enzyme, causing the single strand of DNA between the cleavage site and the end of this side to dissociate from the double strand, generating a single-stranded DNA fragment, and a double-stranded DNA having a double-stranded region and a single-stranded region. The structure of the resulting double-stranded DNA with a double-stranded region and a single-stranded region should be compatible with the second restriction nicking enzyme used. Specifically, when the second restriction nicking enzyme is the first class restriction nicking enzyme (e.g., Nt.BstNBI, Nt.AlwI, Nt.BspQI, or Nt.BsmAI), for use in conjuction with the second restriction nicking enzyme and the corresponding oligonucleotide adapter, the double-stranded DNA with a double-stranded region and a single-stranded region resulting from cleavage of the target double-stranded DNA by the first restriction nicking enzyme preferably has a protruding 3′ end, i.e., the single-stranded region is oriented from the nucleotide immediately adjacent to the nucleotide of the double-stranded region to the nucleotide away from the double-stranded region as 5′ to 3′. When the second restriction nicking enzyme is the second class restriction nicking enzyme (e.g., Nt.BstNBI or Nt.AlwI), for use in conjuction with the second restriction nicking enzyme and the corresponding oligonucleotide adapter, the double-stranded DNA with a double-stranded region and a single-stranded region resulting from cleavage of the target double-stranded DNA by the first restriction nicking enzyme preferably has a protruding 5′ end, i.e., the single-stranded region is oriented from the nucleotide immediately adjacent to the nucleotide of the double-stranded region to the nucleotide away from the double-stranded region as 3′ to 5′. It should be understood by those skilled in the art that this can be achieved by the selection and design of the recognition sequence of the first restriction nicking enzyme.

In some preferred embodiments, a double-stranded DNA fragment containing the first restriction nicking enzyme recognition sequence may be added to one end of the double-stranded DNA to be modified to obtain the target double-stranded DNA, said double-stranded DNA fragment is added at a position such that said predetermined position on the target double-stranded DNA overlaps with the cleavage site determined according to the recognition sequence, the first restriction nicking enzyme is used to cleave at the predetermined position, a single-stranded segment of DNA between the cleavage site and the free end of the added double-stranded DNA fragment is dissociated from the double strand, generating a single-stranded DNA fragment, and a double-stranded DNA having a double-stranded region and a single-stranded region.

A person of skill in the art can understand “proximity” to mean that the distance between a nick and one end is closer than the distance between the nick and the other end, and that the absolute length of the distance is small enough to allow the single strand of DNA between the nick and the end to be dissociated from the double strand. By “proximity”, it means a distance of 5 to 50 bases, preferably 10 to 30 bases, and more preferably 12 to 20 bases from the nick to the end, which is also the length of the single-stranded region of the target double-stranded DNA.

In said single cleavage method, although a single nick is generated by a single cleavage at the predetermined position on one strand of the target double-stranded DNA to generate a single-stranded region, it should be understood that more nicks can be generated on one strand of the target double-stranded DNA, with one of the nicks at the predetermined position, this method is also referred to as a single cleavage method. Multiple nicks can cause the entire single strand of DNA between the end of one side of the target double-stranded DNA and the nick farthest from the end of this side to dissociate, which can generate a longer single-stranded region than a single nick. This can facilitate enzyme digestion speed and improve cleavage efficiency when longer single-stranded regions are desired.

In some embodiments, a single-stranded region can be generated by two cleavages, i.e., using the first restriction nicking enzyme to generate two separate nicks on one strand of the target double-stranded DNA that are close enough to each other to allow a single-stranded segment of DNA between these two nicks to dissociate from the double strand, thereby generating a single-stranded region on the other strand. The dissociation occurs at the appropriate temperature at which the DNA double-strand between these two nicks undergoes denaturation and separation. Said two nicks can be on the same strand or on each of the two strands of double-stranded DNA.

In some particular embodiments, the target double-stranded DNA may contain two first restriction nicking enzyme recognition sequences of same sequence on the same strand, the two recognition sequences are oriented in the same direction and in close proximity to each other, said predetermined position on the target double-stranded DNA overlaps with the cleavage site determined according to one of the recognition sequences, said first restriction nicking enzyme is used to cleave once at each of these two recognition sequence cleavage sites, twice in total, causing a single-stranded segment of DNA between the two cleavage sites to dissociate from the double strand, generating a single-stranded segment of DNA, and a double-stranded DNA having a double-stranded region and a single-stranded region, which is referred to as a same direction double-cleavage method. In some preferred embodiments, the target double-stranded DNA can be obtained by adding to the double-stranded DNA to be modified a double-stranded DNA fragment containing two such first restriction nicking enzyme recognition sequences of same sequence, in the same orientation, and in close proximity, said double-stranded DNA fragment is added in such a position that said predetermined position on the target double-stranded DNA overlaps with the cleavage site determined based on one of the recognition sequences, said restriction nicking enzyme is used to cleave once at each of these two recognition sequence cleavage sites, twice in total, causing a single-stranded segment of DNA b5etween the two cleavage sites to dissociate from the double strand, generating a single-stranded segment of DNA, and a double-stranded DNA having a double-stranded region and a single-stranded region.

In some other particular embodiments, two strands of target double-stranded DNA that can be complementary to each other each contain two recognition sequences of the first restriction nicking enzyme with identical sequences, the two recognition sequences are in opposite directions and in close proximity to each other, said predetermined position on the target double-stranded DNA overlaps with the cleavage site determined based one of the recognition sequences, said first restriction nicking enzyme is used to cleave once at each of these two recognition sequence cleavage sites, twice in total, causing the double strand between the two cleavage sites to dissociate, generating two single-stranded regions, which is referred to as a opposite direction double-cleavage method. In some preferred embodiments, the target double-stranded DNA can be obtained by adding to the double-stranded DNA to be modified a double-stranded DNA fragment containing two such first restriction nicking enzyme recognition sequences of same sequence, in opposite orientation and in relatively close proximity, said double-stranded DNA fragment is added in such a position that said predetermined position on the target double-stranded DNA overlaps with the cleavage site determined based on one of the recognition sequences, said first restriction nicking enzyme is used to cleave once at each of these two recognition sequence cleavage sites, twice in total, causing the double strand between the two cleavage sites to dissociate, generating two single-stranded regions.

The above-mentioned generation of single-stranded regions by two cleavages, such as the same direction double-cleavage method and the opposite direction double-cleavage method can be applied to circular double-stranded DNA, such as double-stranded DNA vectors, e.g. plasmids. Where “proximity” means that the distance between the two nicks is 5 to 50 bases, preferably 10 to 30 bases, more preferably 12 to 20 bases, which is also the length of the single-stranded region of the target double-stranded DNA.

It should be understood that although the above ways of generating a single-stranded region by two cleavages, such as the same direction double-cleavage method and the opposite direction double-cleavage method, generate a single-stranded region by two cleavages, it should be understood that more nicks or cleavages can be generated on one strand of the target double-stranded DNA, with one of the nicks at the predetermined position (same direction double-cleavage method) or two of the nicks at the predetermined positions (opposite direction double-cleavage method). Multiple nicks can allow for the dissociation of all of the DNA single-strands between the two most distant nickes, which can generate longer single-stranded regions than two nicks, and this cleavage method is also included in the same direction double-cleavage method and the opposite direction double-cleavage method described in the present invention, respectively. This can facilitate the speed of cleavage and improve cleavage efficiency when longer single-stranded regions are desired.

In the above method, the term “appropriate temperature” refers to the temperature at which said single-stranded region can be generated and said restriction nicking enzyme activity can be ensured, typically 37 to 75 degrees Celsius, preferably 45 to 65 degrees Celsius, more preferably 53 to 63 degrees Celsius.

In some embodiments, when the first restriction nicking enzyme is a restriction nicking enzyme whose recognition sequence does not overlap with the cleavage position, in the double-stranded DNA fragment added to the double-stranded DNA to be modified, at least one first restriction nicking enzyme recognition sequence (which may be two first restriction nicking enzyme recognition sequences in the opposite direction double-cleavage method) is immediately adjacent to the end of its cleavage site side, such that, after the addition of said double-stranded DNA fragment, said first restriction nicking enzyme recognition sequence is immediately adjacent to the double-stranded DNA to be modified on its cleavage site side. In other embodiments, the double-stranded DNA fragment added to the double-stranded DNA to be modified may have one or more nucleotides between at least one first restriction nicking enzyme recognition sequence (which may be two first restriction nicking enzyme recognition sequences in the opposite direction double-cleavage method) and the end on its cleavage site side, such that, after the addition of said double-stranded DNA fragment, said first restriction nicking enzyme recognition sequence has said one or more nucleotides between its cleavage site side and the double-stranded DNA to be modified, in such a way that the final resulting predetermined end can contain nucleotides otherwise not present in the double-stranded DNA to be modified, i.e., one or more terminal nucleotides are added to the double-stranded DNA to be modified at the same time as the predetermined end is formed, for example, additional nucleotide overhangs can be added to the target double-stranded DNA in this way.

In the method provided in the second aspect of the present invention, the cleavage site side of the first restriction nicking enzyme recognition sequence is that side of a defined position relative to said recognition sequence when the restriction nicking enzyme recognizes the recognition sequence and cleaves at the defined position in the vicinity, in the case where the first restriction nicking enzyme is a restriction nicking enzyme whose recognition sequence does not overlap with the cleavage position,

Oligonucleotide Adapters

In the method provided in the second aspect of the present invention, the oligonucleotide adapter is used to provide the recognition sequence for the second restriction nicking enzyme, and a sequence capable of hybridizing to a single-stranded region of the target double-stranded DNA, which by hybridization positions a portion of the nucleotides of the other strand of the target double-stranded DNA in the vicinity of the cleavage site of the recognition site sequence on the oligonucleotide adapter, and a cleavage is performed at the predetermined position on the other strand of the target double-stranded DNA by the oligonucleotide adapter and the second restriction nicking enzyme.

The oligonucleotide adapter includes a double-stranded portion and a single-stranded portion. In some embodiments, the oligonucleotide adapter may be formed by hybridization of two oligonucleotide chains, the double-stranded portion is the portion of the two oligonucleotides that hybridize, the single-stranded portion is the portion of the two oligonucleotides that do not participate in the hybridization. In other embodiments, the two chains that hybridize to form the oligonucleotide adapter are linked at one end to form a hairpin structure, which can also be considered as the oligonucleotide adapter being composed of an oligonucleotide chain that can form a hairpin structure, with the stem of the hairpin comprising a hybridized double-stranded portion and a single-stranded portion, or the stem of the hairpin comprising a double-stranded portion while the single-stranded portion being the open-loop portion of the hairpin. In the case of hairpin structures, the oligonucleotide adapter can also be considered to be composed of an oligonucleotide chain that can form a hairpin structure. The oligonucleotide adapter comprises the second restriction nicking enzyme recognition sequence but lacks the cleavage site of the second restriction nicking enzyme, i.e., lacks a sequence that can be cleaved by the second restriction nicking enzyme, and only has its complementary sequence, which constitutes the single-stranded portion of said oligonucleotide adapter. The single-stranded portion of the oligonucleotide adapter can hybridize with the single-stranded region of the target double-strand, and the double-stranded structure formed after hybridization of the oligonucleotide adapter with the single-stranded region of the target double-strand can be recognized by the second restriction nicking enzyme and undergo cleavage at a predetermined position near this single-stranded region. Specifically, the hybridization of the oligonucleotide adapter with the single-stranded region of the target double-stranded stranded DNA brings nucleotides on the other strand of the target double-stranded DNA in close proximity to said recognition sequence and causes the predetermined position on that other strand to be located exactly where the cleavage site of said second restriction nicking enzyme is located, thereby enabling said second restriction nicking enzyme to cleave at the predetermined position on the other strand of said target double-stranded DNA by recognizing the recognition sequence on the oligonucleotide adapter.

The enzyme digestion of the target double-stranded DNA using an oligonucleotide adapter in conjunction with a second restriction nicking enzyme as described in the method provided in the second aspect of the present invention may also be referred to as assisted enzyme digestion.

When the single-stranded portion of the oligonucleotide adapter hybridizes with the single-stranded region of the target double-stranded DNA or with a predetermined region of the single-stranded DNA, the hybridized region extends from the first single-stranded nucleotide in the oligonucleotide adapter immediately adjacent to the double-stranded portion in a direction away from the double-stranded portion. The length of the double-stranded portion of the oligonucleotide adapter can be between 6 to 30 bases, preferably 10 to15 bases, and the length of its single-stranded portion can be 5 to 50 bases, preferably 10 to 30 bases, more preferably 15 to 20 bases. The length of the hybridization region of the single-stranded portion of the oligonucleotide adapter with the other strand of the target double-stranded DNA or the single-stranded DNA can be greater than, equal to, or less than the length of its single-stranded portion, for example, it can be 5 to 100 bases, preferably 10 to 80 bases, more preferably 15 to 70 bases.

In the assisted enzyme digestion method of the present invention, when the single-stranded region of the target double-stranded DNA is located at one end thereof and has a double-stranded region at the other end of the single-stranded region, for example, for the target double-stranded DNA obtained by the single-cleavage method and opposite direction double-cleavage method, the single-stranded portion of the oligonucleotide adapter and the single-stranded region of the target double-stranded DNA are preferably oriented such that, when the two hybridize, the nucleotides in the single-stranded portion of the oligonucleotide adapter near the double-stranded portion of the oligonucleotide adapter hybridize with the portion of the single-stranded region of the target double-stranded DNA near the double-stranded region of the target double-stranded DNA, and the nucleotides in the single-stranded portion of the oligonucleotide adapter away from the double-stranded portion of the oligonucleotide adapter hybridize with the portion of the single-stranded region of the target double-stranded DNA away from the double-stranded region of the target double-stranded DNA. The orientation of the single-stranded portion of the oligonucleotide adapter and the orientation of the single-stranded region of the target double-stranded DNA depend on the second restriction nicking enzyme used. Specifically, when the second restriction nicking enzyme is the first class restriction nicking enzyme (e.g., Nt.BstNBI, Nt.AlwI, Nt.BspQI, or Nt.BsmAI), the single-stranded portion of the oligonucleotide adapter is oriented from the nucleotide immediately adjacent to the double-stranded portion toward the nucleotide away from the double-stranded portion as 3′ to 5′, and the corresponding single-stranded region of the target double-stranded DNA is oriented from nucleotides in the immediately adjacent double-stranded region toward nucleotides away from the double-stranded region as 5′ to 3′. When the second restriction nicking enzyme is the second class restriction nicking enzyme (e.g., Nb.BtsI or Nb.BsrDI), the single-stranded portion of the oligonucleotide adapter is oriented from the nucleotide immediately adjacent to the double-stranded portion toward the nucleotide away from the double-stranded portion as 5′ to 3′, and the corresponding single-stranded region of the target double-stranded DNA is oriented from nucleotides immediately adjacent to the double-stranded region toward nucleotides away from the double-stranded region as 3′ to 5′.

When a target double-stranded DNA with a double-stranded region and a single-stranded region is generated by the same direction double-cleavage method, the target double-stranded DNA has a single-stranded region and two double-stranded regions located at each end of the single-stranded region, respectively. When such a single-stranded region of the target double-stranded DNA hybridizes with the single-stranded portion of the oligonucleotide adapter, one and only one of the double-stranded regions matches the following description: “the nucleotide in the single-stranded portion of the oligonucleotide adapter near the double-stranded portion hybridizes with the portion of the single-stranded region of the target double-stranded DNA near that double-stranded region, and the nucleotide in the single-stranded portion of the oligonucleotide adapter away from the double-stranded portion hybridizes with the portion of the single-stranded region of the target double-stranded DNA away from that double-stranded region.” The end immediately adjacent to the double-stranded region matching the description is the end desired to be manipulated in the next step, in other words, the final predetermined end generated is the end extending from the double-stranded region matching the description. In the following, for target double-stranded DNA generated by same direction double-cleavage method, the double-stranded region that matches this description can also be referred to as the double-stranded region of interest.

Which double-stranded region to use as the double-stranded region of interest depends on the second restriction nicking enzyme used. Specifically, when the second restriction nicking enzyme is the first class restriction nicking enzyme (e.g., Nt.BstNBI, Nt.AlwI, Nt.BspQI, or Nt.BsmAI), the double-stranded region of interest is the double-stranded region immediately adjacent to the 5′ side of the single-stranded region, or rather the double-stranded region immediately adjacent to the 3′ side of the dissociated DNA single strand. When the second restriction nicking enzyme is the second class restriction nicking enzyme (e.g., Nb.BtsI or Nb.BsrDI), the double-stranded region of interest is the double-stranded region immediately adjacent to the 3′ side of the single-stranded region, or rather the double-stranded region immediately adjacent to the 5′ side of the dissociated DNA single strand.

The structure of the oligonucleotide adapter is related to the second restriction nicking enzyme used. When the second restriction nicking enzyme is a first class restriction nicking enzyme (Nt.BstNBI, Nt.AlwI, Nt.BspQI, or Nt.BsmAI), on the oligonucleotide adapter, the recognition sequence of the second restriction nicking enzyme is located on the strand that does not have a single-stranded portion of the two strands of the double-stranded portion, and the recognition sequence is located entirely on the double-stranded portion, and 3′ side of this recognition sequence lacks a cleavage site, and the single-stranded portion of the oligonucleotide adapter is oriented from the nucleotide immediately adjacent to the double-stranded portion toward the nucleotide away from the double-stranded portion as 3′ to 5′. In this case, in some embodiments, when the recognition sequence of the second restriction nicking enzyme used is spaced by 1 or more nucleotides from its cleavage site (e.g. the second restriction nicking enzyme used is Nt.BstNBI, Nt.AlwI, Nt.B spQI or Nt.BsmAI), the second restriction nicking enzyme recognition sequence can be immediately adjacent to the end of the double-stranded portion on its cleavage site side in said oligonucleotide adapter, and the assisted enzyme digestion using such oligonucleotide adapters is referred to as the immediate adjacent mode in the present invention. In some embodiments, when the recognition sequence of the second restriction nicking enzyme used is spaced by 2 or more nucleotides from its cleavage site (e.g., the second restriction nicking enzyme used is Nt.BstNBI or Nt.AlwI), in the oligonucleotide adapter, there can be 1 nucleotide between the second restriction nicking enzyme recognition sequence and the end of the double-stranded portion on its cleavage site side, and the assisted enzyme digestion using such oligonucleotide adapters is referred to as the inter-1 mode in the present invention. In other embodiments, when the recognition sequence of the second restriction nicking enzyme used is spaced by 3 or more nucleotides from its cleavage site (e.g., the second restriction nicking enzyme used is Nt.BstNBI or Nt.AlwI), in the oligonucleotide adapter, there can be 2 nucleotides between the second restriction nicking enzyme recognition sequence and the end of the double-stranded portion on its cleavage site side, and the assisted enzyme digestion using such oligonucleotide adapters is referred to as the inter-2 mode in the present invention. In other embodiments, when the recognition sequence of the second restriction nicking enzyme used is spaced by 4 or more nucleotides from its cleavage site (e.g., the second restriction nicking enzyme used is Nt.BstNBI or Nt.AlwI), in the oligonucleotide adapter, there can be 3 nucleotides between the second restriction nicking enzyme recognition sequence and the end of the double-stranded portion on its cleavage site side, and the assisted enzyme digestion using such oligonucleotide adapters is referred to as the inter-3 mode in the present invention. In the above case, the number of nucleotides between the second restriction nicking enzyme recognition sequence and the ends of the double-stranded portion on its cleavage site side should be less than the number of characteristic nucleotides of the restriction nicking enzyme used. Thus, which mode can be used depends on the number of characteristic nucleotides of the second restriction nicking enzyme, and the number of nucleotides between the second restriction nicking enzyme recognition sequence and the ends of the double-stranded portion on its cleavage site side in the oligonucleotide adapter should be less than the number of characteristic nucleotides of said second restriction nicking enzyme. If the number of characteristic nucleotides of the second restriction nicking enzyme is 1, then the immediate neighbor mode is used, and if the number of characteristic nucleotides of the second restriction nicking enzyme is 4, then the immediate neighbor mode, the inter-1 mode, the inter-2 mode or the inter-3 mode can be used. In other embodiments, the oligonucleotide adapter may also have more than 3 nucleotides between the second restriction nicking enzyme recognition sequence and the end of the double-stranded portion on its cleavage site side if the number of characteristic nucleotides of the second restriction nicking enzyme used is greater than 4.

In some embodiments, when the second restriction nicking enzyme used is Nt.BstNBI, the 3′ end of the strand of the two strands of the double-stranded portion of the oligonucleotide adapter that does not have a single-stranded portion may be —GAGTC, —GAGTCN, —GAGTCNNN, or —GAGTCNNN. When the second restriction nicking enzyme used is Nt.AlwI, the 3′ end of the two strands of the double-stranded portion of the oligonucleotide adapter that does not have a single-stranded portion may be —GGATC, GGATCN, —GGATCNN, or —GGATCNNN. When the second restriction nicking enzyme used is Nt.BspQI, the 3′ end of the strand that does not have the single-stranded portion of the two strands of the double-stranded portion of the oligonucleotide adapter may be -GCTCTTC. When the second restriction nicking enzyme used is Nt.BsmAI, the 3′ end of the strand that does not have the single-stranded portion of the two strands of the double-stranded portion of the oligonucleotide adapter may be —GTCTC. Where “−” indicates that other nucleotides can also be covalently attached and N indicates that it can be any nucleotide, such as A, T, C or G.

When the second class of restriction nicking enzyme is chosen (e.g., Nb.BtsI or Nb.BsrDI) as the second restriction nicking enzyme, on the oligonucleotide adapter, the recognition sequence of the second restriction nicking enzyme is located on the strand having a single-stranded portion of the two strands of the double-stranded portion, the 5′ side of the complementary hybridization sequence of this recognition sequence lacks the cleavage site, and the single-stranded portion of the oligonucleotide adapter is oriented from the nucleotide immediately adjacent to the nucleotide of the double-stranded portion toward the nucleotide away from the double-stranded portion as 5′ to 3′. The presently known second class restriction nicking enzymes have a cleavage site immediately adjacent to the 5′ end of the complementary sequence of its recognition sequence on the minus strand, so that if cleavage is to be achieved, at least the last nucleotide at 5′ end of the complementary sequence on the minus strand needs to be provided by the single strand of the target double-stranded DNA to provide the complete cleavage site. In this case, the recognition sequence of the second restriction nicking enzyme in the oligonucleotide adapter is not located entirely on its double-stranded portion, but rather one nucleotide at 3′ end of the recognition sequence is located on the single-stranded portion and the other nucleotides of the recognition sequence are located on the double-stranded portion, and accordingly, at least the last nucleotide at 5′ end of the complementary sequence of the recognition sequence on the minus strand is not included in the oligonucleotide adapter. At least the last nucleotide of the 5′ end obtained by cleavage using such a second restriction nicking enzyme in conjuction with the oligonucleotide adapter cannot be tailored and is necessarily identical to the last nucleotide at 5′ end of the complementary sequence of the recognition sequence on the minus strand.

In some embodiments, when the second restriction nicking enzyme used is Nb.BsrDI, the 5′ end of the strand of the two strands of the double-stranded portion of the oligonucleotide adapter that does not have a single-stranded portion may be ATTGC-, while on the strand of the two strands of the double-stranded portion of the oligonucleotide adapter that has a single-stranded portion, the sequence located on 5′ side of the demarcation point between the double-stranded portion and the single-stranded portion is —GCAAT, and the sequence located on 3′ side of the demarcation point between the double-stranded portion and the single-stranded portion is G-. When the second restriction nicking enzyme used is Nb.BtsI, the 5′ end of the strand of the double-stranded portion of the oligonucleotide adapter that does not have a single-stranded portion may be ACTGC-, while on the strand of the two strands of the double-stranded portion of the oligonucleotide adapter that has a single-stranded portion, the sequence located on 5′ side of the demarcation point between the double-stranded portion and the single-stranded portion is —GCAGT, and the sequence located on 3′ side of the demarcation point between the double-stranded portion and the single-stranded portion is G-. When Nb.BsrDI or Nb.BtsI is utilized as the second restriction nicking enzyme, at least the last nucleotide of the 5′ end obtained by its cleavage in conjuction with the oligonucleotide adapter is necessarily C.

The variety of restriction nicking enzymes continues to increase, and in the future, if a restriction nicking enzyme occurs where the cleavage position is not on the same strand as the recognition sequence and there are non-customized bases between the cleavage site and the complementary sequence of the recognition sequence on the minus strand, such as the complementary sequence of the recognition sequence on the minus strand itself being separated from the cleavage position by 1, 2, 3, 4, 5 or more nucleotides, such a restriction nicking enzyme can also be used as the second restriction nicking enzyme to obtain a fully customized end. For such a restriction nicking enzyme, the number of nucleotides between the complementary sequence of the recognition sequence on the minus strand and the cleavage position is called the number of characteristic nucleotides. In this case, on the oligonucleotide adapter, the recognition sequence of the second restriction nicking enzyme is located on the strand of the two strands of the double-stranded portion that has a single-stranded portion, and the recognition sequence is located entirely on the double-stranded portion, the cleavage site is missing on 5′ side of the complementary sequence of this recognition sequence on the minus strand, and the single-stranded portion of the oligonucleotide adapter is oriented from the nucleotide immediately adjacent to the double-stranded portion to the nucleotide away from the double-stranded portion as 5′ to 3′. In this case, in some embodiments, when the complementary sequence of the recognition sequence of the second restriction nicking enzyme used on the minus strand is spaced 1 or more nucleotides from its cleavage site, the complementary sequence of the second restriction nicking enzyme recognition sequence on the minus strand may be immediately adjacent to the end of the double-stranded portion on its cleavage site side in said oligonucleotide adapter, or have a spacing of 1, 2, 3 or more nucleotides. Similarly, if such a restriction nicking enzyme is used as the second restriction nicking enzyme, the number of nucleotides between the complementary sequence of the second restriction nicking enzyme recognition sequence on the minus strand and the end of the double-stranded portion on its cleavage site side in the oligonucleotide adapter should be less than the number of characteristic nucleotides of said second restriction nicking enzyme.

Regarding Cleavage to Generate the Predetermined End

The oligonucleotide adapter can be designed such that the second restriction nicking enzyme cleaves the other strand of the target double-stranded DNA or single-stranded DNA at a predetermined position.

When the first class restriction nicking enzyme is chosen as the second restriction nicking enzyme (e.g., Nt.BstNBI, Nt.AlwI, Nt.BspQI, or Nt.BsmAI), the position on the other strand of the target double-stranded DNA to be cleaved is determined by a combination of factors, one of which is the number of characteristic nucleotides of the particular second restriction nicking enzyme used, another factor is the number of nucleotides between the recognition sequence and the end of the double-stranded portion on the cleavage site side in the oligonucleotide adapter, and yet another factor is the position of the hybridization start base of the other strand of the target double-stranded DNA in that other strand. Said hybridization start base is the nucleotide in the hybridization region of the other strand of the target double-stranded DNA (i.e., the sequence that hybridizes to the single-stranded portion of the oligonucleotide adapter) that is immediately adjacent to the double-stranded portion of the oligonucleotide adapter. The position cleaved by the second restriction nicking enzyme is, along the direction from 5′ to 3′, after a specific number of nucleotides from the hybridization start base of the target double-stranded DNA, wherein the specific number is the number of characteristic nucleotides of the particular second restriction nicking enzyme used minus the number of bases between the recognition sequence and the end of the double-stranded portion on its cleavage site side in the oligonucleotide adapter. When cleaving the target single-stranded DNA, the factors that determine where on the target single-stranded DNA is cleaved include (1) the number of characteristic nucleotides of the particular second restriction nicking enzyme used, (2) the number of nucleotides between the recognition sequence and the end of the double-stranded portion on the cleavage site side in the oligonucleotide adapter, and (3) the position of the hybridization start base of the target single-stranded DNA in that single-stranded DNA. The hybridization start base of the target single-stranded DNA referred to herein is the nucleotide in the hybridization region of the target single-stranded DNA (i.e., the sequence that hybridizes to the single-stranded portion of the oligonucleotide adapter) that is immediately adjacent to the double-stranded portion of the oligonucleotide adapter. The position cleaved by the second restriction nicking enzyme is, along the direction from 5′ to 3′, after a specific number of nucleotides from the hybridization start base of the target single-stranded DNA, wherein the specific number is the number of characteristic nucleotides of the particular second restriction nicking enzyme used minus the number of bases between the recognition sequence and the end of the double-stranded portion on its cleavage site side in the oligonucleotide adapter.

When the second class restriction nicking enzyme, such as Nb.BsrDI or Nb.BtsI, is chosen as the second restriction nicking enzyme, since the partial sequence at the junction of the single-portion and double-stranded portion of the corresponding oligonucleotide adapter has been determined, the factor that determine the position of the cleaved site on the target single-stranded DNA includes only the position of the hybridization start base of the target single-stranded DNA in that single-stranded DNA. The position cleaved by the second restriction nicking enzyme is, along the direction from 3′ to 5′, after one nucleotide from the hybridization start base of the target single-stranded DNA.

With the assisted enzyme digestion mode, cleavage can be made at different predetermined positions on the other strand of the target double-stranded DNA to generate the desired end, and the cleavage position can be located either on the single-stranded region of the target double-stranded DNA or on the double-stranded region of the target double-stranded DNA.

(1) Assisted Double-Stranded Enzyme Digestion

In some embodiments, the hybridization region of the other strand of the target double-stranded DNA (i.e., the region on the target double-stranded DNA that hybridizes to the single-stranded portion of the oligonucleotide adapter) is located on the single-stranded region and immediately adjacent to its double-stranded region, or is separated from the double-stranded region by one or more nucleotides, such as one, two or three or more nucleotides. In this case, when different enzymes are chosen as the second restriction nicking enzyme, different cleavage results are obtained.

If the second restriction nicking enzyme used is the first class restriction nicking enzyme, such as, Nt.BstNBI, Nt.AlwI, Nt.BspQI, or Nt.BsmAI, then the assisted enzyme digestion using oligonucleotide adapters can result in 3′ overhangs of different lengths, with the number of overhanging bases being the number of characteristic nucleotides of the second restriction nicking enzyme used minus the number of nucleotides between the recognition sequence and the end on its cleavage site side in the oligonucleotide adapter used, then plus the number of nucleotides between the hybridization region and the double-stranded region of the target double-stranded DNA. In this case, the number of nucleotides between the recognition sequence and the end of its cleavage site side in the oligonucleotide adapter should be less than the number of characteristic nucleotides of the second restriction nicking enzyme used.

When Nt.BstNBI or Nt.AlwI is used as the second restriction nicking enzyme, both of them have a number of characteristic nucleotides of 4. If the number of nucleotides between the recognition sequence and 3′ end of the strand in which it is located in the oligonucleotide adapter is m, and the number of nucleotides separating the hybridization region of the single-stranded region from the double-stranded region of the target double-stranded DNA is n, the number of bases in the resulting 3′ overhang is 4-m+n, provided that m is less than 4. For example, when m=2, a 3′ overhang of 2 bases is generated if the number n of nucleotides separating the hybridization region of the single-stranded region from the double-stranded region of the target double-stranded DNA is 0, and a 3′ overhang of 3 bases is generated if the number n of nucleotides separating the hybridization region of the single-stranded region from the double-stranded region of the target double-stranded DNA is 1; a 3′ overhang of 4 bases is generated if the number n of nucleotides separating the hybridization region of the single-stranded region from the double-stranded region of the target double-stranded DNA is 2; a 3′ overhang of 5 bases or more is generated if the number n of nucleotides separating the hybridization region of the single-stranded region from the double-stranded region of the target double-stranded DNA is 3 or more. However, in this case, if the first restriction nicking enzyme is identical to the second restriction nicking enzyme, the sequence of the 3′ overhang starting from the 5^{th} nucleotide from 5′ to 3′ direction comprises the nucleotide of the recognition sequence of said first restriction nicking enzyme, which is determined by the way the first restriction nicking enzyme cleaves the target double-stranded DNA. Further, for example, when m=0, a 3′ overhang of 4 bases is generated if the number n of nucleotides separating the hybridization region of the single-stranded region from the double-stranded region of the target double-stranded DNA is 0, and a 3′ overhang of 5 or more bases is generated if the number n of nucleotides separating the hybridization region of the single-stranded region from the double-stranded region of the target double-stranded DNA is 1 or more, similarly, in this case, if the first restriction nicking enzyme is identical to the second restriction nicking enzyme, the sequence of the 3′ overhang starting from the 5^{th} base from 5′ to 3′ direction comprises the recognition sequence for said first restriction nicking enzyme. Further, for example, when m=1, a 3′ overhang of 3 bases is generated if the number n of nucleotides separating the hybridization region of the single-stranded region from the double-stranded region of the target double-stranded DNA is 0, a 3′ overhang of 4 bases is generated if the number n of nucleotides separating the hybridization region of the single-stranded region from the double-stranded region of the target double-stranded DNA is 1, and a 3′ overhang of 5 bases or more is generated if the number n of nucleotides separating the hybridization region of the single-stranded region from the double-stranded region of the target double-stranded DNA is 2 or more, similarly, in this case, if the first restriction nicking enzyme is identical to the second restriction nicking enzyme, the sequence of the 3′ overhang starting from the 5^{th} base from 5′ to 3′ direction comprises the recognition sequence of said first restriction nicking enzyme. Further, for example, when m=3, a 3′ overhang of 1 base is generated if the number n of nucleotides separating the hybridization region of the single-stranded region from the double-stranded region of the target double-stranded DNA is 0; a 3′ overhang of 2 bases is generated if the number n of nucleotides separating the hybridization region of the single-stranded region from the double-stranded region of the target double-stranded DNA is 1; and a 3′ overhang of 3 bases is generated if the number n of nucleotides separating the hybridization region of the single-stranded region from the double-stranded region of the target double-stranded DNA is 2. a 3′ overhang of 4 bases is generated if the number n of nucleotides separating the hybridization region of the single-stranded region from the double-stranded region of the target double-stranded DNA is 3; a 3′ overhang of 5 bases or more is generated, if the number n of nucleotides separating the hybridization region of the single-stranded region from the double-stranded region of the target double-stranded DNA is 4 or more, similarly, in this case, the sequence of the 3′ overhang starting from the 5^{th} base of 5′ to 3′ direction comprises the recognition sequence for said restriction nicking enzyme. In this paragraph, when referring to the double-stranded region of the target double-stranded DNA, for the target double-stranded DNA obtained by the same direction double-cleavage method, it refers to its double-stranded region of interest, which is defined as described previously.

When Nt.B spQI or Nt.B smAI is used as the second restriction nicking enzyme, both of them have a number of characteristic nucleotides of 1. In the oligonucleotide adapter, the number m of nucleotides between the recognition sequence and 3′ end of the strand in which it is located is 0. At this time, the assisted enzyme digestion using the oligonucleotide adapter can result in 3′ overhangs of different lengths, with the number of overhanging bases being the number n of nucleotides between the hybridization region and the double-stranded region of the target double-stranded DNA plus 1 (n+1). A 3′ overhang of 1 base is generated if the number n of nucleotides separating the hybridization region of the single-stranded region from the double-stranded region of the target double-stranded DNA is 0; a 3′ overhang of 2 bases is generated if the number n of nucleotides separating the hybridization region of the single-stranded region from the double-stranded region of the target double-stranded DNA is 1; and a 3′ overhang of 3 bases or more is generated if the number n of nucleotides separating the hybridization region of the single-stranded region from the double-stranded region of the target double-stranded DNA is 2 or more. In this paragraph, when referring to the double-stranded region of the target double-stranded DNA, for the target double-stranded DNA obtained by the same direction double-cleavage method, it refers to its double-stranded region of interest, which is defined as described previously.

In some embodiments, where m=2 and n=2 (e.g. when the number of characteristic nucleotides of the second restriction nicking enzyme used is 3 or more, such as Nt.AlwI or Nt.BstNBI), there are only 2 nucleotides in the oligonucleotide adapter that need to hybridize with the nucleotides in the final resulting 3′ overhang, i.e., the 2 nucleotides starting from the first single-stranded nucleotide of the oligonucleotide adapter immediately adjacent double-stranded portion. Therefore, except for these oligonucleotides, all other nucleotides on the oligonucleotide adapter can be artificially selected as long as they can fulfill the function of the oligonucleotide adapter. For example, the double-stranded portion of the oligonucleotide adapter is feasible as long as it includes the second restriction nicking enzyme recognition site and the other sequences are not limited, while in the single-stranded portion of the oligonucleotide adapter, nucleotides other than the said two described need to be able to hybridize with the corresponding portion of the single-stranded region of the target double-stranded DNA, while this part of the single-stranded region of the target double-stranded DNA can be artificially added to the double-stranded DNA to be modified, and thus its sequence can also be not limited (except for the complementary sequence of the first restriction nicking enzyme recognition sequence contained therein). Thus, with the same second restriction nicking enzymes used, the nucleotides in the oligonucleotide adapter other than the said two nucleotides can be fixed (which means that the sequence of the double-stranded DNA fragment containing the first restriction nicking enzyme recognition sequence and added to the target double-stranded DNA is also fixed), and 16 oligonucleotide adapters are prepared according to all permutations of the the said two nucleotides, wherein each oligonucleotide adapter contains one permutation of the the said two nucleotides and 16 oligonucleotide adapters contain all 16 permutations of the the said two nucleotides. A mixture of these oligonucleotide adapters can be used as universal oligonucleotide adapters suitable for assisted enzyme digestion of a variety of different target double-stranded DNAs with n=2.

If the second restriction nicking enzyme used is the second class restriction nicking enzyme, such as Nb.BsrDI or Nb.BtsI, then the assisted enzyme digestion using the oligonucleotide adapter can result in 5′ overhangs of different lengths, with the number of overhanging bases being the number n of nucleotides between the hybridization region and the double-stranded region of the target double-stranded DNA plus 1 (n+1). A 5′ overhang of 1 base is generated if the number n of nucleotides separating the hybridization region of the single-stranded region from the double-stranded region of the target double-stranded DNA is 0; a 5′ overhang of 2 bases is generated if the number n of nucleotides separating the hybridization region of the single-stranded region from the double-stranded region of the target double-stranded DNA is 1; a 5′ overhang of 3 bases or more is generated if the number n of nucleotides separating the hybridization region of the single-stranded region from the double-stranded region of the target double-stranded DNA is 2 or more. In this paragraph, when referring to the double-stranded region of the target double-stranded DNA, for the target double-stranded DNA obtained by the same direction double-cleavage method, it refers to its double-stranded region of interest, which is defined as described previously.

(2) Invasive Assisted Double-Stranded Enzyme Digestion

In some other particular embodiments, the region on the target double-stranded DNA that hybridizes to the single-stranded portion of the oligonucleotide adapter includes not only the single-stranded region of that target double-stranded DNA, but also a portion of the double-stranded region sequence adjacent to that single-stranded region, in other words, the hybridization start base of the target double-stranded DNA is located on the double-stranded portion of the target double-stranded DNA. In this case, the single-stranded portion of the oligonucleotide adapter includes not only a sequence capable of hybridizing with the single-stranded region of the target double-stranded DNA, but also included between this sequence and the double-stranded portion of the oligonucleotide adapter a sequence capable of hybridizing to a segment in the double-stranded region of the target double-stranded DNA adjacent to the single-stranded region. This type of assisted enzyme digestion is referred to in the present invention as invasive assisted enzyme digestion, and the sequence in the single-stranded portion of the oligonucleotide adapter that can hybridize with a segment in the double-stranded region of the target double-stranded DNA adjacent to the single-stranded region is referred to as the “invading region”, the segment of the double-stranded region adjacent to the single-stranded region is called the “invaded region”. The invaded region originally hybridizes with its complementary sequence to form a double strand, and during the hybridization of the oligonucleotide adapter with the target double-stranded DNA, the sequence of the invaded region may undergo double-stranded dissociation and hybridize with the invading region. The invading region is immediately adjacent to the double-stranded portion of the oligonucleotide adapter at one end and to the sequence in the single-stranded portion of the oligonucleotide adapter that can hybridize with the single-stranded region of the target double-stranded DNA at one end. The length of the invading and invaded regions can be between 1 to 100 bases, preferably between 1 to 30 bases, and more preferably between 3 to 20 bases. In this paragraph, when referring to the double-stranded region of the target double-stranded DNA, for the target double-stranded DNA obtained by the same direction double-cleavage method, it refers to its double-stranded region of interest, which is defined as described previously.

If the second restriction nicking enzyme used is the first class restriction nicking enzyme, such as Nt.BstNBI, Nt.AlwI, Nt.BspQI or Nt.BsmAI, Invasive assisted enzyme digestion using oligonucleotide adapters can result in 3′ overhangs of different lengths, blunt ends, or 5′ overhangs of different lengths. A 3′ overhang is generated when the number of characteristic nucleotides of the second restriction nicking enzyme used minus the number of nucleotides separating the recognition sequence from the end on its cleavage site side in the oligonucleotide adapter used is greater than the number of nucleotides in the invading region, with the length of the overhang being the number of characteristic nucleotides of the second restriction nicking enzyme used minus the number of nucleotides separating the recognition sequence from the ends on its cleavage site side in the oligonucleotide adapter used, then minus the number of nucleotides in the invading region. A blunt end is generated when the number of characteristic nucleotides of the second restriction nicking enzyme used minus the number of nucleotides separating the recognition sequence from the end on its cleavage site side in the oligonucleotide adapter used equals the number of nucleotides in the invading region. A 5′ overhang is generated when the number of characteristic nucleotides of the second restriction nicking enzyme used, minus the number of nucleotides separating the recognition sequence from the ends on its cleavage site side in the oligonucleotide adapter used is less than the number of nucleotides in the invading region; The length of the overhang being the difference obtained by the number of nucleotides in the invading region minus a number obtained by subtracting the number of nucleotides spaced between the recognition sequence and the end of its cleavage site side in the oligonucleotide adapter used from the number of characteristic nucleotides of the restriction nicking enzyme used. In these cases, the number of characteristic nucleotides of the second restriction nicking enzyme used should be greater than the number of nucleotides spaced between the recognition sequence and the end of its cleavage site side in the oligonucleotide adapter used.

In some particular embodiments, the second restriction nicking enzymes used is Nt.AlwI or Nt.BstNBI, both of which have a number of characteristic nucleotides of 4 and whose cleavage sites are on 3′ side of the recognition sequence. If in the oligonucleotide adapter, the number of nucleotides between the recognition sequence and 3′ end of the strand in which it is located is m and the number of nucleotides in the invading region is i, then when 4-m >i, a 3′ overhang is generated and the length of the 3′ overhang is 4-m-i; when 4-m=i, a blunt end is generated; and when 4-m <i, a 5′ overhang is generated and the length of the 5′ overhang is i-(4-m). For example, in the case of m=2, a 3′ overhang of 2 bases is generated if i=0; a 3′ overhang of 1 base is generated if i=1; a blunt end is generated if i=2; and a 5′ overhang of length i−2 is generated if i >2.

In some particular embodiments, the second restriction nicking enzyme used is Nt.BspQI or Nt.BsmAI, both of which have a number of characteristic nucleotides of 1 and whose cleavage site is on 3′ side of the recognition sequence. In the oligonucleotide adapter, the number m of nucleotides between the recognition sequence and 3′ end of the strand in which it is located is 0, and the number of nucleotides in the invading region is i. Then, when i is 0, a 3′ overhang is generated, with a length of 1; when i=1, a blunt end is generated; when i>1, a 5′ overhang is generated, with a length of i−1.

If the second restriction nicking enzyme used is the second class restriction nicking enzyme, such as Nb.BsrDI or Nb.BtsI, then, if the number of nucleotides in the invading region is i is 0, a 5′ overhang is generated, with a length of 1; when i=1, a blunt end is generated; when i>1, a 3′ overhang is generated, with a length of i−1.

Regarding Methylation of Target Double-Stranded DNA

When the first restriction nicking enzyme and the second restriction nicking enzyme are different, the target double-stranded DNA can further be methylated using a methylase of the second restriction nicking enzyme so that the target double-stranded DNA cannot be cleaved by the second restriction nicking enzyme.

the methylase of the nicking enzyme as used herein is a methylase whose recognition sequence is identical to or comprises the recognition sequence of the nicking enzyme. When the target double-stranded DNA is methylated by the methylase of the second restriction nicking enzyme, the target double-stranded DNA will not be cleaved by the corresponding second restriction nicking enzyme, but the subsequent cleavage of the other strand is not affected by the methylation of the target double-stranded DNA, because the cleavage of the other strand is mediated by the oligonucleotide adapter, which is not methylated.

For example, if the second restriction nicking enzyme is Nt.BstNBI, its corresponding methylase is M.BstNBI (or may be called bstNBIM), the recognition sequence of this methylase is GASTC, where S can be G or C, thus the GASTC sequence comprises GAGTC, then when the target double-stranded DNA is methylated by M.BstNBI, it will no longer be cleaved by the second restriction nicking enzyme Nt.BstNBI.

As known to those skilled in the art, restriction and modification enzymes (i.e., methylases) belong to the bacterial restriction-modification system (R-M), in which restriction enzymes are used to degrade exogenous DNA, thereby preventing its replication and integration into the host cell, and modification enzymes methylate the bacteria's own bases, thereby protecting their own DNA from degradation. For restriction enzymes, there usually are their corresponding methylases whose recognition sequences are identical to or comprises the recognition sequences of the corresponding restriction enzymes. Restriction nicking enzymes belong to restriction enzymes that also have corresponding methylases whose recognition sequence is identical to or comprises the recognition sequence of the corresponding nicking enzyme.

Methylases for different restriction nicking enzymes are well known to those skilled in the art, for example M.BstNBI recognizes GASTC and methylates N4-cytosine or N6-adenine; M.MlyI recognizes GASTC and methylates N6-adenine; both M.BstNBI and M.MlyI are suitable as methylases of Nt.BstNBI. For example, M.AlwI recognizes GGATC and methylates N6-adenine; M.AlwI is a methylase of Nt.AlwI. Then again, for example, M.BsmAI recognizes GTCTC and methylates cytosine, and M.BsmAI is a methylating enzyme of Nt.BsmAI. Information of these methylases is available from http://rebase.neb.com/rebase/index.html, from which other methylases of the second restriction nicking enzyme used in the present invention can also be obtained.

Methylation of target double-stranded DNA can be performed either in vitro or in vivo. In vitro, the target double-stranded DNA can be methylated by an isolated methylase; while in vivo, both the target double-stranded DNA and the expression gene of methylase can present together in the host cell, which expresses the methylase, at this time, if the recognition sequence of this methylase is present in the target double-stranded DNA, it will be methylated, and when the target double-stranded DNA is extracted, it is itself already in a methylated state. The gene encoding the methylase may be present on the same DNA double-strand (e.g., plasmid) as the target double-stranded DNA, or it may be present on other DNA molecules (e.g., present on another plasmid in the same host, or integrated into the host's genome) to enable expression of the methylase in said host.

In some particular embodiments, the second restriction nicking enzyme used to cleave the other strand is Nt.BstNBI or Nt.AlwI, and the target double-stranded DNA is co-existing with the M.BstNBI gene or the M.AlwI gene in the same host. DNA obtained from this host is naturally characterized to be methylated at the Nt.BstNBI or Nt.AlwI sites, and when target double-stranded DNA obtained from this host is used for assisted enzyme digestion, the target double-stranded DNA will not be cleaved by Nt.BstNBI or Nt.AlwI, but will be cleaved by other nicking enzymes such as Nt.BspQI, Nb.BbvCI or Nt.BbvCI. In the presence of other restriction nicking enzymes, a single-stranded region is generated on the target double-stranded DNA, and subsequent cleavage of the other strand is unaffected by methylation of the target double-stranded DNA because cleavage of the other strand is mediated by an oligonucleotide adapter, which is not methylated.

As previously mentioned, the method of the present invention can also be applied to cleave single-stranded DNA. Accordingly, a third aspect of the present invention provides a method of generating a cleavage at any predetermined position on a target single-stranded DNA, which comprises:

hybridizing a predetermined region of a target single-stranded DNA with a single-stranded portion of an oligonucleotide adapter, said oligonucleotide adapter is a DNA molecule having a double-stranded portion and a single-stranded portion, wherein the oligonucleotide adapter contains in the double-stranded portion a recognition site for a restriction nicking enzyme but lacks a sequence cleavable by the restriction nicking enzyme, wherein the single-stranded portion of the oligonucleotide adapter is capable of hybridizing with a single-stranded region of the target double-stranded DNA to form a double-stranded structure that is recognizable by the restriction nicking enzyme, and thereby leaves the predetermined position on the target double-stranded DNA in a position that can be cleaved by said restriction nicking enzyme by recognition of the recognition site on the oligonucleotide adapter by said restriction nicking enzyme;

generating a cleavage at the predetermined position on said target single strand using said restriction nicking enzyme.

It will be understood by those skilled in the art that in the above method of generating a cleavage at any predetermined position on the target single-stranded DNA, by “a double-stranded structure that is recognizable by said restriction nicking enzyme” is meant that when the single-stranded portion of said oligonucleotide adapter hybridizes with the single-stranded region of the target double-stranded DNA, the hybridized region and the double-stranded portion of said oligonucleotide adapter together form a double-stranded structure recognizable and cleavable by said restriction nicking enzyme, said restriction nicking enzyme recognizes the recognition site on the double-stranded portion of said oligonucleotide adapter and cleaves the other strand of the target double-stranded DNA at t predetermined position. Those skilled in the art can appreciate that a predetermined region of the target single-stranded DNA hybridizes with the single-stranded portion of the oligonucleotide adapter to form a hybridized region immediately adjacent to the double-stranded portion of the oligonucleotide adapter, whereby said restriction nicking enzyme recognizes a recognition site on said double-stranded portion of the oligonucleotide adapter and cleaves the predetermined position on the target single-stranded DNA.

This enzyme digestion against the target single-stranded DNA is equivalent to a customized single-stranded DNA restriction endonuclease. Said predetermined position refers to an artificially selected position on the target single-stranded DNA to be cleaved, which can be between any two adjacent bases on the target single-stranded DNA.

The types and structures of restriction nicking enzymes and oligonucleotide adapters used in the cleavage of the target single-stranded DNA are the same as in the method of the first aspect described previously.

There are no limitations on the sequence of the target single-stranded DNA, nor on its source, including but not limited to: an oligonucleotide synthesized by a DNA synthesizer, single-stranded DNA obtained from a plasmid with an f1 replicon under a phagemid rescue operation, single-stranded DNA generated in a rolling loop replication, single-stranded DNA obtained from double-stranded DNA under denaturing conditions, and the like.

The temperature of the cleavage process in the assisted digestion of the present invention may be fixed (referred to as isothermal assisted enzyme digestion) or may be variable (referred to as non-isothermal assisted enzyme digestion). The temperature of isothermal assisted enzyme digestion may be between 37 to 75 degrees Celsius, preferably 45 to 65 degrees Celsius, more preferably 55 degrees Celsius.

Non-isothermal assisted enzyme digestion means that different temperatures are used for different steps in the cleavage process, and the digestion is carried out in a temperature cycling manner. For example, a higher temperature may be used when generating one or more nicks at a predetermined position on one strand of the target double-stranded DNA, and a lower temperature may be used when using oligonucleotide adapters in combination with the use of the corresponding restriction nicking enzyme to generate a cleavage at a predetermined position on the other strand of the target double-stranded DNA. Thus, in isothermal assisted enzyme digestion, temperature cycling can be performed throughout the reaction, with the highest temperature of the cycle between 50 to 75 degrees Celsius, preferably 55 to 65 degrees Celsius, and the lowest temperature of the cycle between 37 to 55 degrees Celsius, preferably 45 to 55 degrees Celsius, with each cycle lasting between 30 seconds to 20 minutes, preferably 1 minute to 5 minutes.

In the whole reaction system of the present invention, D-trehalose can also be added to improve the rate of enzyme digestion, and the concentration of D-trehalose is 0.1M to 2M, preferably 0.2M to 0.6M.

In the present invention, the terms “base” and “nucleotide” are used interchangeably and both refer to the individual deoxyribonucleotides that constitutes double-stranded DNA or single-stranded DNA.

If not specifically indicated, the sequences mentioned in the present invention are all oriented from 5′ to 3′.

### BRIEF DESCRIPTION OF THE DRAWINGS

The present invention will be more fully understood by the following detailed description and in conjunction with the accompanying drawings, wherein:

FIG. 1 shows enzyme digestion mode of FokI, where the arrow points to the cleavage position.

FIG. 2 shows enzyme digestion mode of Nt.BstNBI, where “. . . ” indicates a DNA sequence of variable length, and this symbol fits all the figures of the present invention.

FIG. 3 shows three assisted single-stranded enzyme digestion modes.

FIG. 4 shows a method of generating single-stranded region.

FIG. 5 shows the composition of the oligonucleotide adapter composition.

FIG. 6 shows assisted double-stranded enzyme digestion to generate a 3′ overhang of 4 bases.

FIG. 7 shows assisted double-stranded enzyme digestion to generate a 3′ overhang of length greater than or equal to 2 bases.

FIG. 8 shows invasive assisted double-stranded enzyme digestion.

FIG. 9 shows assisted single-stranded enzyme digestion with restriction nicking enzyme Nt.BstNBI.

FIG. 10 shows an electropherogram of assisted single-stranded enzyme digestion using restriction nicking enzyme Nt.BstNBI.

FIG. 11 shows a process of generating a 3′ overhang of 4 bases by assisted double-stranded enzyme digestion using restriction nicking enzyme Nt.BstNBI.

FIG. 12 shows an electropherogram of a 3′ overhang of 4 bases generated assisted double-stranded enzyme digestion using restriction nicking enzyme Nt.BstNBI.

FIG. 13 shows generation of a 3′ overhang of 4 bases for DNA splicing.

FIG. 14 is an example of invasive assisted double-stranded enzyme digestion to generate overhangs of different lengths.

FIG. 15 is an electropherogram of invasive-assisted double-stranded enzyme digestion to generate blunt ends and 5′ hanging ends.

FIG. 16 is an example of Nt+Nt enzyme digestion pattern, where the first restriction nicking enzyme is a restriction nicking enzyme of the Nt series and the second restriction nicking enzyme is a restriction deletion nicking enzyme of the Nt series.

FIG. 17 is an example of Nb+Nb enzyme digestion pattern, where the first restriction nicking enzyme is a restriction nicking enzyme of the Nb series and the second restriction nicking enzyme is a restriction nicking enzyme of the Nb series.

FIG. 18 is an example of Nb+Nt digestion pattern, where the first restriction nicking enzyme is a restriction nicking enzyme of the Nb series and the second restriction nicking enzyme is a restriction nicking enzyme of the Nt series.

FIG. 19 is an example of Nt+Nb digestion pattern, wherein the first restriction nicking enzyme is a restriction nicking enzyme of the Nt series and the second restriction nicking enzyme is a restriction nicking enzyme of the Nb series.

FIG. 20 shows an electropherogram of two nicking enzymes +a second restriction nicking enzyme methylase used for assisted enzyme digestion.

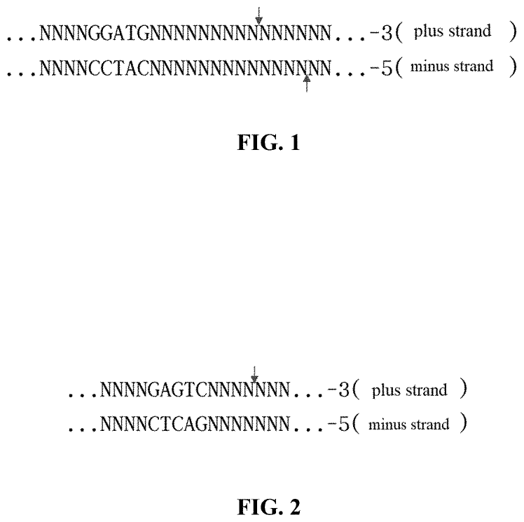

### DETAILED DESCRIPTION OF THE INVENTION

The present invention relates to a method of manipulating ends of double-stranded DNA by using a restriction nicking enzyme to first generate one or more nicks on the double-strand to generate a single-stranded region, and then using hybridization of an oligonucleotide adapter with the single-stranded region in combination with the same restriction nicking enzyme to generate a cleavage on the other strand of the DNA double-strand. The position of this cleavage correlates with the sequence selection for the oligonucleotide adapter, which ultimately cleaves the target double-stranded DNA and generates ends of controlled length and hanging species at the cleavage.

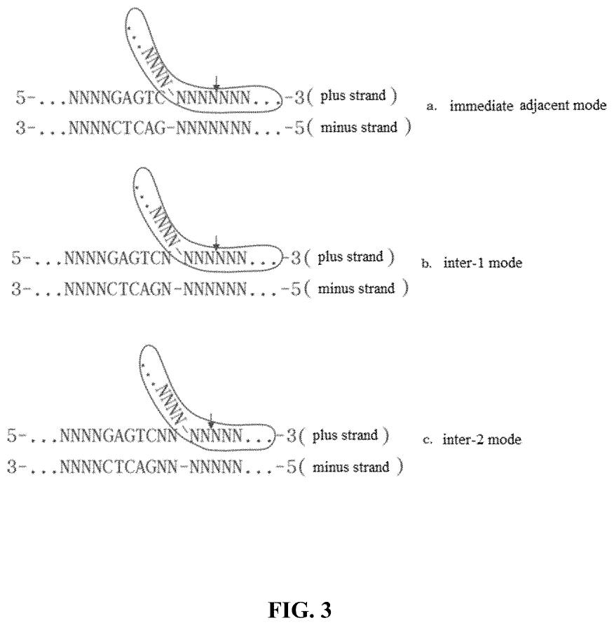

In some embodiments, said restriction nicking enzyme is Nt.BstNBI. This enzyme cleaves in the manner shown in FIG. 2 (the symbol “. . . ” in the figure indicates a DNA sequence of variable length, and this symbol is suitable for all figures of the present invention), and its cleavage position is between the fourth and fifth bases after the recognition sequence on the plus strand, and no cleavage occurs for sequences on the minus strand. Similar to FokI, when the base after GAGTC on the plus strand is replaced with a base on another DNA molecule, it can still undergo cleavage at the position specified by the arrow (FIG. 3). In FIG. 3, the target single-stranded DNA being cleaved has been circled in black, and the structure outside the black circle is called oligonucleotide adapter. A total of three types of hybridization are shown in FIG. 3, of which FIG. 3a shows the immediate adjacent mode, i.e., the cleaved target single-stranded DNA has no other bases between the hybridization start base and the GAGTC sequence, and the cleavage occurs between the fourth and fifth bases of the target single-stranded DNA involved in hybridization. FIG. 3b shows the inter-1 mode, where the hybridization start base is spaced by 1 base from the GAGTC sequence in the target single-stranded DNA and the cleavage occurs between the third and fourth bases of the target single-stranded DNA involved in the hybridization; FIG. 3c shows the inter-2 mode, where the hybridization start base is spaced by 2 bases from the GAGTC sequence in the target single-stranded DNA and the cleavage occurs between the second and third bases of the target single-stranded DNA involved in the hybridization. All 3 modes are very efficient in cleaving the target single strand at the specified position, and the target single strand does not need to contain the complete recognition sequence or a part of the recognition sequence. This method of enzymatic digestion of an arbitrary single strand with the participation of an oligonucleotide adapter is known as assisted single-stranded enzymatic digestion.

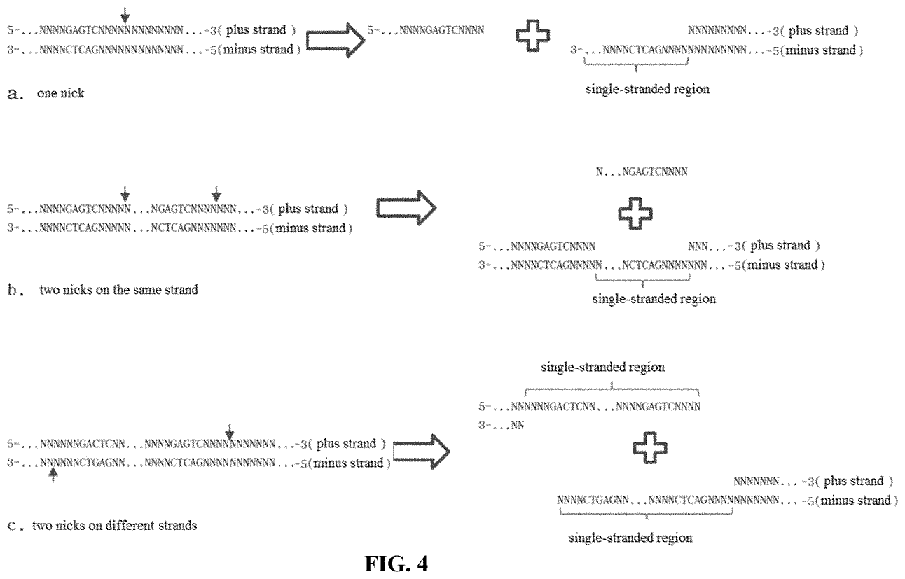

In some embodiments, said restriction nicking enzyme is Nt.BstNBI, and said generating a single-stranded region requires only one cleavage. Referring to FIG. 4a, a Nt.BstNBI recognition sequence is arranged near the 5′ end of the target double-stranded DNA, which, together with the sequence to its left and their complementary sequences, is additionally added to the 5′ end of the target double-stranded DNA, and by this addition, cleavage can be performed at any position without the limitations of the original sequence. After cleavage occurs, at a certain temperature, a small fragment of single strand, comprising the recognition sequence, detaches from the double-stranded DNA, generating a single-stranded region at the end of the double-stranded DNA (the position of the single-stranded region has been marked with a symbol in FIG. 4a), and this single-stranded region can be used as a substrate for the described assisted single-stranded enzyme digestion. This described way of generating a single-stranded region is called single-cleavage method.

In some embodiments, said restriction nicking enzyme is Nt.BstNBI, and the method of said generating a single-stranded region requires two cleavages, which can occur on the same strand (FIG. 4b) or on different strands (FIG. 4c). The two Nt.BstNBI recognition sequences that are identically oriented in FIG. 4b are both located on the sense strand, and these two Nt.BstNBI recognition sequences and the sequences between them, as well as their complementary sequences, are additionally added to the target double-stranded DNA. When both cleavage are completed, the single-stranded DNA between the two cleavages is separated from the double strand at a certain temperature, leaving a single-stranded region that can be used as a substrate for said assisted single-stranded enzyme digestion, this method is called same direction double cleavage. There are two Nt.BstNBI recognition sequences located on different strands in FIG. 4c, and these two Nt.BstNBI recognition sequences and the sequences between them, as well as their complementary sequences, are additionally added to the target double-stranded DNA. When both cleavages are completed, the double strand between the two cleavages undergoes denaturation and separation under a certain temperature, leaving a single-stranded region on the left and right side, and this single-stranded region can also be used as a substrate for the assisted single-stranded enzyme digestion described, this method is called opposite direction double-cleavage method.

In all embodiments, by a certain temperature, it is meant a temperature at which said single-stranded region can be generated while the activity of said restriction nicking enzyme activity can be ensured. This generally refers to 37 to 75 degrees Celsius, preferably 45 to 65 degrees Celsius, more preferably 53 to 63 degrees Celsius.

In all embodiments, said restriction nicking enzyme, in addition to Nt.BstNBI, can also be Nt.AlwI, simply by substituting the corresponding recognition sequence.

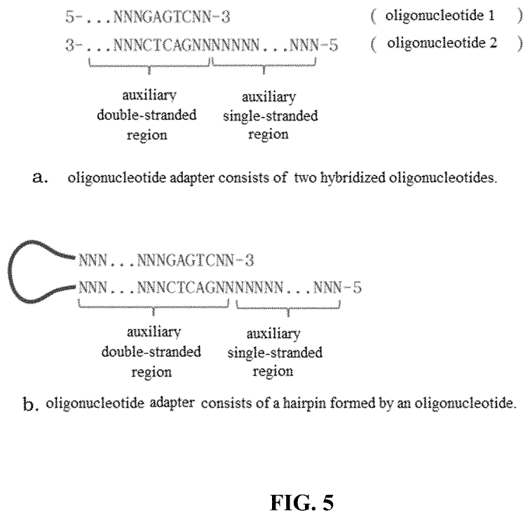

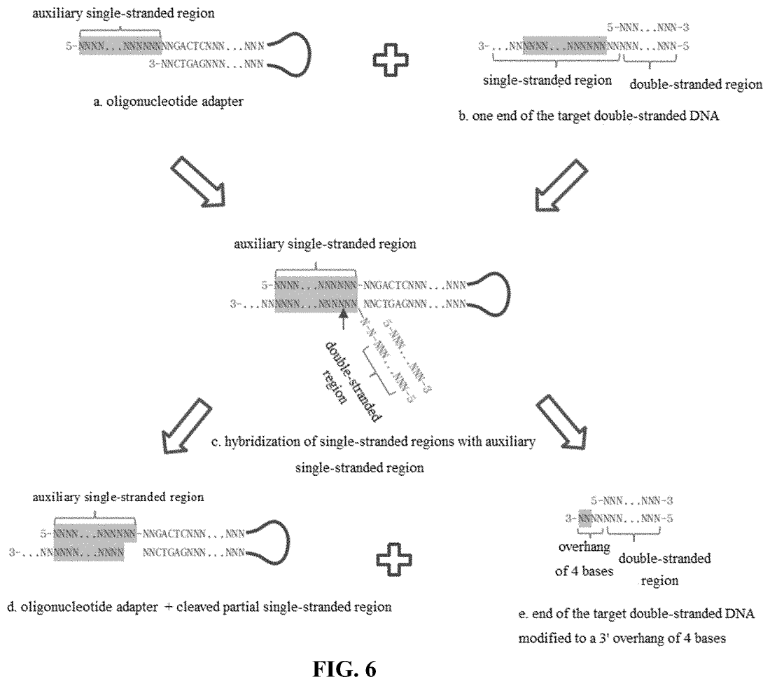

In some embodiments, said restriction nicking enzyme is Nt.BstNBI, said oligonucleotide adapter is formed by hybridization of two oligonucleotides (FIG. 5a), and in FIG. 5a, the 5′ end of oligonucleotide 2 is able to hybridize with said single-stranded region, referred to as the auxiliary single-stranded region (which may also be referred to as the single-stranded portion of the oligonucleotide adapter in the context of the present invention). FIG. 5a shows only the inter-2 mode described, other patterns can be obtained by adjusting the number of bases N at 3′ end of oligonucleotide 1. Said double-stranded region other than the auxiliary single-stranded region (referred to as the auxiliary double-stranded region, which in the present invention may also be referred to as the double-stranded portion of the oligonucleotide adapter) has a length of 10 to 50 bases, preferably 12 to 30 bases, and most preferably 15 to 20 bases.

In some embodiments, said restriction nicking enzyme is Nt.BstNBI, and said oligonucleotide adapter consists of an oligonucleotide forming a hairpin structure (FIG. 5b), in which the curved thick black line represents the closed loop portion of the hairpin, this way of drawing has the same meaning in all illustrations of the present invention. In FIG. 5b, the 5′ end single-stranded portion may be hybridized with said target single-stranded region, i.e., said auxiliary single-stranded region. FIG. 5b shows only the inter-2 mode described, other modes can be obtained by adjusting the number of bases N at 3′ end of the oligonucleotide. The hairpin structure described has a stem double-stranded region (referred to as the auxiliary double-stranded region) having a length of 6 to 30 bases, preferably 10 to 15 bases.

In all embodiments, said oligonucleotide adapter formed by hybridization of two oligonucleotides, and said oligonucleotide adapter consisting of one oligonucleotide forming a hairpin structure, are functionally interchangeable.

In all embodiments, hybridization between said single-stranded region and said auxiliary single-stranded region is achieved only if a certain percentage of said single-stranded region and said auxiliary single-stranded region in solution hybridize at said certain temperature. It is not necessary that said single-stranded region be of the same length as said auxiliary single-stranded region, nor is it necessary that the hybridization between said single-stranded region and said auxiliary single-stranded region strictly conform to the base-pairing principle.

In all embodiments, said single chain region, which may be generated by said single cleavage method, may be generated by said same direction double-cleavage method, or may be generated by said opposite direction double-cleavage method.

In some embodiments, said restriction nicking enzyme is Nt.BstNBI, when said single-stranded region is already present, a single strand is cleaved at the designated position on said single-stranded region under the combined action of said oligonucleotide adapter and said restriction nicking enzyme, generating a 3′ overhang of 4 bases at the end of said target double-stranded DNA (FIG. 6). The line between two bases in the illustration represents that these two bases are adjacent and covalently contiguous, only for the convenience of the drawing to make the two bases slightly distant from each other, while the two bases that are slightly distant without the line are covalently discontinuous from each other, and this way of drawing is applicable in all the illustrations of the present invention. The bases that undergo hybridization in the auxiliary single-stranded region and the single-stranded region in FIG. 6 have been highlighted by gray shading, the position of the cleavage of said assisted single-stranded enzyme digestion has been pointed out by arrows (FIG. 6c), and after completion of the cleavage, said oligonucleotide adapter remains hybridized with a portion of said single-stranded region generated by the cleavage (FIG. 6d), while the end of the target double-stranded DNA has been modified to a 3′ overhang of 4 bases (FIG. 6e). In combination with said generation of a single-stranded region and said assisted single-stranded enzyme digestion, the effect of double-stranded cleavage can be achieved at the end of double-stranded DNA, a process known as assisted double-stranded enzyme digestion.

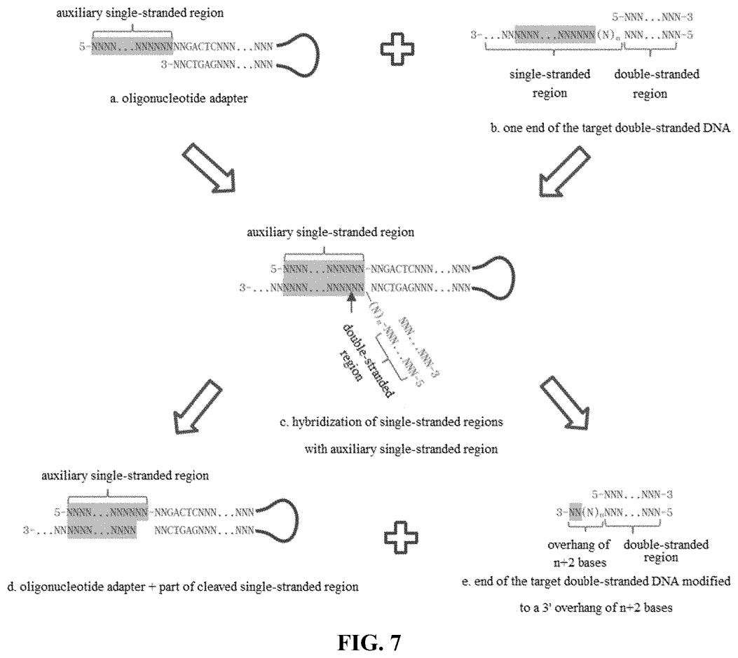

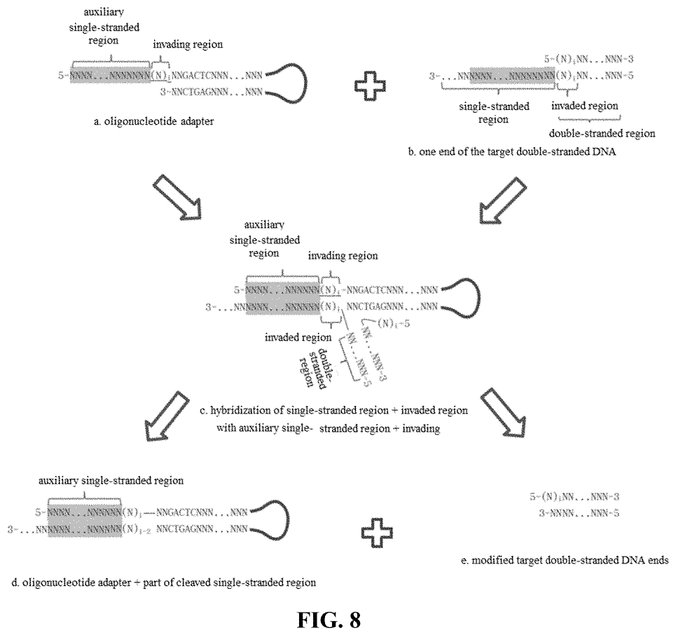

In some embodiments, said restriction nicking enzyme is Nt.BstNBI, when said single-stranded region is already present, a single strand is cleaved at the designated position on said single-stranded region under the combined action of said oligonucleotide adapter and said restriction nicking enzyme, generating a 3′ overhang of 3 bases or a 3′ overhang of 2 bases or any 3′ overhang of greater than 3 bases at the end of said target double-stranded DNA (FIG. 7). In FIG. 7b, the number of bases n between the hybridized region (gray shaded part) and the double-stranded region is variable in said single-stranded region. The corresponding assisted double-stranded enzyme digestion results in a 3′ overhang of n+2 bases. Said assisted double-stranded enzyme digestion includes the following.

1) when n=1, said assisted double-stranded enzyme digestion generates a 3′ overhang of 3 bases.

2) when n=0, said assisted double-stranded enzyme digestion generates a 3′ overhang of 2 bases.

3) when n=2, said assisted double-stranded enzyme digestion generates a 3′ overhang of 4 bases, i.e., the 3′ overhang of 4 bases shown in FIG. 6.

4) When n is greater than 2, a 3′ overhang of n+2 length is generated. It is noteworthy that the presence of a defined 5 bases (GACTC when said restriction nicking enzyme is Nt.BstNBI, see FIG. 4) in said single-stranded region of the present invention at a position 4 bases apart from the said double-stranded region is determined by said method of generating a single-stranded region. Thus, when n is greater than 2, the ends generated by said assisted double-stranded enzyme digestion will be subject to certain sequence restrictions. For example, when n=3, the hanging part generated at the end of the target double-stranded DNA is “NNNNG”, and the first four bases “N” can be customized, but the base “G” cannot be customized.

5) In said auxiliary single-stranded region, only 16 of said oligonucleotide adapters are required for cleavages in all n=2 caseswhen all but two bases at 3′ end are consistent. The mixture of these 16 oligonucleotide adapters is known as the universal adapter. In FIG. 7, said assisted double-stranded enzyme digestion results in two DNA molecules with the two structures shown in FIG. 7d and FIG. 7e, respectively. Only the structure in FIG. 7e is used for downstream operations, therefore, in the single-stranded region of the target double-stranded DNA, except for the base “N” of the hanging part, all other bases “N” in the single-stranded region can be fixed after being selected (which are additionally added to the target double-stranded DNA after being selected), and accordingly, all othe bases in the auxiliary single-stranded region except for the two bases at 3′ end can also be fixed.

In some embodiments, said restriction nicking enzyme is Nt.BstNBI, when said single-stranded region is already present, a single strand is cleaved at the designated position on said single-stranded region under the combined action of said oligonucleotide adapter and said restriction nicking enzyme, generating a 3′ overhang of 1 base, a blunt end, or a 5′ overhang of greater than 0 base at the end of said target double-stranded DNA (FIG. 8). Said oligonucleotide adapter has an additional single-stranded invading region between said auxiliary single-stranded region and said auxiliary double-stranded region, denoted in the figure by (N)ᵢ, where i represents the number of bases and is greater than or equal to 0. Said invading region is complementary to a segment of the double-stranded region at the end of the target DNA, and this segment of the target DNA double-stranded region sequence is labeled as the invaded region (FIG. 8b). When said auxiliary single-stranded region hybridizes with said single-stranded region, said invading region has the opportunity to invade said invaded region and form a substrate that can be recognized by said restriction nicking enzyme (FIG. 8c), at this point, the i bases at 5′ end of the target double-stranded DNA are in the single-stranded state. After the cleavage occurs, the state of the end of the target double strand is determined by the amount of i. When i=0, a 3′ overhang of 2 bases is generated, and no base is invaded, and the state is the same as the case of n=0 in FIG. 7; when i=1, a 3′ overhang of 1 base is generated; when i=2, a blunt end is generated; when i is greater than 2, a 5′ overhang end is generated, with a length of i−2. This type of enzyme digestion in which a double-strand is invaded is known as invasive assisted double-stranded enzyme digestion and is characterized by:

1) Unlike the case of generating 3′ overhangs of greater than 4 bases in length, all 5′ overhangs generated can be arbitrarily tailored, regardless of length;

2) Said invading region is between 1 to 100 bases in length, preferably between 1 to 30 bases, and more preferably between 3 to 20 bases; and

3) Since the invasion of double-strand needs to go over a certain energy barrier, its reaction will be slower than said assisted double-stranded enzyme digestion.

In all embodiments, said assisted double-stranded enzyme digestion and invasive assisted double-stranded enzyme digestion have the following characteristics.

1) After said target double-stranded DNA is double-stranded cleaved, most of the bases from said single-stranded region will still hybridize with said oligonucleotide adapter (FIG. 7d and FIG. 8d), which is a structure called “inactivator”.

2) After said target double-stranded DNA is double-stranded cleaved, the cleavage generates customizable ends (structures in FIG. 7e and FIG. 8e), which are called “customized ends”.

3) Said inactivator and said customized end cannot be rejoined back with ligase and are difficult to be spliced back by polymerase, so this enzyme digestion is asymmetric and irreversible, which allows said customized end to be involved in downstream operations without removing said “inactivator” in most cases, which is of obvious significance for many downstream operations.

The target double-stranded DNA treated with the method of the present invention is usually not cleaved again by restriction nicking enzymes in downstream operations, thus maintaining its sequence integrity. The probability of the recognition sequence for said restriction nicking enzyme occurring on a random double-stranded DNA sequence is related to the length of its recognition sequence. Nt.BstNBI, for example, has a recognition sequence of 5 bases, GAGTC. The probability of this sequence occurring on one of the strands of random double-stranded DNA is 1/1024, and on the other strand is also 1/1024. When cleaving double-stranded DNA with Nt.BstNBI to generate nicks, it is generally difficult to generate double-strand break directly because the average distance between these nicks is 512 bases, and the integrity of the double helix structure of double-stranded DNA is maintained as long as there are no two nicks present on each of the two strands and in close proximity. For the target double-stranded DNA treated with the method of the present invention, even if there are restriction nicking enzyme recognition sites in the middle of its sequence, these nicks will be rapidly repaired if ligases are present in the downstream operations, so the sequence integrity of the target double-stranded DNA treated with the method of the present invention is guaranteed in most cases during the subsequent operations.

If the ligase present in the downstream operation can only ligate ends longer than 12 hanging bases (e.g., Taq ligase), then all cleavage methods that can generate ends of 12 and fewer hanging bases will severely disrupt sequence integrity. The probability of this condition occurring is about 0.00002, or about once per 50,000 bases on average. This is comparable to the frequency of occurrence of restriction endonucleases with recognition sequences of 8 bases in length, 8-base restriction endonucleases with recognition sequences that are palindromes occur on average once per 65,000 bases, and 8-base restriction endonucleases with recognition sequences that are not palindromes occur on average once per 33,000 bases.

If a ligase presented in the downstream operation can rapidly ligate ends longer than 4 hanging bases (e.g., T4 DNA ligase), then all cleavage methodes that can generate ends of 4 or fewer hanging bases will severely disrupt sequence integrity. The probability of this condition occurring is about 0.0000086, or about once every 116,000 bases on average.

Based on the above calculations, it is evident that the frequency of such clevage methods that generate severe damage to sequence integrity is very low, and the vast majority of genes are between a few hundred bases and a few thousand bases in length. At this length, most of the sequence integrity of the sequences obtained after enzyme digestion with the present invention is maintained during downstream operations.

There are a variety of restriction nicking enzymes with different recognition sequences to choose from, for example, the recognition sequence of Nt.BspQI is GCTCTTC, and for T4 DNA ligase, disruption of sequence integrity occurs with a probability of, on average, only once in nearly 30 million bases. This number is much larger than the volume of the genomes of E. coli (about 4.7 million base pairs) and saccharomyces cerevisiae (about 12 million base pairs). It can be said that said assisted double-stranded enzyme digestion and said invasive assisted double-stranded enzyme digestion based on the Nt.BspQI restriction nicking enzyme are almost free of consideration whether the sequence integrity inside the double strand will be damaged, especially when it is used as a first restriction nicking enzyme for the second cleavage mode (i.e., the first restriction nicking enzyme is different from the second restriction nicking enzyme), and can be applied to DNA of megabase length manipulation (e.g., splicing and enzyme digestion), which is of particular interest in the current lack of tools for manipulation of very long DNA sequences.

Said assisted single-stranded enzyme digestion, said assisted double-stranded enzyme digestion, and said invasive assisted double-stranded enzyme digestion, are collectively referred to as assisted enzyme digestion.

Said assisted enzyme digestion has the following characteristics.

The optimal operating temperature is influenced by the activity profile for said restriction nicking enzymes, most of which are currently known to have activity profiles with a maximum point between 37 degrees Celsius and 70 degrees Celsius, especially 50 degrees Celsius.

The optimal operating temperature is also influenced by the pH in the reaction buffer, the type and concentration of metal cations and some additives.

The optimal operating temperature is also related to the hybridization stability of said auxiliary single-stranded region with said single-stranded region; too high temperature and decreased hybridization stability can leads to a decrease in the proportion of the structure used as a substrate, thus leading to a decrease in the speed of cleavage; too low temperature will cause said restriction nicking enzyme to remain bound to the cleaved substrate after cleavage is completed, leading to a decrease in the turnover number of said restriction nicking enzyme, which also reduces the speed of cleavage. This optimal operating temperature range is therefore somewhat narrower than the optimal operating temperature range for said restriction nicking enzymes themselves, and sometimes even a variation of only 3 degrees Celsius can lead to an exponential change in cleavage speed.

Said optimal operating temperature is also related to the production of said single-stranded region. Higher temperatures make it easier for said single-stranded region to be generated and thus more favorable for subsequent assisted single-stranded enzyme digestion.

In some embodiments, said assisted enzyme digestion, where the temperature of the cleavage process is fixed, is referred to as isothermal assisted enzyme digestion.

In some other embodiments, said assisted enzyme digestion, where the temperature of the cleavage process is variable, is referred to as non-isothermal assisted enzyme digestion.

By isothermal assisted enzyme digestion, it means that the enzyme digestion is carried out according to a temperature cycling method with full consideration of the optimal temperature for each step in the cleavage process. The generation of said single-stranded region generally requires a higher temperature, and the temperature of said assisted single-stranded enzyme digestion is generally slightly lower. Also said restriction nicking enzymes have optimal operating temperatures. Allowing the reaction temperature to cycle between higher and lower can effectively promote cleavage efficiency. The highest temperature of the cycle is between 50 to 75 degrees Celsius, preferably 55 to 65 degrees Celsius. The lowest temperature of the cycle is between 37 to 55 degrees Celsius, preferably 45 to 55 degrees Celsius. The duration of each cycle is between 30 seconds to 20 minutes, preferably 1 minute to 5 minutes.

In some embodiments, the addition of D-trehalose to said assisted enzyme digestion reaction system increases the rate of digestion, with a concentration of 0.1M to 2M, preferably 0.2M to 0.6M of D-trehalose.

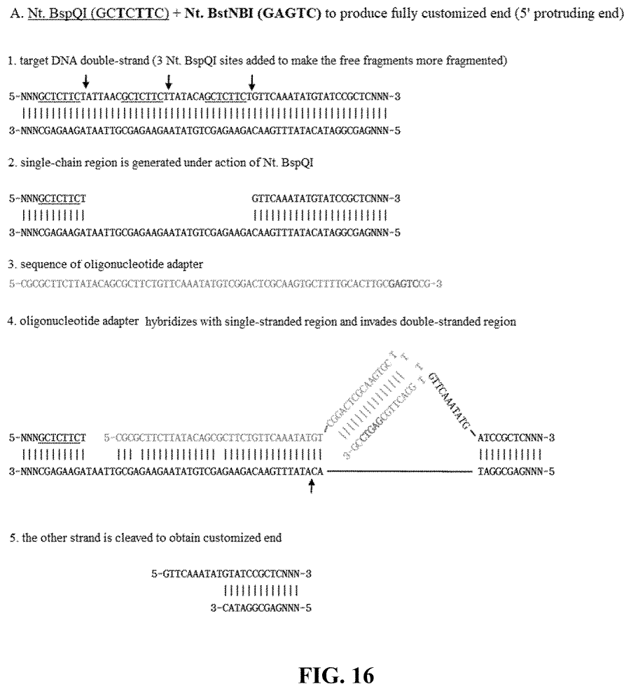

The restriction nicking enzyme used to generate a single-stranded region on the target double-stranded DNA (i.e., the first restriction nicking enzyme) and the restriction nicking enzyme used to cleave the other strand (i.e., the second restriction nicking enzyme) in the method of the present invention for manipulating the ends of double-stranded DNA may also be different. FIGS. 16-19 show examples of enzyme digestion patterns with Invasive assisted double-stranded enzyme digestion using two different restriction nicking enzymes, respectively.

In the enzyme digestion pattern shown in FIG. 16, the first restriction nicking enzyme is a restriction nicking enzyme of the Nt series, and the second restriction nicking enzyme is a restriction nicking enzyme of the Nt series.

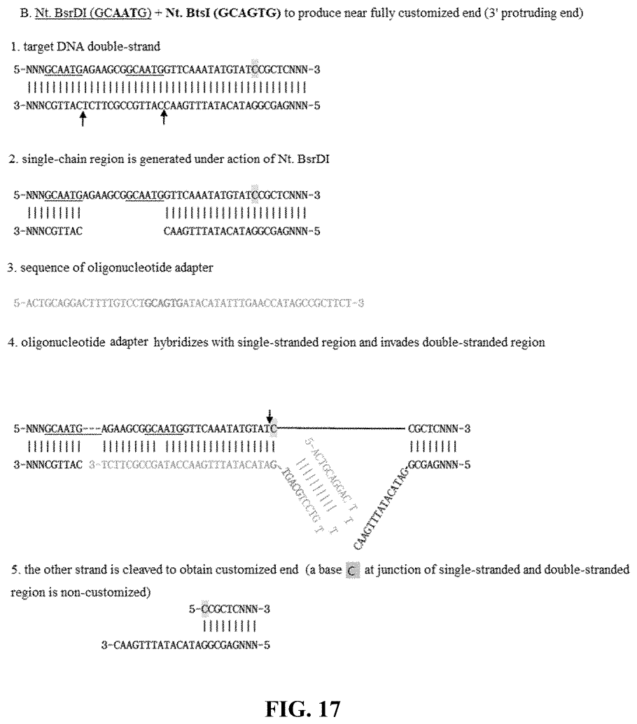

In the enzyme digestion pattern shown in FIG. 17, the first restriction nicking enzyme is a restriction nicking enzyme of the Nb series, and the second restriction nicking enzyme is a restriction nicking enzyme of the Nb series.

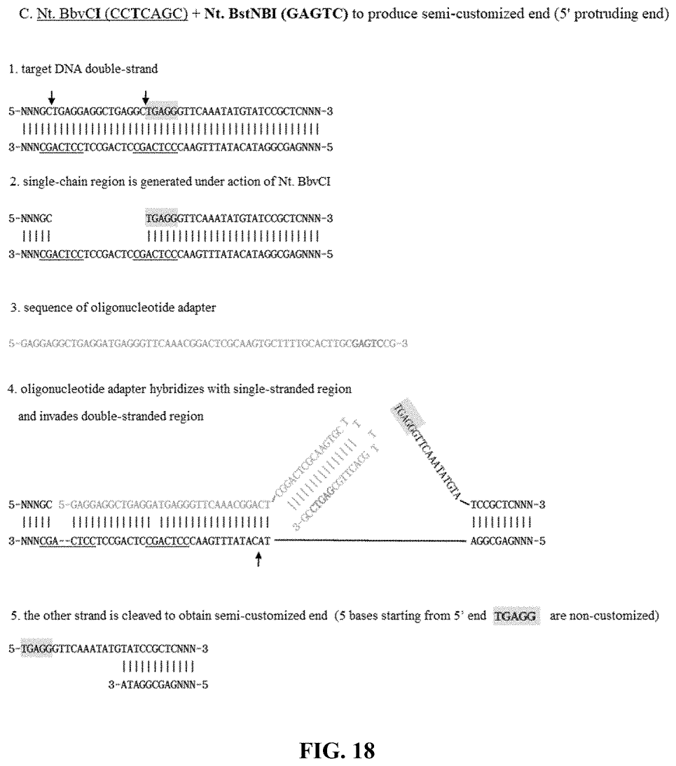

In the enzyme digestion pattern shown in FIG. 18, the first restriction nicking enzyme is a restriction nicking enzyme of the Nb series, and the second restriction nicking enzyme is a restriction nicking enzyme of the Nt series.

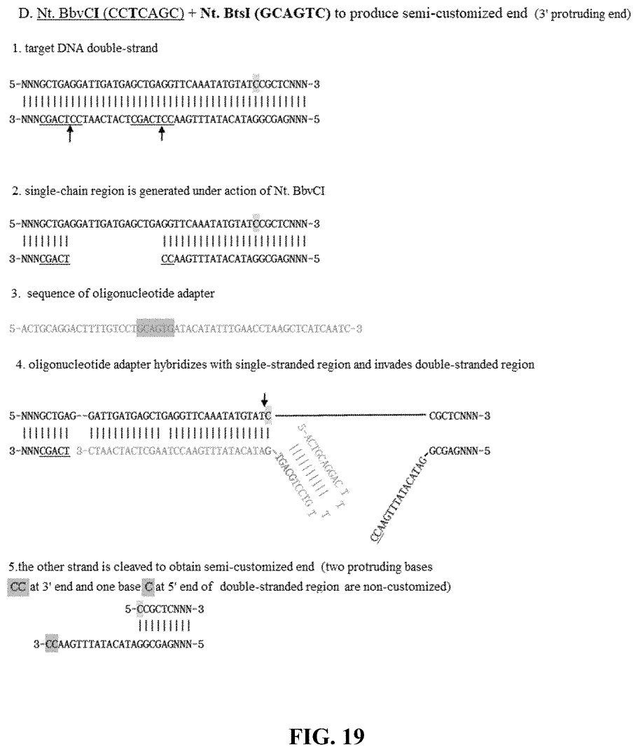

In the enzyme digestion pattern shown in FIG. 19, the first restriction nicking enzyme is a restriction nicking enzyme of the Nt series, and the second restriction nicking enzyme is a restriction nicking enzyme of the Nb series.

In these drawings, the arrow points to the cleavage position, the underline is the recognition sequence of the nicking enzyme that is used to generate the single-stranded region, the bold text is the recognition sequence of the nicking enzyme that cleaves the other strand, and the non-customized base is highlighted in the background (i.e., the base is defined and cannot be replaced).

The technical solutions of the present invention are described in further detail below by way of examples and in conjunction with the accompanying drawings, but the present invention is not limited to the following examples.

### EXAMPLE 1

### Assisted Single-Stranded Enzyme Digestion with Restriction Nicking Enzyme Nt.BstNBI

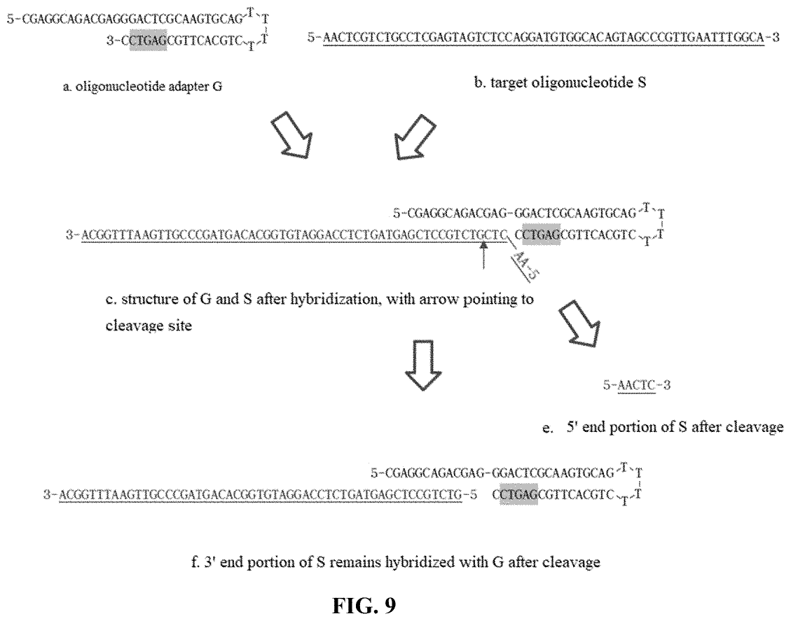

1. A target oligonucleotide S (FIG. 9b, the bases of S are underlined, including its cleaved bases) was synthesized with the sequence of

(SEQ ID NO: 1)
5′-AACTCGTCTGCCTCGAGTAGTCTCCAGGATGTGGCACAGTAGCCCGT
TGAATTTGGCA-3′.

2. An oligonucleotide adapter G based on the restriction nicking enzyme Nt.BstNBI (FIG. 9a, the recognition sequence of the restriction nicking enzyme has been highlighted by shading) was designed according to said inter-1 mode, consisting of an oligonucleotide forming a hairpin structure with the sequence of

(SEQ ID NO: 2)
5′-CGAGGCAGACGAGGGACTCGCAAGTGCAGTTTTCTGCACTTGCGAGT
CC-3′.

3. As designed, after hybridization of S and G, a cleavage occurs at the fifth and sixth bases of S (at the arrow in FIG. 9c) and a small 5-base fragment (FIGS. 9e) and 3′ end sequence of S that remained hybridized with G (FIG. 9f) are generated. The structure of FIG. 9f soon separated into two parts, G and S lacking 5 bases. G was also able to participate in the cleavage of next S. In contrast, the inhibition of the reaction will not be significant for S lacking 5 bases, as its hybridization with G is less stable than the hybridization of intact S with G.

4. According to the above design, the experimental steps were as follows.

(1) A reaction system V was established, with the components present in the system including:
\textcircled{1} 1× Cutsmart (product of NEB)
\textcircled{2} 0 0.6 mol/L of D-trehalose (Sangon)
\textcircled{3} 2 μmol/l oligonucleotide S
\textcircled{4} 0.2 μmol/l oligonucleotide adapter G
\textcircled{5} 2 U restriction nicking enzyme Nt.BstNBI
\textcircled{6} water balanced to 10 μl.

(2) The reaction system V was placed at 55 degrees Celsius for 1 hour.

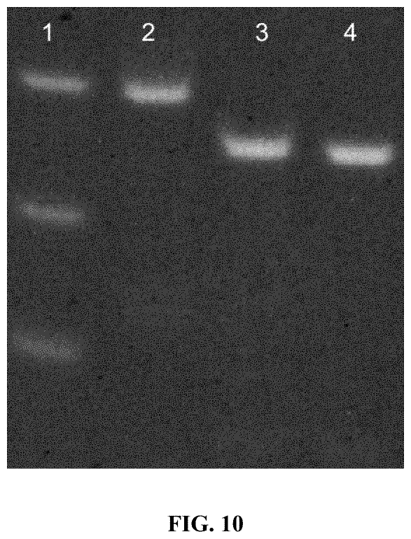

(3) 2.5 μl of V was taken and tested by 15% polyacrylamide gel electrophoresis with 4 mol/L of urea in the gel. The electrophoresis results were stained with EB and the electropherograms were shown in FIG. 10. Lane 1 in the figure was a single-stranded oligonucleotide marker and the lengths of the three bands from top to bottom were 58 bases, 48 bases and 41 bases, respectively. Lane 2 was the oligonucleotide S, which is 58 bases in length. Lane 3 was the reaction product V, which was formed by S being cleaved. If the cleavage position was as expected, two oligonucleotides of 53 bases and 5 bases were generated. The 5-base oligonucleotide was not detectable because the fragment was too small, but the 53-base fragment can be detected at a slightly lower position than the band in lane 2. Lane 4 was one oligonucleotide of 53 bases in length and served as a control. From the results, this cleavage position was in accordance with expectations.

5. The enzyme digestion speed was very fast. 2 units of Nt.BstNBI can completely cleave 20 pmol of substrate in less than 1 hour.

### EXAMPLE 2

### Assisted Double-Stranded Enzyme Digestion with Restriction Nicking Enzyme Nt.BstNBI to Generate a 3′ Overhang of 4 Bases

1. A double-stranded DNA sequence DS of 1104 bases in length was genetically synthesized with the following sequence.

(SEQ ID NO: 3)
TGTGTGGGTAGCAACCCCTGCTACAATCAGGGCACCTGTGAGCCCACATC
CGAGAACCCTTTCTACCGCTGTCTATGCCCTGCCAAATTCAACGGGCTAC
TGTGCCACATCCTGGACTACAGCTTCACAGGTGGCGCTGGGCGCGACATT
CCCCCACCGCAGATTGAGGAGGCCTGTGAGCTGCCTGAGTGCCAGGTGGA
TGCAGGCAATAAGGTCTGCAACCTGCAGTGTAATAATCACGCATGTGGCT
GGGATGGTGGCGACTGCTCCCTCAACTTCAATGACCCCTGGAAGAACTGC
ACGCAGTCTCTACAGTGCTGGAAGTATTTTAGCGACGGCCACTGTGACAG
CCAGTGCAACTCGGCCGGCTGCCTCTTTGATGGCTTCGACTGCCAGCTCA
CCGAGGGACAGTGCAACCCCCTGTATGACCAGTACTGCAAGGACCACTTC
AGTGATGGCCACTGCGACCAGGGCTGTAACAGTGCCGAATGTGAGTGGGA
TGGCCTAGACTGTGCTGAGCATGTACCCGAGCGGCTGGCAGCCGGCACCC
TGGTGCTGGTGGTGCTGCTTCCACCCGACCAGCTACGGAACAACTCCTTC
CACTTTCTGCGGGAGCTCAGCCACGTGCTGCACACCAACGTGGTCTTCAA
GCGTGATGCGCAAGGCCAGCAGATGATCTTCCCGTACTATGGCCACGAGG
AAGAGCTGCGCAAGCGAGTCTATACCACCGGAGTCGCATTACCCAATCAA
GCGCTCTACAGTGGGTTGGGCCACCTCTTCACTGCTTCCTGGTACCAGTG
GTGGGCGCCAGCGCAGGGAGCTGGACCCCATGGACATCCGTGGCTCCATT
GTCTACCTGGAGATCGACAACCGGCAATGTGTGCAGTCATCCTCGCAGTG
CTTCCAGAGTGCCACCGATGTGGCTGCCTTCCTAGGTGCTCTTGCGTCAC
TTGGCAGCCTCAATATTCCTTACAAGATTGAGGCCGTGAAGAGTGAGCCG
GTGGAGCCTCCGCTGCCCTCGCAGCTGCACCTCATGTACGTGGCAGCGGC
CGCCTTCGTGCTCCTGTTCTTTGTGGGCTGTGGGGTGCTGCTGTCCCGCA
AGCG.

2. This DS had a sequence DB of 24 bp in length at base 716 with the sequence GAGTCTATACCACCGGAGTCGCAT and two Nt.BstNBI recognition sequences in same direction on sequence DB were used to generate said single-stranded region.

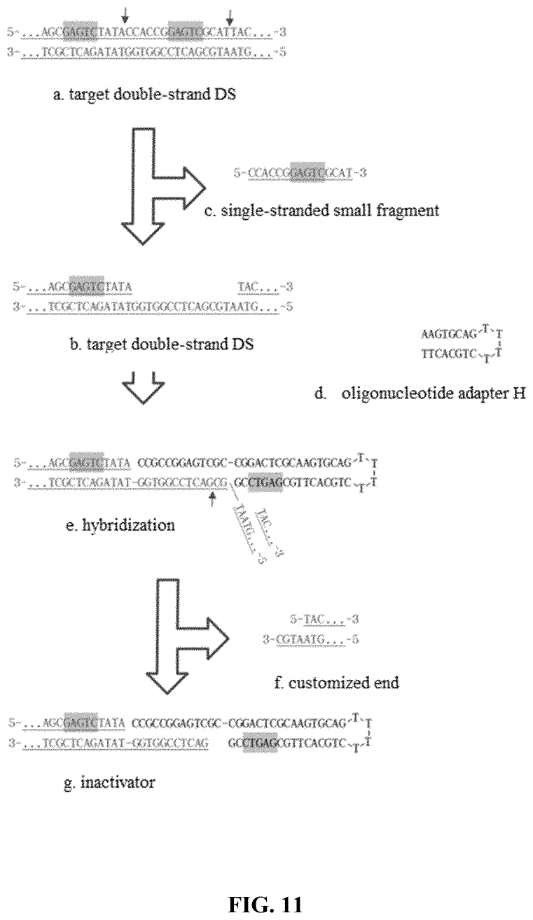

3. An oligonucleotide adapter H based on the restriction nicking enzyme Nt.BstNBI (FIG. 11d, the recognition sequence of the restriction nicking enzyme has been highlighted by shading) was designed according to said inter-2 model, consisting of an oligonucleotide forming a hairpin structure with the sequence of

(SEQ ID NO: 4)
5′-CCGCCGGAGTCGCCGGACTCGCAAGTGCAGTTTTCTGCACTTGCGAG
TCCG3′-

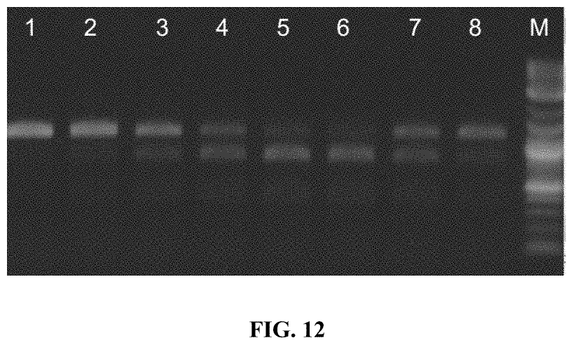

4. As designed, said assisted double-stranded enzyme digestion generated two double-stranded DNA products of 730 bp and 370 bp, and the enzyme digestion process was shown in FIG. 11. Figure lla shows the target double-strand DS, where only the bases associated with said assisted enzyme digestion are shown and the rest is replaced by “. . . ” instead. FIG. 11b shows a DS in which said single-stranded region had been generated, and a small fragment complementary to said single-stranded region (FIG. 11c) had departed from the DS. The oligonucleotide adapter H hybridized with the structure in FIG. 11b to generate the structure in FIG. 11e. The arrow in the figure indicated the position of cleavage. Upon completion of the cleavage, a 730 bp length of said inactivator (FIG. 11g) and a 370 bp length of said double-stranded DNA with customized end (FIG. 11f) were generated, wherein the customized end had a 3′ overhang of “ATGC”. FIG. 12 showed the electropherogram of the enzyme digestion, lane 1 showed the target DNA without digestion, lanes 2-8 showed the results of digestion at 48, 51, 54, 57, 60, 63 and 66 degrees Celsius for 1 hour, and lane 9 showed the 2-log DNA ladder from NEB.

### EXAMPLE 3

### Assisted Double-Stranded Enzyme Digestion with Restriction Nicking Enzyme Nt.BstNBI to Generate a 3′ Overhang of 4-Bases for Gene Splicing

1. The purpose of the example was to ligate a sequence to the pET28a (+) vector, starting with the modification of the polyclonal site of pET28a (+).

(1) The sequence near the polyclonal site of pET28a (+) was >MCS

(SEQ ID NO: 5)
. . . GTGGTGGTGGTGGTGGTGCTCGAGTGCGGCCGCAAGCTTGTCGA
CGGAGCTCGAATTCGGATCCGCGACCCATTTGCTGTCCACCAGTCATGCT
AGCCATATGGCTGCCGCGCGGCAC. . .,

where the underlined part is the sequence between XhoI-NdeI on the polyclonal site.

(2) The underlined part of the sequence was replaced by the following sequence, where the recognition sequence of Nt.BstNBI has been highlighted by a bolded font

(SEQ ID NO: 6)
CTCGACTCTGGTGGTTGACTCTTCAAATATGTATCCGCCTGCATGCAAGC
TTGAGTCATTGACCAGAGTCATG

(3) The modified pET28a (+) vector was referred to as pET28aMoD, and its sequence near the polyclonal site was >newMCS

(SEQ ID NO: 7)
. . . GTGGTGGTGGTGGTGGTGCTCGACTCTGGTGGTTGACTCTTCAA
ATATGTATCCGCCTGCATGCAAGCTTGAGTCATTGACCAGAGTCATGGCT
GCCGCGCGGCAC. . .

2. The sequence to be cloned into the pET28aMoD vector was the part of the following sequence without underline. The underlined part was for linkage to GCTC/ATGG at the sticky end of pET28aMoD, so GCTC was added at 5′ end and ATGG at 3′ end of the target sequence.

>Insert

(SEQ ID NO: 8)
GCTCGAGGGTCATTTGATTGATTATAATGGTAGAAATTATTCCTAATATC
AGGCAAGGGGATCAACACAGGTTCCAATTGTCTAACATCTGTTATTATGT
GGGGGAACATGGTGATCTGGCTGGGGCTCAAGCCTTCAGTTCGGAAATTT
GGTGGGACTGCTGCAAATATGATTTTCCCAGCGGTGAACGCGTTCCCCGC
GAGGATTACCTGCACTTCAAAACTACCTGCATAACCATTATACATTCTAG
ACAAGTGGGAAAGGTAGGGGTTCAAATCAGGGCCCAAGGACGCGCTCCAT
AGTATTTCACCTGGAGCGTTTCTAGGGGATACTGTAAACTCTCCACCAGG
GGCTTGTACAAAATTATTTCTAATCCAGGGGTCAATTACATTTTGTTGGC
CCGCTACAGGTGCCGCAATCGCGGCGCCAGCAACGGGTTCTAAAGCCATA
ACCTCATTGTTGACCTCTGGGACGAGGTTGGCCGAGGACCCATCAGATGG
GGTAGCGTCATTCGACGCCATCTTCATTCACAAAACTGGGAGCCAGATTG
CGATCGCCCTCCCACGTGCTCAGATCTGAGAATCTCATCCATCTGAACAT
TGGCTCTTGTCTGGGCACGTAAAAATCCATGCCACCTTCTTTTAGCTCTG
CAATGACTAATTTGCTGATTTTGCTGTAAAATGCTGGGCCATGAAGTGCG
GCCTCCCCCAGTAGGGACATCAATTGTATGGGTCTTTGGGAGTGTGGAAT
CATTGTTTCAGATGGATCTTCATGGTTGGAACCCCTAGTCCAGTACATTT
GCCTGAGCCATATGG.

3. Insert sequence was divided into 4 segments, and the followings were added at each end of each segment:

(1)

(SEQ ID NO: 9)
GCAAGCTTGAGTCATTGACCAGAGTC

was addedat 5′ end;

(2)

(SEQ ID NO: 10)
GACTCTGGTGGTTGACTCTTCAAA

was added at 3′ end. The sequences thus obtained were

>Insert_1
(SEQ ID NO: 11)
GCAAGCTTGAGTCATTGACCAGAGTCGCTCGAGGGTCATTTGATTGATTA
TAATGGTAGAAATTATTCCTAATATCAGGCAAGGGGATCAACACAGGTTC
CAATTGTCTAACATCTGTTATTATGTGGGGGAACATGGTGATCTGGCTGG
GGCTCAAGCCTTCAGTTCGGAAATTTGGTGGGACTGCTGCAAATATGATT
TTCCCAGCGGTGAACGCGTTCCCCGCGAGGATTACCTGCACTTCAAAACT
GACTCTGGTGGTTGACTCTTCAAA
>Insert_2
(SEQ ID NO: 12)
GCAAGCTTGAGTCATTGACCAGAGTCAACTACCTGCATAACCATTATACA
TTCTAGACAAGTGGGAAAGGTAGGGGTTCAAATCAGGGCCCAAGGACGCG
CTCCATAGTATTTCACCTGGAGCGTTTCTAGGGGATACTGTAAACTCTCC
ACCAGGGGCTTGTACAAAATTATTTCTAATCCAGGGGTCAATTACATTTT
GTTGGCCCGCTACAGGTGCCGCAATCGCGGCGCCAGCAACGGGGACTCTG
GTGGTTGACTCTTCAAA
>Insert_3
(SEQ ID NO: 13)
GCAAGCTTGAGTCATTGACCAGAGTCCGGGTTCTAAAGCCATAACCTCAT
TGTTGACCTCTGGGACGAGGTTGGCCGAGGACCCATCAGATGGGGTAGCG
TCATTCGACGCCATCTTCATTCACAAAACTGGGAGCCAGATTGCGATCGC
CCTCCCACGTGCTCAGATCTGAGAATCTCATCCATCTGAACATTGGCTCT
TGTCTGGGCACGTAAAAATCCATGCCACCTTCTTTTAGCTCTGCAATGAC
GACTCTGGTGGTTGACTCTTCAAA
>Insert_4
(SEQ ID NO: 14)
GCAAGCTTGAGTCATTGACCAGAGTCTGACTAATTTGCTGATTTTGCTGT
AAAATGCTGGGCCATGAAGTGCGGCCTCCCCCAGTAGGGACATCAATTGT
ATGGGTCTTTGGGAGTGTGGAATCATTGTTTCAGATGGATCTTCATGGTT
GGAACCCCTAGTCCAGTACATTTGCCTGAGCCATATGGGACTCTGGTGGT
TGACTCTTCAAA.

4. The four sequences of Insert_1, Insert_2, Insert_3, and Insert_4 were genetically, and the four DNAs were amplified by PCR using the same pair of primers PF/PR, and the PCR products were noted as P1, P2, P3, and P4, and the sequences of these PCR products were consistent with Insert_1, Insert_2, Insert_3, and Insert_4, respectively. The primer sequences were

(1)

(2)

| (SEQ ID NO: 15) | |
|---|---|
| PF | GCAAGCTTGAGTCATTGACC |
| (SEQ ID NO: 16) | |
| PR | TTTGAAGAGTCAACCACCAG. |

5. Sixteen oligonucleotide adapters were synthesized, and each oligonucleotide adapter had an oligonucleotide forming a hairpin structure, and these oligonucleotide adapters differed only at the tenth and eleventh bases, the sequences of which are shown in Table 1 below.

TABLE 1
16 oligonucleotides of the universal adapter

| No. | Sequence |
|---|---|
| 1 | ACCAGAGTCAACGGACTCGCAAGTGCAGTTTTCTGCACTTGCGAG |
| | TCCG |
| | (SEQ ID NO: 17) |
| 2 | ACCAGAGTCATCGGACTCGCAAGTGCAGTTTTCTGCACTTGCGAG |
| | TCCG |
| | (SEQ ID NO: 18) |
| 3 | ACCAGAGTCAGCGGACTCGCAAGTGCAGTTTTCTGCACTTGCGAG |
| | TCCG |
| | (SEQ ID NO: 19) |
| 4 | ACCAGAGTCACCGGACTCGCAAGTGCAGTTTTCTGCACTTGCGAG |
| | TCCG |
| | (SEQ ID NO: 20) |
| 5 | ACCAGAGTCTACGGACTCGCAAGTGCAGTTTTCTGCACTTGCGAG |
| | TCCG |
| | (SEQ ID NO: 21) |
| 6 | ACCAGAGTCTTCGGACTCGCAAGTGCAGTTTTCTGCACTTGCGAG |
| | TCCG |
| | (SEQ ID NO: 22) |
| 7 | ACCAGAGTCTGCGGACTCGCAAGTGCAGTTTTCTGCACTTGCGAG |
| | TCCG |
| | (SEQ ID NO: 23) |
| 8 | ACCAGAGTCTCCGGACTCGCAAGTGCAGTTTTCTGCACTTGCGAG |
| | TCCG |
| | (SEQ ID NO: 24) |
| 9 | ACCAGAGTCGACGGACTCGCAAGTGCAGTTTTCTGCACTTGCGAG |
| | TCCG |
| | (SEQ ID NO: 25) |
| 10 | ACCAGAGTCGTCGGACTCGCAAGTGCAGTTTTCTGCACTTGCGAG |
| | TCCG |
| | (SEQ ID NO: 26) |
| 11 | ACCAGAGTCGGCGGACTCGCAAGTGCAGTTTTCTGCACTTGCGAG |
| | TCCG |
| | (SEQ ID NO: 27) |
| 12 | ACCAGAGTCGCCGGACTCGCAAGTGCAGTTTTCTGCACTTGCGAG |
| | TCCG |
| | (SEQ ID NO: 28) |
| 13 | ACCAGAGTCCACGGACTCGCAAGTGCAGTTTTCTGCACTTGCGAG |
| | TCCG |
| | (SEQ ID NO: 29) |
| 14 | ACCAGAGTCCTCGGACTCGCAAGTGCAGTTTTCTGCACTTGCGAG |
| | TCCG |
| | (SEQ ID NO: 30) |
| 15 | ACCAGAGTCCGCGGACTCGCAAGTGCAGTTTTCTGCACTTGCGAG |
| | TCCG |
| | (SEQ ID NO: 31) |
| 16 | ACCAGAGTCCCCGGACTCGCAAGTGCAGTTTTCTGCACTTGCGAG |
| | TCCG |
| | (SEQ ID NO: 32) |

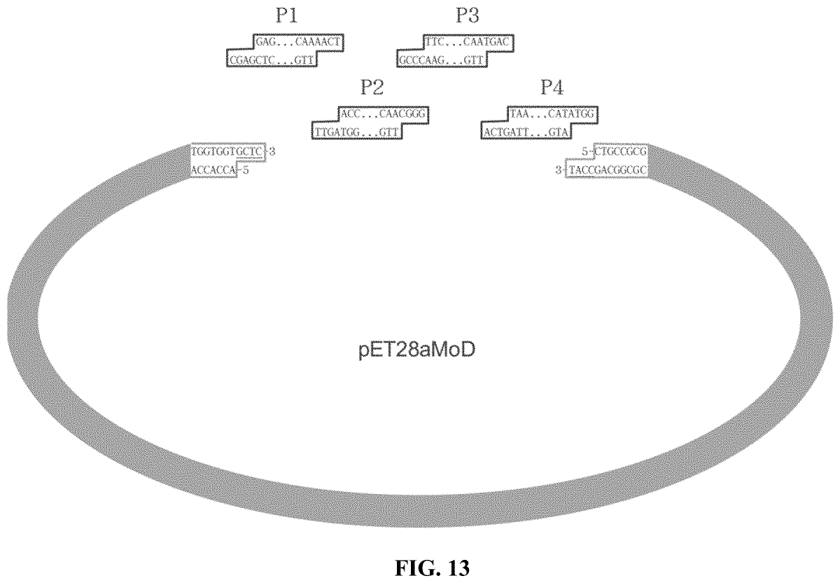

6. The oligonucleotides in Table 1 were mixed in equimolar fashion to obtain said universal adapter. This universal adapter could perform said assisted double-stranded enzyme digestion of P1/P2/P3/P4 as well as pET28aMoD and generated 3′ overhangs of 4 bases. Referring specifically to FIG. 13, only said customized end is shown, and not said inactivator. The two adjacent overhangs in the figure were complementary to each other, so that in the presence of a ligase, a circular plasmid that can be used for direct transformation could be generated.

7. According to the above method, the following reaction system X was established:

(1) 1× Cutsmart (NEB Corporation)

(2) 0.2 mol/L of D-trehalose (Sangon)

(3) 0.5 μmol/l total concentration of universal adapter

(4) 2 U of Nt.BstNBI (NEB)

(5) 17 ng of pET28aMoD plasmid

(6) 1.5 ng each of PCR products P1, P2, P3 and P4

(7) water balanced to a volume of 5 μl.

8. System X was subjected to said isothermal assisted enzyme digestion for 1 hour and the cycle was performed at 3 temperatures: 64 degrees Celsius for 1 second, 45 degrees Celsius for 1 minute, and 49 degrees Celsius for 1 minute, respectively.

9. When the cycle was over, an operation step at 70 degrees Celsius for 10 minutes can be performed to inactivate Nt.BstNBI or not. Because this enzyme was almost inactive at 10 degrees Celsius.

10. Additional 1 U of T4 DNA ligase (Thermo Fisher) was added to System X and supplemented with DTT to 5 mM, ATP to 0.5 mM, and PEG4000 to 5%. Ligation was carried out at 10 degrees Celsius for 1 hour.

11. Two microliters of System X was taken to directly transform DH5alpha E. coli and the colonies were cultured with kanamycin-resistant dishes.

12. About 100 colonies were observed the next day, and 16 colonies were picked for sequencing verification, 14 of which had sequences consistent with the Insert sequences, indicating that this method generated ends as expected and did not have other residual bases at the ends, and that the entire splicing process was seamless.

13. Most of the sequences can be cloned directly into the pET28a (+) vector using this method without the need to search for the right combination of restriction endonucleases from the numerous restriction endonucleases at the polyclonal sites. The modification method of this vector is also applicable to other vectors.

### EXAMPLE 4

### Invasive Assisted Double-Stranded Enzyme Digestion to Generate Blunt Ends and 5′ Hanging Ends

1. A target plasmid P was constructed as a substrate for enzymatic cleavage with the following sequence.

(SEQ ID NO: 33)
AAAAATAAACAAATAGGGGTTCCGCGCACATTTCCCCGAAAAGTGCCACC
TGACGTCTAAGAAACCATTATTATCATGACATTAACCTATAAAAATAGGC
GTATCACGAGGCCCTTTCGTTGTAAAACGACGGCCAGTCCGTCTCTATCC
GGTCTCGATCCGCAGTCTCTTGGCACAGGGAGTCTCGACCATAAGAGTCA
AGATGAATTGCAAGGTACTACTCAATTGTTGCCATCTGGTATCTGTGCTT
TCAGAGGTAGAGTTACTGCTCAAATCAACCAAAGAGACAGATGGCACATG
CAATTGCAAAACTTGAACGGTACTACTTACGACCCAACTGACGACGTTCC
AGCTCCATTGGGTACTCCAGACTTCAAGGGTGTTGTTTTCGGTATGGTTT
CTCAAAGAAACGTTGGTGACTCTGGATAAGCTGACTCACCGTGCGAGTTA
CTGCCAACCGAGACCCAACCGAGACGGGTCATAGCTGTTTCCAGTGTGCC
GCTTCCTCGCTCACTGACTCGCTGCGCTCGGTCGTTCGGCTGCGGCGAGC
GGTATCAGCTCACTCAAAGGCGGTAATACGGTTACCCACAGAATCAGGGG
ATAACGCAGGAAAGAACATGTGAGCAAAAGGCCAGCAAAAGGCCAGGAAC
CGTAAAAAGGCCGCGTTGCTGGCGTTTTTCCATAGGCTCCGCCCCCCTGA
CGAGCATCACAAAAATCGACGCTCAAGTCAGAGGTGGCGAAACCCGACAG
GACTATAAAGATACCAGGCGTTTCCCCCTGGAAGCTCCCTCGTGCGCTCT
CCTGTTCCGACCCTGCCGCTTACCGGATACCTGTCCGCCTTTCTCCCTTC
GGGAAGCGTGGCGCTTTCTCAATGCTCACGCTGTAGGTATCTCAGTTCGG
TGTAGGTCGTTCGCTCCAAGCTGGGCTGTGTGCACGAACCCCCCGTTCAG
CCCGACCGCTGCGCCTTATCCGGTAACTATCGTCTTGAGTCCAACCCGGT
AAGACACGACTTATCGCCACTGGCAGCAGCCACTGGTAACAGGATTAGCA
GAGCGAGGTATGTAGGCGGTGCTACAGAGTTCTTGAAGTGGTGGCCTAAC
TACGGCTACACTAGAAGGACAGTATTTGGTATCTGCGCTCTGCTGAAGCC
AGTTACCTTCGGAAAAAGAGTTGGTAGCTCTTGATCCGGCAAACAAACCA
CCGCTGGTAGCGGTGGTTTTTTTGTTTGCAAGCAGCAGATTACGCGCAGA
AAAAAAGGATCTCAAGAAGATCCTTTGATCTTTTCTACGGGGTCTGACGC
TCAGTGGAACGAAAACTCACGTTAAGGGATTTTGGTCATGAGATTATCAA
AAAGGATCTTCACCTAGATCCTTTTAAATTAAAAATGAAGTTTTAAATCA
ATCTAAAGTATATATGAGTAAACTTGGTCTGACAGTTACCAATGCTTAAT
CAGTGAGGCACCTATCTCAGCGATCTGTCTATTTCGTTCATCCATAGTTG
CCTGACTCCCCGTCGTGTAGATAACTACGATACGGGAGGGCTTACCATCT
GGCCCCAGTGCTGCAATAATACCGCGGGACCCACGCTCACCGGCTCCAGA
TTTATCAGCAATAAACCAGCCAGCCGGAAGGGCCGAGCGCAGAAGTGGTC
CTGCAACTTTATCCGCCTCCATCCAGTCTATTAATTGTTGCCGGGAAGCT
AGAGTAAGTAGTTCGCCAGTTAATAGTTTGCGCAACGTTGTTGCCATCGC
TACAGGCATCGTGGTGTCACGCTCGTCGTTTGGTATGGCTTCATTCAGCT
CCGGTTCCCAACGATCAAGGCGAGTTACATGATCCCCCATGTTGTGCAAA
AAAGCGGTTAGCTCCTTCGGTCCTCCGATCGTTGTCAGAAGTAAGTTGGC
CGCCGTGTTATCACTCATGGTTATGGCAGCACTACATAATTCTCTTACTG
TCATGCCATCCGTAAGATGCTTTTCTGTGACTGGTGAGTACTCAACCAAG
TCATTCTGAGAATAGTGTATGCGGCGACCGAGTTGCTCTTGCCCGGCGTC
AATACGGGATAATACCGCGCCACATAGCAGAACTTTAAAAGTGCTCATCA
TTGGAAAACGTTCTTCGGGGCGAAAACTCTCAAGGATCTTACCGCTGTTG
AGATCCAGTTCGATGTAACCCACTCGTGCACCCAACTGATCTTCAGCATC
TTTTACTTTCACCAGCGTTTCTGGGTGAGCAAAAACAGGAAGGCAAAATG
CCGCAAAAAAGGGAATAAGGGCGACACGGAAATGTTGAATACTCATACTC
TTCCTTTTTCAATATTATTGAAGCATTTATCAGGGTTATTGTCTCATGAG
CGGATACATATTTGAATGTATTTAG.

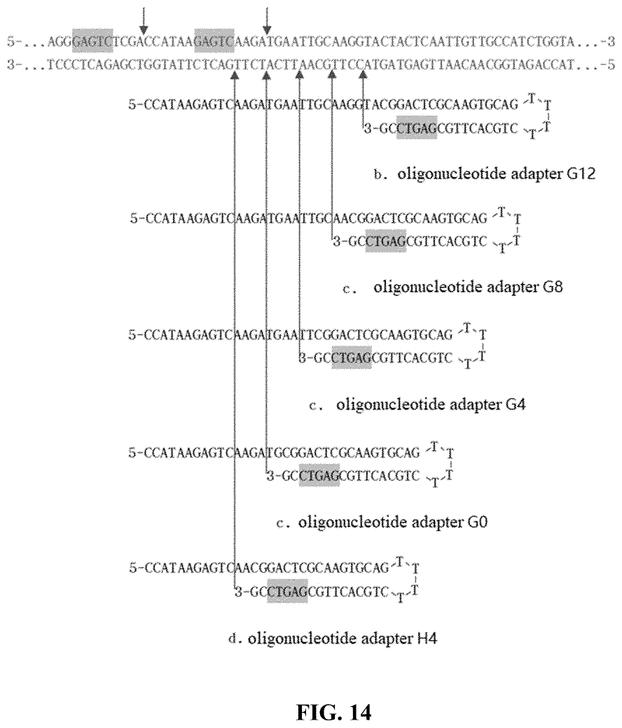

2. In this plasmid sequence, there are two said same direction double clevage sites that can be used for the generation of said single-stranded region, located on the plus and minus strands of the plasmid, respectively. FIG. 14 shows the sequence near said cleavage site on the plus strand. Those two arrows at the top of the schematic have pointed out the position of nicking of said same direction double-cleavage method, and after the cleavage, the sequence between the two arrows broke away from the double strand, leaving said single strand region on the plus strand. A total of 10 oligonucleotide adapters are shown in Table 2, and the cleavage positions of the first 5 oligonucleotide adapters on the minus strand have been indicated by the 5 upward arrows. The latter five are oligonucleotide adapters corresponding to the same direction double-cleavage sites on the minu strand, which act in the same way as in FIG. 14, so they are omitted here.

TABLE 2
Invasive oligonucleotide adapters

| Oligonucleotide | | |
|---|---|---|
| adapters | Sequences 5′-3′ | Types of ends |
| G12 | CCATAAGAGTCAAGATGAATTGCAAGGTACGGACTCGCAAGTGCAGTTT | 5′ overhang |
| | TCTGCACTTGCGAGTCCG (SEQ ID NO: 34) | |
| G8 | CCATAAGAGTCAAGATGAATTGCAACGGACTCGCAAGTGCAGTTTTCTG | 5′ overhang |
| | CACTTGCGAGTCCG (SEQ ID NO: 35) | of 8 bases |
| G4 | CCATAAGAGTCAAGATGAATTCGGACTCGCAAGTGCAGTTTTCTGCACT | 5′ overhang |
| | TGCGAGTCCG (SEQ ID NO: 36) | of 4 bases |
| G0 | CCATAAGAGTCAAGATGCGGACTCGCAAGTGCAGTTTTCTGCACTTGCG | blunt end |
| | AGTCCG (SEQ ID NO: 37) | |
| H4 | CCATAAGAGTCAACGGACTCGCAAGTGCAGTTTTCTGCACTTGCGAGTC | 3′ overhang |
| | CG (SEQ ID NO: 38) | of 4 bases |
| J12 | TATCCAGAGTCACCAACGTTTCTTTGAGACGGACTCGCAAGTCGAGTTT | 5′ overhang |
| | TCTGCACTTGCGAGTCCG (SEQ ID NO: 39) | of 12 bases |
| J8 | TATCCAGAGTCACCAACGTTTCTTTCGGACTCGCAAGTGCAGTTTTCTG | 5′ overhang |
| | CACTTGCGAGTCCG (SEQ ID NO: 40) | of 8 bases |
| J4 | TATCCAGAGTCACCAACGTTTCGGACTCGCAAGTGCAGTTTTCTGCACT | 5′ overhang |
| | TGCGAGTCCG (SEQ ID NO: 41) | of 4 bases |
| J0 | TATCCAGAGTCACCAACCGGACTCGCAAGTGCAGTTTTCTGCACTTGCG | blunt end |
| | AGTCCG (SEQ ID NO: 42) | |
| K4 | TATCCAGAGTCACCGGACTCGCAAGTCAGTTTTCTGCACTTGCGAGTCC | 3′ overhang |
| | G (SEQ ID NO: 43) | of 4 bases |

3. According to the above method, the following reaction system (Y) was established:

(1) 1× Cutsmart (NEB Corporation)

(2) 0.2 mol/L of D-trehalose (Sangon)

(3) 400 ng of plasmid P

(4) water balanced to a volume of 60 μl.

(4. The reaction system Y was divided into 12 aliquots of 5 μL each and recorded as

(1) P

(2) PN, supplemented by 2U Nt.BstNBI

(3) H4, supplemented with 2 U Nt.BstNBI, and then added oligonucleotide adapter H to a final concentration of 0.1 μmol/L

(4) GO, supplemented with 2 U Nt.BstNBI, and then added oligonucleotide adapter G0 to a final concentration of 0.1 μmol/L

(5) G4, supplemented with 2 U of Nt.BstNBI, and then added oligonucleotide adapter G4 to a final concentration of 0.1 μmol/L

(6) G8, supplemented with 2 U of Nt.BstNBI, and then added oligonucleotide adapter G8 to a final concentration of 0.1 μmol/L

(7) G12, supplemented with 2 U Nt.BstNBI, and then added oligonucleotide adapter G12 to a final concentration of 0.1 μmol/L

(8) K4, supplemented with 2 U of Nt.BstNBI, and then added oligonucleotide adapter K4 to a final concentration of 0.1 μmol/L

(9) J0, supplemented with 2 U of Nt.BstNBI, and then added oligonucleotide adapter J0 to a final concentration of 0.1 μmol/L

(10) J4, supplemented with 2 U Nt.BstNBI, and then added oligonucleotide adapter J4 to a final concentration of 0.1 μmol/L

(11) J8, supplemented with 2 U of Nt.BstNBI, and then added oligonucleotide adapter J8 to a final concentration of 0.1 μmol/L

(12) J12, supplemented with 2 U Nt.BstNBI, and then added oligonucleotide adapter J12 to a final concentration of 0.1 μmol/L.

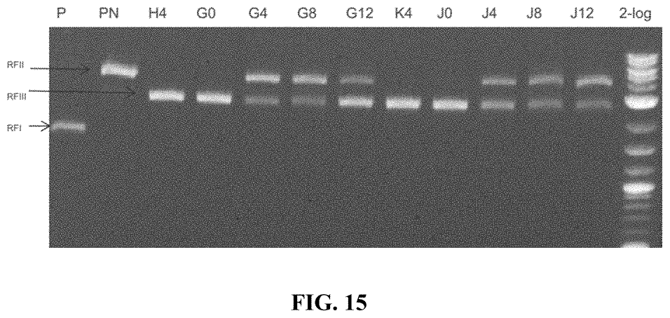

5. The above 12 samples were incubated at 55 degrees Celsius for 1 hour, then subjected to 1.5% agarose electrophoresis (FIG. 15).

6. From the electropherograms, all generated the corresponding linear plasmids, although the rates of the enzyme digestion differed.

7. The ends generated by these digestions were verified after sequencing.

### EXAMPLE 5

### Invasive Assisted Double-Stranded Enzyme Digestion Using Different Restriction Nicking Enzymes

1. In this example, the enzyme used to generate the single-stranded region (i.e., the first restriction nicking enzyme) was Nt.BspQI, while the enzyme used to cleave the other strand (i.e., the second restriction nicking enzyme) was Nt.BstNBI and the methylase was M .MlyI (the recognition sequence of this methylase is identical to that of Nt.BstNBI, so it achieves the same effect as M.BstBNI).

2. A plasmid was constructed for generating a single-stranded region via Nt.BspQI, with a core region sequence of (where the recognition sequence of Nt.B stQI has been highlighted by a bolded font):

(SEQ ID NO: 44)
GCTCTTCTATTAACGCTCTTCTTATACAGCTCTTCTGTTCAAATATGTAT
CCGCTCAATGAGACAATAACCCTGATAAATGCTTCAATAATATTGAAAAA
GGAAGAGTATGCAGTTTAAGGTTTACACCTATAAAAGAGAGAGCCGTTAT
CGTCTGTTTGTGGATGTACAGAGTGATATTATTGACACGCCAGGACGACG
GATGGTGATCCCCCTGGCCAGTGCACGTCTGCTGTCAGATAAAGTCCCCC
GTGAACTTTACCCGGTGGTGCATATCGGGGATGAAAGCTGGCGCATGATG
ACCACCGATATGGCCAGTGTGCCGGTCTCCGTTATCGGGGAAGAAGTGGC
TGATCTCAGCCACCGCGAAAATGACATCAAAAACGCCATTAACCTGATGT
TCTGGGGAATATAACAGAAGAGCATTTAAGGAAGAGCATAATTGGAAGAG
C

3. The M.MlyI gene sequence with the following sequence was added to the same plasmid for the methylation of this plasmid:

(SEQ ID NO: 45)
TTTTGTTTAACTTTAAGAAGGAGATATACCATGAAACCTATTTTAAAATA
TCGTGGTGGAAAAAAAGCAGAAATTCCTTTCTTTATTGACCATATACCCA
ATGATATCGAAACCTACTTTGAACCCTTTGTCGGGGGTGGTGCTGTATTC
TTCCATTTAGAACATGAAAAATCAGTTATCAATGATATTAATTCTAAGCT
TTATAAGTTCTATCTTCAATTAAAGCACAATTTTGATGAGGTAACTAAAC
AATTAAACGAACTACAGGAAATATATGAAAAAAACCAAAAGGAATATGAG
GAAAAAAAAGCTCTTGCTCCTGCTGGTGTCAGAGTGGAAAATAAAAATGA
AGAACTATATTATGAGCTAAGGAACGAATTTAACTATCCATCAGGAAAAT
GGCTAGACGCAGTAATTTATTATTTTATAAATAAAACTGCTTATAGTGGG
ATGATAAGGTATAACAGTAAAGGAGAATATAACGTTCCTTTTGGAAGATA
CAAAAACTTTAATACAAAAATCATTACTAAACAACACCATAACCTGCTTC
AAAAAACAGAAATATATAATAAAGATTTTTCTGAAATTTTTAAGATGGCA
AAACCAAATGACTTCATGTTTCTTGATCCTCCATATGATTGTATTTTTAG
TGATTATGGAAATATGGAGTTTACAGGTGATTTCGACGAGAGGGAACATC
GTAGGCTTGCTGAAGAGTTTAAAAACTTAAAGTGCCGTGCACTAATGATC
ATTAGTAAAACGGAATTAACTACCGAACTATATAAAGATTATATCGTTGA
TGAATATCATAAAAGCTATTCTGTAAACATTAGAAATAGATTTAAGAATG
AAGCAAAGCATTATATAATCAAGAACTATGATTATGTACGAAAAAATAAA
GAAGAAAAATATGAGCAACTTGAACTTATTCATTAG

4. The complete plasmid sequence was constructed as

(SEQ ID NO: 46)
TTTTGTTTAACTTTAAGAAGGAGATATACCATGAAACCTATTTTAAAATA
TCGTGGTGGAAAAAAAGCAGAAATTCCTTTCTTTATTGACCATATACCCA
ATGATATCGAAACCTACTTTGAACCCTTTGTCGGGGGTGGTGCTGTATTC
TTCCATTTAGAACATGAAAAATCAGTTATCAATGATATTAATTCTAAGCT
TTATAAGTTCTATCTTCAATTAAAGCACAATTTTGATGAGGTAACTAAAC
AATTAAACGAACTACAGGAAATATATGAAAAAAACCAAAAGGAATATGAG
GAAAAAAAAGCTCTTGCTCCTGCTGGTGTCAGAGTGGAAAATAAAAATGA
AGAACTATATTATGAGCTAAGGAACGAATTTAACTATCCATCAGGAAAAT
GGCTAGACGCAGTAATTTATTATTTTATAAATAAAACTGCTTATAGTGGG
ATGATAAGGTATAACAGTAAAGGAGAATATAACGTTCCTTTTGGAAGATA
CAAAAACTTTAATACAAAAATCATTACTAAACAACACCATAACCTGCTTC
AAAAAACAGAAATATATAATAAAGATTTTTCTGAAATTTTTAAGATGGCA
AAACCAAATGACTTCATGTTTCTTGATCCTCCATATGATTGTATTTTTAG
TGATTATGGAAATATGGAGTTTACAGGTGATTTCGACGAGAGGGAACATC
GTAGGCTTGCTGAAGAGTTTAAAAACTTAAAGTGCCGTGCACTAATGATC
ATTAGTAAAACGGAATTAACTACCGAACTATATAAAGATTATATCGTTGA
TGAATATCATAAAAGCTATTCTGTAAACATTAGAAATAGATTTAAGAATG
AAGCAAAGCATTATATAATCAAGAACTATGATTATGTACGAAAAAATAAA
GAAGAAAAATATGAGCAACTTGAACTTATTCATTAGGACAATACTGACCA
TTTAAATCATACCTGACCTCCATAGCAGAAAGTCAAAAGCCTCCGACCGG
AGGCTTTTGACTTGATCGGCACGTAAGAGGTTCCAACTTTCACCATAATG
AAATAAGATCACTACCGGGCGTATTTTTTGAGTTATCGAGATTTTCAGGA
GCTAAGGAAGCTAAAATGAGTATTCAACATTTCCGTGTCGCCCTTATTCC
CTTTTTTGCGGCATTTTGCCTTCCTGTTTTTGCTCACCCAGAAACGCTGG
TGAAAGTAAAAGATGCTGAAGATCAGTTGGGTGCACGAGTGGGTTACATC
GAACTGGATCTCAACAGCGGTAAGATCCTTGAGAGTTTACGCCCCGAAGA
ACGTTTTCCAATGATGAGCACTTTTAAAGTTCTGCTATGTGGCGCGGTAT
TATCCCGTATTGACGCCGGGCAAGAGCAACTCGGTCGCCGCATACACTAT
TCTCAGAATGACTTGGTTGAGTACTCACCAGTCACAGAAAAGCATCTCAC
GGATGGCATGACAGTAAGAGAATTATGCAGTGCTGCCATAACCATGAGTG
ATAACACTGCGGCCAACTTACTTCTGGCAACGATCGGAGGACCGAAGGAG
CTAACCGCTTTTTTGCACAACATGGGGGATCATGTAACTCGCCTTGATCG
TTGGGAACCGGAGCTGAATGAAGCCATACCAAACGACGAGCGTGACACCA
CGATGCCTGTAGCAATGGCAACAACGTTGCGCAAACTATTAACTGGCGAA
CTACTTACTCTAGCTTCCCGGCAACAATTAATAGACTGGATGGAGGCGGA
TAAAGTTGCAGGATCACTTCTGCGCTCGGCCCTCCCGGCTGGCTGGTTTA
TTGCTGATAAATCTGGAGCCGGTGAGCGTGGGTCTCGCGGTATCATTGCA
GCACTGGGGCCAGATGGTAAGCCCTCCCGCATCGTAGTTATCTACACGAC
GGGGAGTCAGGCAACTATGGATGAACGAAATAGACAGATCGCTGAGATAG
GTGCCTCACTGATTAAGCATTGGTAAGTGACCAAACAGGAAAAAACCGCC
CTTAACATGGCCCGCTTTATCAGAAGCCAGACATTAACGCTTCTGGAGAA
ACTCAACGAGCTGGACGCGGATGAACAGGCAGACATCTGTGAATCGCTTC
ACGACCACGCTGATGAGCTTTACCGCAGCTGCCTCGCGCGTTTCGGTGAT
GACGGTGAAAACCTCTGATGAGGGCCCAAATGTAATCACCTGGCTCACCT
TCGGGTGGGCCTTTCTGCGTTGCTGGCGTTTTTCCATAGGCTCCGCCCCC
CTGACGAGCATCACAAAAATCGATGCTCAAGTCAGAGGTGGCGAAACCCG
ACAGGACTATAAAGATACCAGGCGTTTCCCCCTGGAAGCTCCCTCGTGCG
CTCTCCTGTTCCGACCCTGCCGCTTACCGGATACCTGTCCGCCTTTCTCC
CTTCGGGAAGCGTGGCGCTTTCTCATAGCTCACGCTGTAGGTATCTCAGT
TCGGTGTAGGTCGTTCGCTCCAAGCTGGGCTGTGTGCACGAACCCCCCGT
TCAGCCCGACCGCTGCGCCTTATCCGGTAACTATCGTCTTGAGTCCAACC
CGGTAAGACACGACTTATCGCCACTGGCAGCAGCCACTGGTAACAGGATT
AGCAGAGCGAGGTATGTAGGCGGTGCTACAGAGTTCTTGAAGTGGTGGCC
TAACTACGGCTACACTAGAAGAACAGTATTTGGTATCTGCGCTCTGCTGA
AGCCAGTTACCTCGGAAAAAGAGTTGGTAGCTCTTGATCCGGCAAACAAA
CCACCGCTGGTAGCGGTGGTTTTTTTGTTTGCAAGCAGCAGATTACGCGC
AGAAAAAAAGGATCTCAAGAAGATCCTTTGATTTTCTACCGAAGAAAGGC
CCACCCGTGAAGGTGAGCCAGTGAGTTGATTGCAGTCCAGTTACGCTGGA
GTCGCTCTTCTATTAACGCTCTTCTTATACAGCTCTTCTGTTCAAATATG
TATCCGCTCAATGAGACAATAACCCTGATAAATGCTTCAATAATATTGAA
AAAGGAAGAGTATGCAGTTTAAGGTTTACACCTATAAAAGAGAGAGCCGT
TATCGTCTGTTTGTGGATGTACAGAGTGATATTATTGACACGCCAGGACG
ACGGATGGTGATCCCCCTGGCCAGTGCACGTCTGCTGTCAGATAAAGTCC
CCCGTGAACTTTACCCGGTGGTGCATATCGGGGATGAAAGCTGGCGCATG
ATGACCACCGATATGGCCAGTGTGCCGGTCTCCGTTATCGGGGAAGAAGT
GGCTGATCTCAGCCACCGCGAAAATGACATCAAAAACGCCATTAACCTGA
TGTTCTGGGGAATATAACAGAAGAGCATTTAAGGAAGAGCATAATTGGAA
GAGCCTTAAGACTTTATGCTTCCGGCTCGTATAATGTGTGG

5. an oligonucleotide adapter for assisted enzyme digestion was synthesized:
GEFZ69R CGCGCTTCTTATACAGCGCTTCTGTTCAAATATGTCGGACTCGCAAGTGCTTTTGCA CTTGCGAGTCCG (SEQ ID NO:47), this adapter was used to generate a 5′ protruding overhang of 10 bases.

6. a reaction system was formulated as follows:

(1) 1× cutsmart (NEB)

(2) 50 mM NaCl

(3) 30 ng target plasmid

(4) 2U Nt.BstNBI

(5) 2U Nt.BspQI

(6) 0.4 μM oligonucleotide adapter

7. The reaction temperature was 55 degrees for 1 hour.

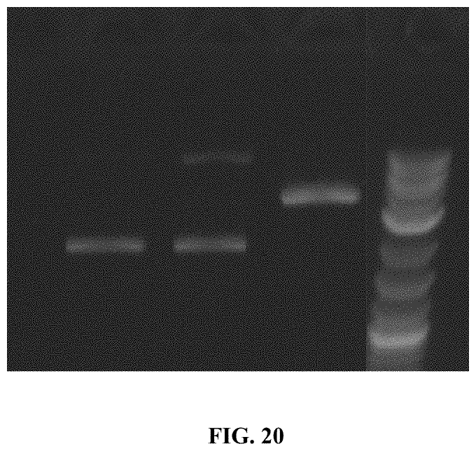

8. Electrophoretic detection showed that all plasmids were linearized (see FIG. 20). In FIG. 20, from left to right, lane 1 represented the above reaction system without Nt.BstNBI and Nt.BspQI, lane 2 represented the above reaction system without Nt.BspQI, lane 3 represented the above reaction system, and lane 4 represented DNA ladder 2-log from NEB, for plasmids with Nt.BstNBI added, only a very small amount of plasmids were digested, suggesting that the plasmids were not digested by Nt.BstNBI after methylation, and the trace digestion might be caused by the star activity of Nt.BstNBI.

9. Another linear plasmid was obtained by the above method, and the overhanging bases on the end of both sides were just complementary to the overhanging bases on both sides of this linear plasmid. After mixing the two linear plasmids, a large number of colonies could be obtained by direct transformation of E. coli without ligation; 10 colonies were picked and sequenced for verification and the result showed that the sequences were as expected, indicating that the end overhangs generated by this method were consistent with expectations.

The embodiments of the present invention are not limited to those described in the above examples, and without departing from the spirit and scope of the present invention, a person of ordinary skill in the art may make various changes and improvements in the form and details of the present invention, and these are considered to fall within the scope of protection of the present invention.

1.-25. (canceled)

26. A method of generating a cleavage at any predetermined position on a target single-stranded DNA, which comprises:
hybridizing a predetermined region of a target single-stranded DNA with a single-stranded portion of an oligonucleotide adapter, said oligonucleotide adapter is a DNA molecule having a double-stranded portion and a single-stranded portion, wherein the oligonucleotide adapter contains in the double-stranded portion a recognition site for the restriction nicking enzyme but lacks a sequence cleavable by the restriction nicking enzyme, wherein the single-stranded portion of the oligonucleotide adapter is capable of hybridizing with a predetermined region of the target single-stranded DNA to form a double-stranded structure that is recognizable by the restriction nicking enzyme, and thereby leaves the predetermined position on the target single-stranded DNA in a position that can be cleaved by said restriction nicking enzyme by recognition of the recognition site on the oligonucleotide adapter by said restriction nicking enzyme;
generating a cleavage at the predetermined position on said target single strand using said restriction nicking enzyme;
wherein the recognition sequence for said restriction nicking enzyme does not overlap with the cleavage position.

27. The method according to claim 26, wherein said restriction nicking enzyme is Nt.AlwI, Nt.BsmAI, Nt.BspQI, Nb.BsrDI, Nt.BstNBI or Nb.BtsI.

28. The method according to claim 26, wherein the oligonucleotide adapter is formed by hybridization of two oligonucleotides, the double-stranded portion is the portion of the two oligonucleotides that hybridize, the single-stranded portion is the portion of the two oligonucleotides that do not participate in the hybridization, and the restriction nicking enzyme recognition site is located in the double-stranded portion; alternatively the oligonucleotide adapter consists of an oligonucleotide that can form a hairpin structure, a stem of the hairpin includes a hybridized double-stranded portion and a single-stranded portion, and the restriction nicking enzyme recognition site is located in the double-stranded portion of the stem.

29. The method according to claim 26, wherein said restriction nicking enzyme recognition sequence in said oligonucleotide adapter is immediately adjacent to the end of the double-stranded portion on its cleavage site side, or wherein the restriction nicking enzyme recognition sequence in the oligonucleotide adapter is separated by one, two, or more nucleotides from the end of the double-stranded portion on its cleavage site side.

30. (canceled)

31. A method of generating a predetermined end of a double-stranded DNA, which comprises:
generating one or more nicks at a predetermined position on one strand of a target double-stranded DNA using a first restriction nicking enzyme to generate a single-stranded region on the other strand of the target double-stranded DNA;
using an oligonucleotide adapter having a recognition site for a second restriction nicking enzyme to hybridize with said single-stranded region in combination with use of the second restriction nicking enzyme to generate a cleavage at a predetermined position on the other strand of the target double-stranded DNA, eventually cleaving the target double-stranded DNA and generating the predetermined end at the cleavage site;
wherein said oligonucleotide adapter is a DNA molecule having a double-stranded portion and a single-stranded portion, said oligonucleotide adapter comprises the recognition site for said second restriction nicking enzyme and further comprises a complementary sequence to the cleavage site of said second restriction nicking enzyme but lacks a sequence that can be cleaved by said second restriction nicking enzyme, the single-stranded portion of said oligonucleotide adapter hybridizes with the single-stranded region of the target double-stranded DNA and forms, together with the double-stranded portion of said oligonucleotide adapter, a double-stranded structure recognizable and cleavable by said second restriction nicking enzyme, and thereby leaves the predetermined position on the other strand of the target double-stranded DNA in a position that can be cleaved by said restriction nicking enzyme by recognition of the recognition site on the oligonucleotide adapter by said second restriction nicking enzyme;
said first restriction nicking enzyme is the same as or different from said second restriction nicking enzyme;
wherein the first restriction nicking enzyme is a restriction nicking enzyme whose recognition sequence does not overlap with the cleavage position, or whose recognition sequence overlaps with the cleavage position; the second restriction nicking enzyme is a restriction nicking enzyme whose recognition sequence does not overlap with the cleavage position.

32. (canceled)

33. The method according to claim 31, wherein the second restriction nicking enzyme is Nt.AlwI, Nt.BsmAI, Nt.BspQI, Nb.BsrDI, Nt.BstNBI, or Nb.BtsI, and the first restriction nicking enzyme is Nt.AlwI, Nt.BsmAI, Nt.BspQI, Nb.BsrDI, Nt.BstNBI, Nb.BtsI, Nt.BbvCI, Nb.BbvCI, Nb.Bsm I or Nb.BssSI.

34. (canceled)

35. The method according to claim 31, wherein said generating a single-stranded region is to generate one nick or two nicks at the predetermined position on one strand of the target double-stranded DNA using the first restriction nicking enzyme such that a single-stranded region is generated after denaturing separation of the DNA double-strand between one nick and an end of the target double-stranded DNA or the double-stranded DNA between the two nicks.

36. The method according to claim 35, wherein said two nicks are located on the same strand of the target double-stranded DNA, or on different strands of the target double-stranded DNA.

37. The method according to claim 35, wherein said dissociation is carried out at 30 to 75 degrees Celsius.

38. The method according to claim 31, wherein the resulting single-stranded region has a length of 1 to 100 bases.

39. The method according to claim 31, wherein said oligonucleotide adapter comprises a double-stranded portion and a single-stranded portion, the oligonucleotide adapter comprises the second restriction nicking enzyme recognition sequence but lacks a sequence that can be cleaved by the second restriction nicking enzyme and has only its complementary sequence, the complementary sequence constitutes the single-stranded portion of said oligonucleotide adapter; the single-stranded portion of the oligonucleotide adapter can hybridize with the single-stranded region of the target double-stranded DNA, and the structure formed by hybridization of the oligonucleotide adapter and the target double-stranded DNA can be recognized by the second restriction nicking enzyme and cleaved at a predetermined position in or near the single-stranded region of the target double-stranded DNA.

40. The method according to claim 31, wherein said oligonucleotide adapter is formed by hybridization of two oligonucleotide chains, the double-stranded portion is the portion of the two oligonucleotides that hybridize, the single-stranded portion is the portion of the two oligonucleotides that do not participate in the hybridization; or said oligonucleotide adapter consists of an oligonucleotide chain that can form a hairpin structure, with the double-stranded portion being the stem portion of the hairpin and the single-stranded portion being the open-loop portion of the hairpin.

41. (canceled)

42. The method according to claim 31, wherein the region of the other strand of said target double-stranded DNA that hybridizes with the single-stranded portion of the oligonucleotide adapter is immediately adjacent to the double-stranded portion of said oligonucleotide adapter, and the region of the other strand of said target double-stranded DNA that hybridizes with the single-stranded portion of the oligonucleotide adapter is located on the single-stranded region of said target double-stranded DNA and is immediately adjacent to or one, two or more nucleotides apart from the double-stranded region of said target double-stranded DNA.

43. The method according to claim 42, wherein said second restriction nicking enzyme is Nt.BstNBI, Nt.AlwI, Nt.BspQI or Nt.BsmAI, said second restriction nicking enzyme recognition sequence in said oligonucleotide adapter is located in the double-stranded portion and said second restriction nicking enzyme recognition sequence is immediately adjacent to the end of the double-stranded portion on its cleavage site side, or the restriction nicking enzyme recognition sequence in the oligonucleotide adapter is separated by one, two, or more nucleotides from the end of the double-stranded portion on its cleavage site side.

44. The method according to claim 43, wherein the second restriction nicking enzyme used is Nt.AlwI or Nt.BstNBI, the number of nucleotides between the recognition sequence in said oligonucleotide adapter and 3′ end of the strand in which it is located is 2, and the number of nucleotides separating the hybridization region of the single-stranded region from the double-stranded region of the target double-stranded DNA is 2, eventually resulting in a 3′ overhang of 4 bases; the oligonucleotide adapter used is a mixture of 16 oligonucleotide adapters, the two single-stranded nucleotides immediately adjacent to the double-stranded portion of said 16 oligonucleotide adapters are different and all other nucleotides are identical, and said two single-stranded nucleotides immediately adjacent to the double-stranded portion include all permutations of the four kinds of nucleotides at these two positions.

45. The method according to claim 42, wherein said second restriction nicking enzyme is Nb.BsrDI or Nb.BtsI, the second restriction nicking enzyme recognition sequence in said oligonucleotide adapter is located on the strand having a single-stranded portion of the two strands of the double-stranded portion, one nucleotide at 3′ end of the recognition sequence is located on the single-stranded portion, other nucleotides of the recognition sequence are located on the double-stranded portion, and a partial sequence of the complementary sequence of the recognition sequence on the minus strand is located on the double-stranded portion and immediately adjacent to its 5′ end, said partial sequence is the nucleotides other than the last nucleotide at 5′ end of the complementary sequence on the minus strand.

46. The method according to claim 31, wherein the region of the other strand of said target double-stranded DNA that hybridizes with the single-stranded portion of the oligonucleotide adapter is immediately adjacent to the double-stranded portion of said oligonucleotide adapter; and the region on said target double-stranded DNA that hybridizes to the single-stranded portion of the oligonucleotide adapter includes not only the single-stranded region of that target double-stranded DNA, but also a portion of the double-stranded region sequence adjacent to that single-stranded region, said portion of the double-stranded region sequence adjacent to that single-stranded region is referred to as an invaded region; the single-stranded portion of said oligonucleotide adapter comprises a sequence capable of hybridizing with the single-stranded region of the target double-stranded DNA, and between that sequence and the double-stranded portion of the oligonucleotide adapter also includes a sequence capable of hybridizing with a segment of the double-stranded region of the target double-stranded DNA adjacent to the single-stranded region, which is referred to as an invading region.

47. The method according to claim 46, wherein the length of the invading region is between 1 to 100 bases.

48. The method according to claim 46, wherein said second restriction nicking enzyme is Nt.BstNBI, Nt.AlwI, Nt.BspQI or Nt.BsmAI, the second restriction nicking enzyme recognition sequence in said oligonucleotide adapter is located in the double-stranded portion and said second restriction nicking enzyme recognition sequence is immediately adjacent to the end of the double-stranded portion on its cleavage site side, or the restriction nicking enzyme recognition sequence in the oligonucleotide adapter is separated by one, two, or more nucleotides from the end of the double-stranded portion on its cleavage site side.

49. The method according to claim 46, wherein said second restriction nicking enzyme is Nb.BsrDI or Nb.BtsI, the second restriction nicking enzyme recognition sequence in said oligonucleotide adapter is located on the strand having a single-stranded portion of the two strands of the double-stranded portion, one nucleotide at 3′ end of the recognition sequence is located on the single-stranded portion, other nucleotides of the recognition sequence are located on the double-stranded portion, and a partial sequence of the complementary sequence on the minus strand of the recognition sequence is located on the double-stranded portion and immediately adjacent to its 5′ end, said partial sequence is the nucleotides other than the last nucleotide at 5′ end of the complementary sequence on the minus strand.

50. The method according to claim 31, wherein said method is carried out at a fixed temperature or a temperature cycle, said fixed temperature isbcing between 37 to 75 degrees Celsius, the maximum temperature of the cycle is between 50 to 75 degrees Celsius, the minimum temperature of the cycle is between 37 to 55 degrees Celsius, and the duration of each cycle is 30 seconds to 20 minutes.

51. (canceled)

52. The method according to claim 31, wherein said method is carried out in the presence of D-trehalose.

53. The method according to claim 31, wherein said first restriction nicking enzyme is different from said second restriction nicking enzyme and said target double-stranded DNA is methylated using a methylase of said second restriction nicking enzyme prior to generation of a single-stranded region of said double-stranded target DNA.

54. The method according to claim 53, wherein said methylation is carried out in vitro, or in vivo in a host cell.

55. The method according to claim 54, wherein said methylation is carried out in vivo in a host cell, wherein said target double-stranded DNA is located on the same DNA double-strand as the gene encoding said methylase, or wherein the gene encoding said methylase is located on a host cell chromosome such that the host cell expresses said methylase.

56. The method according to claim 53, wherein said second restriction nicking enzyme is Nt.BstNBI or Nt.AlwI and said methylase is M.BstNBI and M.AlwI, and said first restriction nicking enzyme is Nt.BspQI or Nb.BbvCI or Nt.BbvCI.

57. (canceled)


SEQUENCE LISTING (68 sequences)

SEQ ID NO: 1 - DNA, 58 bp, Artificial Sequence
  target oligonucleotide S
aactcgtctgcctcgagtagtctccaggatgtggcacagtagcccgttgaatttggca

SEQ ID NO: 2 - DNA, 49 bp, Artificial Sequence
  oligonucleotide adapter G
cgaggcagacgagggactcgcaagtgcagttttctgcacttgcgagtcc

SEQ ID NO: 3 - DNA, 1104 bp, Artificial Sequence
  double-stranded DNA sequence DS
  misc_feature (716)..(739): sequence DB comprising two Nt.BstNBI recognition sequences in same direction
tgtgtgggtagcaacccctgctacaatcagggcacctgtgagcccacatccgagaaccctttctaccgctgtctatgccctgccaaattcaacgggctactgtgccacatcctggactacagcttcacaggtggcgctgggcgcgacattcccccaccgcagattgaggaggcctgtgagctgcctgagtgccaggtggatgcaggcaataaggtctgcaacctgcagtgtaataatcacgcatgtggctgggatggtggcgactgctccctcaacttcaatgacccctggaagaactgcacgcagtctctacagtgctggaagtattttagcgacggccactgtgacagccagtgcaactcggccggctgcctctttgatggcttcgactgccagctcaccgagggacagtgcaaccccctgtatgaccagtactgcaaggaccacttcagtgatggccactgcgaccagggctgtaacagtgccgaatgtgagtgggatggcctagactgtgctgagcatgtacccgagcggctggcagccggcaccctggtgctggtggtgctgcttccacccgaccagctacggaacaactccttccactttctgcgggagctcagccacgtgctgcacaccaacgtggtcttcaagcgtgatgcgcaaggccagcagatgatcttcccgtactatggccacgaggaagagctgcgcaagcgagtctataccaccggagtcgcattacccaatcaagcgctctacagtgggttgggccacctcttcactgcttcctggtaccagtggtgggcgccagcgcagggagctggaccccatggacatccgtggctccattgtctacctggagatcgacaaccggcaatgtgtgcagtcatcctcgcagtgcttccagagtgccaccgatgtggctgccttcctaggtgctcttgcgtcacttggcagcctcaatattccttacaagattgaggccgtgaagagtgagccggtggagcctccgctgccctcgcagctgcacctcatgtacgtggcagcggccgccttcgtgctcctgttctttgtgggctgtggggtgctgctgtcccgcaagcg

SEQ ID NO: 4 - DNA, 51 bp, Artificial Sequence
  oligonucleotide adapter H
ccgccggagtcgccggactcgcaagtgcagttttctgcacttgcgagtccg

SEQ ID NO: 5 - DNA, 118 bp, Artificial Sequence
  sequence near the polyclonal site of pET28a (+)
  misc_feature (19)..(103): sequence between XhoI-NdeI on the polyclonal site
gtggtggtggtggtggtgctcgagtgcggccgcaagcttgtcgacggagctcgaattcggatccgcgacccatttgctgtccaccagtcatgctagccatatggctgccgcgcggcac

SEQ ID NO: 6 - DNA, 73 bp, Artificial Sequence
  sequence replacing the sequence between XhoI-NdeI of  pET28a (+)
  misc_feature (4)..(8): recognition sequence of Nt.BstNBI
  misc_feature (17)..(21): recognition sequence of Nt.BstNBI
  misc_feature (53)..(57): recognition sequence of Nt.BstNBI
  misc_feature (66)..(70): recognition sequence of Nt.BstNBI
ctcgactctggtggttgactcttcaaatatgtatccgcctgcatgcaagcttgagtcattgaccagagtcatg

SEQ ID NO: 7 - DNA, 106 bp, Artificial Sequence
  sequence near the polyclonal site of pET28aMoD
  misc_feature (19)..(91): sequence between XhoI-NdeI
  misc_feature (22)..(26): recognition sequence of Nt.BstNBI
  misc_feature (35)..(39): recognition sequence of Nt.BstNBI
  misc_feature (71)..(75): recognition sequence of Nt.BstNBI
  misc_feature (84)..(88): recognition sequence of Nt.BstNBI
gtggtggtggtggtggtgctcgactctggtggttgactcttcaaatatgtatccgcctgcatgcaagcttgagtcattgaccagagtcatggctgccgcgcggcac

SEQ ID NO: 8 - DNA, 815 bp, Artificial Sequence
  insert in pET28aMoD
  misc_feature (1)..(4): sequence for linkage to sticky end of pET28aMoD
  misc_feature (812)..(815): sequence for linkage to sticky end of pET28aMoD
gctcgagggtcatttgattgattataatggtagaaattattcctaatatcaggcaaggggatcaacacaggttccaattgtctaacatctgttattatgtgggggaacatggtgatctggctggggctcaagccttcagttcggaaatttggtgggactgctgcaaatatgattttcccagcggtgaacgcgttccccgcgaggattacctgcacttcaaaactacctgcataaccattatacattctagacaagtgggaaaggtaggggttcaaatcagggcccaaggacgcgctccatagtatttcacctggagcgtttctaggggatactgtaaactctccaccaggggcttgtacaaaattatttctaatccaggggtcaattacattttgttggcccgctacaggtgccgcaatcgcggcgccagcaacgggttctaaagccataacctcattgttgacctctgggacgaggttggccgaggacccatcagatggggtagcgtcattcgacgccatcttcattcacaaaactgggagccagattgcgatcgccctcccacgtgctcagatctgagaatctcatccatctgaacattggctcttgtctgggcacgtaaaaatccatgccaccttcttttagctctgcaatgactaatttgctgattttgctgtaaaatgctgggccatgaagtgcggcctcccccagtagggacatcaattgtatgggtctttgggagtgtggaatcattgtttcagatggatcttcatggttggaacccctagtccagtacatttgcctgagccatatgg

SEQ ID NO: 9 - DNA, 26 bp, Artificial Sequence
  sequence added to 5′ end of each segment of insert
gcaagcttgagtcattgaccagagtc

SEQ ID NO: 10 - DNA, 24 bp, Artificial Sequence
  sequence added to 3′ end of each segment of insert
gactctggtggttgactcttcaaa

SEQ ID NO: 11 - DNA, 274 bp, Artificial Sequence
  segment 1 of insert
gcaagcttgagtcattgaccagagtcgctcgagggtcatttgattgattataatggtagaaattattcctaatatcaggcaaggggatcaacacaggttccaattgtctaacatctgttattatgtgggggaacatggtgatctggctggggctcaagccttcagttcggaaatttggtgggactgctgcaaatatgattttcccagcggtgaacgcgttccccgcgaggattacctgcacttcaaaactgactctggtggttgactcttcaaa

SEQ ID NO: 12 - DNA, 267 bp, Artificial Sequence
  segment 2 of insert
gcaagcttgagtcattgaccagagtcaactacctgcataaccattatacattctagacaagtgggaaaggtaggggttcaaatcagggcccaaggacgcgctccatagtatttcacctggagcgtttctaggggatactgtaaactctccaccaggggcttgtacaaaattatttctaatccaggggtcaattacattttgttggcccgctacaggtgccgcaatcgcggcgccagcaacggggactctggtggttgactcttcaaa

SEQ ID NO: 13 - DNA, 274 bp, Artificial Sequence
  segment 3 of insert
gcaagcttgagtcattgaccagagtccgggttctaaagccataacctcattgttgacctctgggacgaggttggccgaggacccatcagatggggtagcgtcattcgacgccatcttcattcacaaaactgggagccagattgcgatcgccctcccacgtgctcagatctgagaatctcatccatctgaacattggctcttgtctgggcacgtaaaaatccatgccaccttcttttagctctgcaatgacgactctggtggttgactcttcaaa

SEQ ID NO: 14 - DNA, 212 bp, Artificial Sequence
  segment 4 of insert
gcaagcttgagtcattgaccagagtctgactaatttgctgattttgctgtaaaatgctgggccatgaagtgcggcctcccccagtagggacatcaattgtatgggtctttgggagtgtggaatcattgtttcagatggatcttcatggttggaacccctagtccagtacatttgcctgagccatatgggactctggtggttgactcttcaaa

SEQ ID NO: 15 - DNA, 20 bp, Artificial Sequence
  primer PF
gcaagcttgagtcattgacc

SEQ ID NO: 16 - DNA, 20 bp, Artificial Sequence
  primer PR
tttgaagagtcaaccaccag

SEQ ID NO: 17 - DNA, 49 bp, Artificial Sequence
  universal adapter
accagagtcaacggactcgcaagtgcagttttctgcacttgcgagtccg

SEQ ID NO: 18 - DNA, 49 bp, Artificial Sequence
  universal adapter
accagagtcatcggactcgcaagtgcagttttctgcacttgcgagtccg

SEQ ID NO: 19 - DNA, 49 bp, Artificial Sequence
  universal adapter
accagagtcagcggactcgcaagtgcagttttctgcacttgcgagtccg

SEQ ID NO: 20 - DNA, 49 bp, Artificial Sequence
  universal adapter
accagagtcaccggactcgcaagtgcagttttctgcacttgcgagtccg

SEQ ID NO: 21 - DNA, 49 bp, Artificial Sequence
  universal adapter
accagagtctacggactcgcaagtgcagttttctgcacttgcgagtccg

SEQ ID NO: 22 - DNA, 49 bp, Artificial Sequence
  universal adapter
accagagtcttcggactcgcaagtgcagttttctgcacttgcgagtccg

SEQ ID NO: 23 - DNA, 49 bp, Artificial Sequence
  universal adapter
accagagtctgcggactcgcaagtgcagttttctgcacttgcgagtccg

SEQ ID NO: 24 - DNA, 49 bp, Artificial Sequence
  universal adapter
accagagtctccggactcgcaagtgcagttttctgcacttgcgagtccg

SEQ ID NO: 25 - DNA, 49 bp, Artificial Sequence
  universal adapter
accagagtcgacggactcgcaagtgcagttttctgcacttgcgagtccg

SEQ ID NO: 26 - DNA, 49 bp, Artificial Sequence
  universal adapter
accagagtcgtcggactcgcaagtgcagttttctgcacttgcgagtccg

SEQ ID NO: 27 - DNA, 49 bp, Artificial Sequence
  universal adapter
accagagtcggcggactcgcaagtgcagttttctgcacttgcgagtccg

SEQ ID NO: 28 - DNA, 49 bp, Artificial Sequence
  universal adapter
accagagtcgccggactcgcaagtgcagttttctgcacttgcgagtccg

SEQ ID NO: 29 - DNA, 49 bp, Artificial Sequence
  universal adapter
accagagtccacggactcgcaagtgcagttttctgcacttgcgagtccg

SEQ ID NO: 30 - DNA, 49 bp, Artificial Sequence
  universal adapter
accagagtcctcggactcgcaagtgcagttttctgcacttgcgagtccg

SEQ ID NO: 31 - DNA, 49 bp, Artificial Sequence
  universal adapter
accagagtccgcggactcgcaagtgcagttttctgcacttgcgagtccg

SEQ ID NO: 32 - DNA, 49 bp, Artificial Sequence
  universal adapter
accagagtccccggactcgcaagtgcagttttctgcacttgcgagtccg

SEQ ID NO: 33 - DNA, 2375 bp, Artificial Sequence
  target plasmid P
aaaaataaacaaataggggttccgcgcacatttccccgaaaagtgccacctgacgtctaagaaaccattattatcatgacattaacctataaaaataggcgtatcacgaggccctttcgttgtaaaacgacggccagtccgtctctatccggtctcgatccgcagtctcttggcacagggagtctcgaccataagagtcaagatgaattgcaaggtactactcaattgttgccatctggtatctgtgctttcagaggtagagttactgctcaaatcaaccaaagagacagatggcacatgcaattgcaaaacttgaacggtactacttacgacccaactgacgacgttccagctccattgggtactccagacttcaagggtgttgttttcggtatggtttctcaaagaaacgttggtgactctggataagctgactcaccgtgcgagttactgccaaccgagacccaaccgagacgggtcatagctgtttccagtgtgccgcttcctcgctcactgactcgctgcgctcggtcgttcggctgcggcgagcggtatcagctcactcaaaggcggtaatacggttacccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaataataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgccgtgttatcactcatggttatggcagcactacataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttag

SEQ ID NO: 34 - DNA, 67 bp, Artificial Sequence
  oligonucleotide adapter G12
ccataagagtcaagatgaattgcaaggtacggactcgcaagtgcagttttctgcacttgcgagtccg

SEQ ID NO: 35 - DNA, 63 bp, Artificial Sequence
  oligonucleotide adapter G8
ccataagagtcaagatgaattgcaacggactcgcaagtgcagttttctgcacttgcgagtccg

SEQ ID NO: 36 - DNA, 59 bp, Artificial Sequence
  oligonucleotide adapter G4
ccataagagtcaagatgaattcggactcgcaagtgcagttttctgcacttgcgagtccg

SEQ ID NO: 37 - DNA, 55 bp, Artificial Sequence
  oligonucleotide adapter G0
ccataagagtcaagatgcggactcgcaagtgcagttttctgcacttgcgagtccg

SEQ ID NO: 38 - DNA, 51 bp, Artificial Sequence
  oligonucleotide adapter H4
ccataagagtcaacggactcgcaagtgcagttttctgcacttgcgagtccg

SEQ ID NO: 39 - DNA, 67 bp, Artificial Sequence
  oligonucleotide adapter J12
tatccagagtcaccaacgtttctttgagacggactcgcaagtgcagttttctgcacttgcgagtccg

SEQ ID NO: 40 - DNA, 63 bp, Artificial Sequence
  oligonucleotide adapter J8
tatccagagtcaccaacgtttctttcggactcgcaagtgcagttttctgcacttgcgagtccg

SEQ ID NO: 41 - DNA, 59 bp, Artificial Sequence
  oligonucleotide adapter J4
tatccagagtcaccaacgtttcggactcgcaagtgcagttttctgcacttgcgagtccg

SEQ ID NO: 42 - DNA, 55 bp, Artificial Sequence
  oligonucleotide adapter J0
tatccagagtcaccaaccggactcgcaagtgcagttttctgcacttgcgagtccg

SEQ ID NO: 43 - DNA, 51 bp, Artificial Sequence
  oligonucleotide adapter K4
tatccagagtcaccggactcgcaagtgcagttttctgcacttgcgagtccg

SEQ ID NO: 44 - DNA, 451 bp, Artificial Sequence
  core region sequence of the plasmid in Example 5
  misc_feature (1)..(7): recognition sequence of Nt.BstQI
  misc_feature (15)..(21): recognition sequence of Nt.BstQI
  misc_feature (29)..(35): recognition sequence of Nt.BstQI
  misc_feature (417)..(423): recognition sequence of Nt.BstQI
  misc_feature (431)..(437): recognition sequence of Nt.BstQI
  misc_feature (445)..(451): recognition sequence of Nt.BstQI
gctcttctattaacgctcttcttatacagctcttctgttcaaatatgtatccgctcaatgagacaataaccctgataaatgcttcaataatattgaaaaaggaagagtatgcagtttaaggtttacacctataaaagagagagccgttatcgtctgtttgtggatgtacagagtgatattattgacacgccaggacgacggatggtgatccccctggccagtgcacgtctgctgtcagataaagtcccccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataacagaagagcatttaaggaagagcataattggaagagc

SEQ ID NO: 45 - DNA, 936 bp, Artificial Sequence
  M.MlyI gene
ttttgtttaactttaagaaggagatataccatgaaacctattttaaaatatcgtggtggaaaaaaagcagaaattcctttctttattgaccatatacccaatgatatcgaaacctactttgaaccctttgtcgggggtggtgctgtattcttccatttagaacatgaaaaatcagttatcaatgatattaattctaagctttataagttctatcttcaattaaagcacaattttgatgaggtaactaaacaattaaacgaactacaggaaatatatgaaaaaaaccaaaaggaatatgaggaaaaaaaagctcttgctcctgctggtgtcagagtggaaaataaaaatgaagaactatattatgagctaaggaacgaatttaactatccatcaggaaaatggctagacgcagtaatttattattttataaataaaactgcttatagtgggatgataaggtataacagtaaaggagaatataacgttccttttggaagatacaaaaactttaatacaaaaatcattactaaacaacaccataacctgcttcaaaaaacagaaatatataataaagatttttctgaaatttttaagatggcaaaaccaaatgacttcatgtttcttgatcctccatatgattgtatttttagtgattatggaaatatggagtttacaggtgatttcgacgagagggaacatcgtaggcttgctgaagagtttaaaaacttaaagtgccgtgcactaatgatcattagtaaaacggaattaactaccgaactatataaagattatatcgttgatgaatatcataaaagctattctgtaaacattagaaatagatttaagaatgaagcaaagcattatataatcaagaactatgattatgtacgaaaaaataaagaagaaaaatatgagcaacttgaacttattcattag

SEQ ID NO: 46 - DNA, 3391 bp, Artificial Sequence
  complete plasmid sequence in Example 5
ttttgtttaactttaagaaggagatataccatgaaacctattttaaaatatcgtggtggaaaaaaagcagaaattcctttctttattgaccatatacccaatgatatcgaaacctactttgaaccctttgtcgggggtggtgctgtattcttccatttagaacatgaaaaatcagttatcaatgatattaattctaagctttataagttctatcttcaattaaagcacaattttgatgaggtaactaaacaattaaacgaactacaggaaatatatgaaaaaaaccaaaaggaatatgaggaaaaaaaagctcttgctcctgctggtgtcagagtggaaaataaaaatgaagaactatattatgagctaaggaacgaatttaactatccatcaggaaaatggctagacgcagtaatttattattttataaataaaactgcttatagtgggatgataaggtataacagtaaaggagaatataacgttccttttggaagatacaaaaactttaatacaaaaatcattactaaacaacaccataacctgcttcaaaaaacagaaatatataataaagatttttctgaaatttttaagatggcaaaaccaaatgacttcatgtttcttgatcctccatatgattgtatttttagtgattatggaaatatggagtttacaggtgatttcgacgagagggaacatcgtaggcttgctgaagagtttaaaaacttaaagtgccgtgcactaatgatcattagtaaaacggaattaactaccgaactatataaagattatatcgttgatgaatatcataaaagctattctgtaaacattagaaatagatttaagaatgaagcaaagcattatataatcaagaactatgattatgtacgaaaaaataaagaagaaaaatatgagcaacttgaacttattcattaggacaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagtttacgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatctcacggatggcatgacagtaagagaattatgcagtgctgccataaccatgagtgataacactgcggccaacttacttctggcaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggatcacttctgcgctcggccctcccggctggctggtttattgctgataaatctggagccggtgagcgtgggtctcgcggtatcattgcagcactggggccagatggtaagccctcccgcatcgtagttatctacacgacggggagtcaggcaactatggatgaacgaaatagacagatcgctgagataggtgcctcactgattaagcattggtaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgatgagggcccaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggcccacccgtgaaggtgagccagtgagttgattgcagtccagttacgctggagtcgctcttctattaacgctcttcttatacagctcttctgttcaaatatgtatccgctcaatgagacaataaccctgataaatgcttcaataatattgaaaaaggaagagtatgcagtttaaggtttacacctataaaagagagagccgttatcgtctgtttgtggatgtacagagtgatattattgacacgccaggacgacggatggtgatccccctggccagtgcacgtctgctgtcagataaagtcccccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataacagaagagcatttaaggaagagcataattggaagagccttaagactttatgcttccggctcgtataatgtgtgg

SEQ ID NO: 47 - DNA, 69 bp, Artificial Sequence
  oligonucleotide adapter for assisted enzyme digestion
cgcgcttcttatacagcgcttctgttcaaatatgtcggactcgcaagtgcttttgcacttgcgagtccg

SEQ ID NO: 48 - DNA, 24 bp, Artificial Sequence
  recognition sequence of FokI
  misc_feature (1)..(4): N is any nucleotide of A, T, C and G
  misc_feature (10)..(24): n is any nucleotide of A, T, C and G
nnnnggatgnnnnnnnnnnnnnnn

SEQ ID NO: 49 - DNA, 16 bp, Artificial Sequence
  recognition sequence of Nt.BstNBI
  misc_feature (1)..(4): n is a, c, g, or t
  misc_feature (10)..(16): n is a, c, g, or t
nnnngagtcnnnnnnn

SEQ ID NO: 50 - DNA, 11 bp, Artificial Sequence
  one end of nucleotide adapter
  misc_feature (1)..(4): n is a, c, g, or t
  misc_feature (10)..(11): one or both of these nucleotides can be absent
  misc_feature (10)..(11): n is a, c, g, or t
nnnngagtcnn

SEQ ID NO: 51 - DNA, 22 bp, Artificial Sequence
  sequence on target double-stranded  DNA
  misc_feature (1)..(4): n is a, c, g, or t
  misc_feature (10)..(22): n is a, c, g, or t
  misc_feature (14)..(22): these nucleotides can be absent
nnnngagtcnnnnnnnnnnnnn

SEQ ID NO: 52 - DNA, 14 bp, Artificial Sequence
  sequence on target double-stranded DNA
  misc_feature (1)..(4): n is a, c, g, or t
  misc_feature (10)..(14): n is a, c, g, or t
  misc_feature (14)..(14): this nucleotide can be absent
nnnngagtcnnnnn

SEQ ID NO: 53 - DNA, 13 bp, Artificial Sequence
  sequence on target double-stranded DNA
  misc_feature (1)..(1): n is a, c, g, or t
  misc_feature (7)..(13): n is a, c, g, or t
  misc_feature (11)..(13): these nucleotides can be absent
ngagtcnnnnnnn

SEQ ID NO: 54 - DNA, 13 bp, Artificial Sequence
  sequence on target double-stranded DNA
  misc_feature (1)..(6): n is a, c, g, or t
  misc_feature (12)..(13): n is a, c, g, or t
nnnnnngactcnn

SEQ ID NO: 55 - DNA, 20 bp, Artificial Sequence
  sequence on target double-stranded DNA
  misc_feature (1)..(4): n is a, c, g, or t
  misc_feature (10)..(20): n is a, c, g, or t
  misc_feature (14)..(20): these nucleotides can be absent
nnnngagtcnnnnnnnnnnn

SEQ ID NO: 56 - DNA, 10 bp, Artificial Sequence
  sequence on oligonucleotide adapter
  misc_feature (1)..(3): n is a, c, g, or t
  misc_feature (9)..(10): n is a, c, g, or t
nnngagtcnn

SEQ ID NO: 57 - DNA, 15 bp, Artificial Sequence
  sequence on oligonucleotide adapter
  misc_feature (1)..(7): n is a, c, g, or t
  misc_feature (13)..(15): n is a, c, g, or t
nnnnnnngactcnnn

SEQ ID NO: 58 - DNA, 16 bp, Artificial Sequence
  sequence on oligonucleotide adapter
  misc_feature (1)..(8): n is a, c, g, or t
  misc_feature (14)..(16): n is a, c, g, or t
nnnnnnnngactcnnn

SEQ ID NO: 59 - DNA, 19 bp, Artificial Sequence
  sequence on oligonucleotide adapter
  misc_feature (1)..(11): n is a, c, g, or t
  misc_feature (8)..(9): one or both of these nucleotides can be absent
  misc_feature (17)..(19): n is a, c, g, or t
nnnnnnnnnnngactcnnn

SEQ ID NO: 60 - DNA, 11 bp, Artificial Sequence
  end of pET28aMoD
tggtggtgctc

SEQ ID NO: 61 - DNA, 12 bp, Artificial Sequence
  end of pET28aMoD
cgcggcagccat

SEQ ID NO: 62 - DNA, 62 bp, Artificial Sequence
  target double-stranded DNA in Figure 16
  misc_feature (1)..(3): n is a, c, g, or t
  misc_feature (4)..(10): recognition sequence of Nt.BspQI
  misc_feature (12)..(39): these nucleotides can be cleaved by Nt.BspQI
  misc_feature (18)..(24): recognition sequence of Nt.BspQI
  misc_feature (32)..(38): recognition sequence of Nt.BspQI
  misc_feature (40)..(62): these nucleotides will be customized end
  misc_feature (60)..(62): n is a, c, g, or t
nnngctcttctattaacgctcttcttatacagctcttctgttcaaatatgtatccgctcnnn

SEQ ID NO: 63 - DNA, 45 bp, Artificial Sequence
  target double-stranded DNA in Figure 17
  misc_feature (1)..(3): n is a, c, g, or t
  misc_feature (4)..(9): recognition sequence of Nb.BsrDI
  misc_feature (10)..(22): the complementary sequence of these nucleotides on the minus strand can be cleaved by Nb.BsrDI
  misc_feature (17)..(22): recognition sequence of Nb.BsrDI
  misc_feature (23)..(45): the complementary sequence of these nucleotides on the minus strand will be the customized end
  misc_feature (43)..(45): n is a, c, g, or t
nnngcaatgagaagcggcaatggttcaaatatgtatccgctcnnn

SEQ ID NO: 64 - DNA, 52 bp, Artificial Sequence
  oligonucleotide adapter in Figure 17
actgcaggacttttgtcctgcagtgatacatatttgaaccatagccgcttct

SEQ ID NO: 65 - DNA, 48 bp, Artificial Sequence
  target double-stranded DNA in Figure 18
  misc_feature (1)..(3): n is a, c, g, or t
  misc_feature (3)..(10): the complementary sequence of these nucleotides on the minus strand is recognition sequence of Nb.BbvCI
  misc_feature (6)..(20): these nucleotides can be cleaved by Nb.BbvCI
  misc_feature (19)..(25): the complementary sequence of these nucleotides on the minus strand is recognition sequence of Nb.BbvCI
  misc_feature (21)..(48): these nucleotides will be customized end
  misc_feature (46)..(48): n is a, c, g, or t
nnngctgaggaggctgaggctgagggttcaaatatgtatccgctcnnn

SEQ ID NO: 66 - DNA, 60 bp, Artificial Sequence
  oligonucleotide adapter in Figure 18
gaggaggctgaggatgagggttcaaacggactcgcaagtgcttttgcacttgcgagtccg

SEQ ID NO: 67 - DNA, 47 bp, Artificial Sequence
  target double-stranded DNA in Figure 19
  misc_feature (1)..(3): n is a, c, g, or t
  misc_feature (3)..(10): the complementary sequence of these nucleotides on minus strand is recognition sequence of Nt.BbvCI
  misc_feature (9)..(23): the complementary sequence of these nucleotides on the minus strand can be cleaved by Nt.BbvCII
  misc_feature (19)..(25): the complementary sequence of these nucleotides on minus strand is recognition sequence of Nt.BbvCI
  misc_feature (24)..(47): the complementary sequence of these nucleotides on the minus strand will be customized end
  misc_feature (45)..(47): n is a, c, g, or t
nnngctgaggattgatgagctgaggttcaaatatgtatccgctcnnn

SEQ ID NO: 68 - DNA, 54 bp, Artificial Sequence
  oligonucleotide adapter in Figure 19
actgcaggacttttgtcctgcagtgatacatatttgaacctaagctcatcaatc